(19) United States
(12) Patent Application Publication
(10) Pub. No.: US 2021/0023175 A1
(43) Pub. Date: Jan. 28, 2021

(54) TP53 AS BIOMARKER FOR RESPONSIVENESS TO IMMUNOTHERAPY

(71) Applicant: Sanford Burnham Prebys Medical Discovery Institute, La Jolla, CA (US)

(72) Inventors: Robert WECHSLER-REYA, San Diego, CA (US); Alexandra GARANCHER, San Diego, CA (US); Carl WARE, Solana Beach, CA (US)

(21) Appl. No.: 16/642,001

(22) PCT Filed: Aug. 30, 2018
(86) PCT No.: PCT/US18/48916

§ 371 Date: Feb. 25, 2020

Related U.S. Application Data
(60) Provisional application No. 62/702,802, filed on Jul. 24, 2018.
(60) Provisional application No. 62/552,221, filed on Aug. 30, 2017.

(51) Int. Cl. A61K 38/19; A61P 35/00

(52) U.S. Cl. CPC A61K 38/191; A61K 45/06; A61P 35/00

(57) ABSTRACT

Disclosed herein are methods of treating a subject by an immunotherapy in combination with a low-dose of TNF-a or an LT receptor agonist as well as methods of identifying a cancer patient as having an increased or a reduced likelihood of responding to an immunotherapy by detection of TP53 gene status, in isolation, or in combination with assays for determining the levels of MHC-I and TP53 target genes. Also provided are methods of administering an immunotherapy to select, identified cancer patients.

### CROSS-REFERENCE

This application claims the benefit of U.S. Provisional Application No. 62/702,802 filed Jul. 24, 2018 and U.S. Provisional Application No. 62/552,221 filed Aug. 30, 2017, which areincorporated by reference herein in their entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if set forth in their entireties.

### BACKGROUND OF THE INVENTION

Somatic mutations in the TP53 gene are one of the most frequent alterations in human cancers.

### SUMMARY OF THE INVENTION

One embodiment provides a method of treating a patient having a cancer, comprising administering to the patient a low-dose of TNF-α or an LTβ receptor agonist, and an immunotherapy. In some embodiments, the patient has a loss-of-function TP53 mutation. In some embodiments, the immunotherapy comprises administering to the patient one or more immune checkpoint regulator, an adoptive T-cell therapy, a dendritic cell vaccination, or any combinations thereof. In some embodiments, the immune checkpoint regulator comprises an immune checkpoint inhibitor or an immune checkpoint activator. In some embodiments, the immune checkpoint activator is an agonist of costimulation by CD27, CD40, OX40, GITR, CD137, CD28, or ICOS. In some embodiments, the immune checkpoint activator is an agonist antibody that binds to CD27, CD40, OX40, GITR, CD137, CD28, or ICOS. In some embodiments, the immune checkpoint inhibitor is an antagonist of PD-1, PD-L1, CTLA-4, A2AR, B7-H3, B7-H4, BTLA, IDO, KIR, LAG3, TIM-3, VISTA, CD160, TIGIT or PSGL-1. In some embodiments, the immune checkpoint inhibitor is an antagonist antibody that binds to PD-1, PD-L1, CTLA-4, A2AR, B7-H3, B7-H4, BTLA, IDO, KIR, LAG3, TIM-3, VISTA, CD160, TIGIT or PSGL-1. In some embodiments, the cancer comprises a solid tumor, lymphoma or leukemia. In some embodiments, the cancer is medulloblastoma. In some embodiments, the method comprises administering a low dose of TNF-α and an anti-PD-1 antibody. In some embodiments, the method comprises administering an LTβ receptor agonist and an anti-PD-1 antibody. In some embodiments, the low dose of TNF-α or the low dose of the LTβ receptor agonist, and the immunotherapy, are administered concurrently. In some embodiments, the low dose of TNF-α or the low dose of the LTβ receptor agonist, and the immunotherapy, are administered sequentially. In some embodiments, the low dose of TNF-α and the anti-PD-1 antibody are administered concurrently. In some embodiments, the low dose of TNF-α and the anti-PD-1 antibody are administered sequentially. In some embodiments, the low dose of TNF-α comprises a dose that is about 100 fold to about 300 fold lower than a maximum tolerated dose of TNF-α in human. In some embodiments, the maximum tolerated dose of TNF-α in human comprises about 200 μg/m2 to about 400 μg/m2. In some embodiments, the low dose of TNF-α comprises a dose of at least about 0.6 μg/m2 to about 40 μg/m2. In some embodiments, the patient has previously been identified as having a reduced likelihood of responding to the immunotherapy. In some embodiments, the patient has previously been identified as having a reduced likelihood of response to the immunotherapy by a method comprising the steps of: obtaining a biological sample from said patient and detecting whether the biological sample comprises a loss-of-function TP53 mutation; and identifying said patient as having a reduced likelihood of response to the immunotherapy if the biological sample comprises the loss-of-function TP53 mutation. In some embodiments, the biological sample comprises a tumor sample. In some embodiments, the patient has previously been identified as having a reduced likelihood of response to the immunotherapy by a method comprising the steps of: obtaining a tumor sample from said patient and assaying levels of ERAP1 and TAP1 in said tumor sample; and identifying said patient as having a reduced likelihood of response to the immunotherapy if the levels of ERAP1 or TAP1, or both, are lower in the tumor sample than in a reference non-tumor biological sample. In some embodiments, the method further comprises assaying a level of MHC-I in the tumor sample and identifying said patient as having a reduced likelihood of response to the immunotherapy if the level of MHC-I is lower in the tumor sample than in the reference non-tumor biological sample. In some embodiments, the patient has previously been identified as having a reduced likelihood of response to the immunotherapy by a method comprising the steps of: obtaining a tumor sample from said patient and assaying a level of MHC-I in said tumor sample; and identifying said patient as having a reduced likelihood of response to the immunotherapy if the MHC-I level is lower in the tumor sample than in a reference non-tumor biological sample. In some embodiments, the method further comprises assaying levels of ERAP1 and TAP1 in the tumor sample and identifying said patient as having a reduced likelihood of response to the immunotherapy if the levels of ERAP1 and TAP1, or both are lower in the tumor sample than in the reference non-tumor biological sample. In some embodiments, the patient has previously been identified as having a reduced likelihood of response to the immunotherapy by a method comprising the steps of: obtaining a tumor sample from said patient and performing the following steps: detecting whether the tumor sample comprises a loss-of-function TP53 mutation, and assaying a level of at least one of MHC-I, ERAP1, and TAP1 in said tumor sample; and identifying said patient as having a reduced likelihood of response to the immunotherapy if the tumor sample comprises a loss-of-function TP53 mutation or if the level of at least one of MHC class 1, ERAP1, and TAP1 in the tumor sample is lower than that in a reference non-tumor biological sample. In some embodiments, the method comprises detecting whether the tumor sample comprises the loss-of-function TP53 mutation prior to assaying the level of at least one of MHC-I, ERAP1, and TAP1 in the tumor sample. In some embodiments, the method comprises assaying the level of at least one of MHC-I, ERAP1, and TAP1 in the tumor sample prior to detecting whether the tumor sample comprises the loss-of-function TP53 mutation. In some embodiments, the reference non-tumor biological sample is isolated from the same patient.

One embodiment provides a method of identifying a cancer patient as having an increased likelihood of response to an immunotherapy, said method comprising the steps of:
(i) obtaining a biological sample from said patient and detecting whether the biological sample comprises a loss-of-function TP53 mutation; and
(ii) identifying said patient as having an increased likelihood of response to the immunotherapy if the biological sample does not comprise the loss-of-function TP53 mutation and identifying said patient as having a reduced likelihood of response to the immunotherapy if the biological sample comprises the loss-of-function TP53 mutation.

In some embodiments, the immunotherapy is not administered to the patient identified as having the reduced likelihood of response in step (ii), thereby avoiding immunotherapy related side effects in said patient. In some embodiments, the method further comprises administering the immunotherapy to the patient identified as having the increased likelihood of response in step (ii). In some embodiments, the method further comprises administering a therapy comprising TNF-alpha to the patient identified as having the reduced likelihood of response in step (ii). In some embodiments, the immunotherapy involves T-cell based recognition of MHC-I. In some embodiments, immunotherapy comprises administration of one or more immune checkpoint regulators, adoptive T-cell therapy, dendritic cell vaccination, or any combinations thereof. In some embodiments, the immune checkpoint regulator comprises an immune checkpoint inhibitor or an immune checkpoint activator. In some embodiments, the immune checkpoint activator is an agonist of costimulation by CD27, CD40, OX40, GITR, CD137, CD28, or ICOS. In some embodiments, the immune checkpoint activator is an agonist antibody that binds to CD27, CD40, OX40, GITR, CD137, CD28, or ICOS. In some embodiments, the immune checkpoint inhibitor is an antagonist of PD-1, PD-L1, CTLA-4, A2AR, B7-H3, B7-H4, BTLA, IDO, KIR, LAG3, TIM-3, VISTA, CD160, TIGIT or PSGL-1. In some embodiments, the immune checkpoint inhibitor is an antagonist antibody that binds to PD-1, PD-L1, CTLA-4, A2AR, B7-H3, B7-H4, BTLA, IDO, KIR, LAG3, TIM-3, VISTA, CD160, TIGIT or PSGL-1. In some embodiments, the cancer comprises a solid tumor, lymphoma, or leukemia. In some embodiments, the cancer is medulloblastoma. In some embodiments, the detection is carried out by DNA sequencing of TP53 gene isolated from the biological sample, by measuring the expression of TP53 protein in the biological sample, or by RNA expression analysis of TP53 target genes. In some embodiments, the TP53 target genes comprise ERAP1 and TAP1. In some embodiments, identifying a patient as having the reduced likelihood of response to the immunotherapy reduces the risk of side effects associated with administering the immunotherapy to the patient without any therapeutic benefit.

One embodiment provides a method for treating a patient having a cancer, the method comprising administering an immunotherapy to the patient if and only if the patient does not comprise a loss-of-function TP53 mutation. Another embodiment provides a method for treating a patient having a cancer comprising: (a) selecting for an immunotherapy a patient having a cancer wherein the patient does not comprise a loss-of-function TP53 mutation, and (b) administering to that patient the immunotherapy. A further embodiment provides a method of determining responsiveness of a cancer to an immunotherapy, comprising detecting a presence or an absence of a TP53 loss-of-function mutation, wherein the presence of a TP53 loss-of-function mutation indicates a reduced likelihood of response of the cancer to the immunotherapy, and the absence of a TP53 loss-of-function mutation indicates an increased likelihood of response of the cancer to the immunotherapy. In some embodiments, the immunotherapy comprises administration of one or more immune checkpoint inhibitors, adoptive T-cell therapy, dendritic cell vaccination, or any combinations thereof.

In some embodiments, the immune checkpoint regulator comprises an immune checkpoint inhibitor or an immune checkpoint activator. In some embodiments, the immune checkpoint activator is an agonist of costimulation by CD27, CD40, OX40, GITR, CD137, CD28, or ICOS. In some embodiments, the checkpoint activator is an agonist antibody that binds to CD27, CD40, OX40, GITR, CD137, CD28, or ICOS. In some embodiments, the immune checkpoint inhibitor is an antagonist of PD-1, PD-L1, CTLA-4, A2AR, B7-H3, B7-H4, BTLA, IDO, KIR, LAG3, TIM-3, VISTA, CD160, TIGIT or PSGL-1. In some embodiments, the immune checkpoint inhibitor is an antagonist antibody that binds to PD-1, PD-L1, CTLA-4, A2AR, B7-H3, B7-H4, BTLA, IDO, KIR, LAG3, TIM-3, VISTA, CD160, TIGIT or PSGL-1. In some embodiments, the cancer comprises a solid tumor, lymphoma or leukemia. In some embodiments, the cancer is medulloblastoma. In some embodiments, the loss-of-function TP53 mutation is detected by DNA sequencing of TP53 gene isolated from a biological sample obtained from the patient, by measuring the expression of TP53 protein in the biological sample, or by RNA expression analysis of TP53 target genes. In some embodiments, the TP53 target genes comprise ERAP1 and TAP1. In some embodiments, the immunotherapy is administered in combination with a further therapy. In some embodiments, said further therapy comprises administering radiation, surgery, hormonal agents, or combinations thereof. In some embodiments, the loss-of-function TP53 mutation comprises substitution or deletion of one or more nucleotides of a sequence set forth as SEQ ID NO: 1, or any combination thereof. In some embodiments, the loss-of-function TP53 mutation comprises a copy number loss of TP53. In some embodiments, the loss-of-function TP53 mutation results in inactivation of the TP53 protein. In some embodiments, the inactivation of the TP53 protein renders the TP53 protein incapable of activating its downstream targets. In some embodiments, the downstream targets comprise ERAP1 and TAP1. In some embodiments, the biological sample is a tumor sample. In some embodiments, the tumor sample is a tumor biopsy.

One embodiment provides a method of identifying a cancer patient as having an increased likelihood of response to an immunotherapy, said method comprising the steps of:
(i) obtaining a tumor sample from said patient and detecting whether the tumor sample comprises a loss-of-function TP53 mutation; and
(ii) identifying said patient as having an increased likelihood of response to the immunotherapy if the tumor sample does not comprise the loss-of-function TP53 mutation and identifying said patient as having a reduced likelihood of response to the immunotherapy if the tumor sample comprises the loss-of-function TP53 mutation.

One embodiment provides a method of identifying a cancer patient as having an increased likelihood of response to an immunotherapy, said method comprising the steps of:
(i) obtaining a tumor sample from said patient and assaying levels of ERAP1 and TAP1 in said tumor sample; and
(ii) identifying said patient as having an increased likelihood of response to the immunotherapy if the levels of ERAP1 or TAP1, or both, in the tumor sample is comparable to that in a reference non-tumor biological sample and identifying said patient as having a reduced likelihood of response to the immunotherapy if the levels of ERAP1 or TAP1, or both, are lower in the tumor sample than in the reference non-tumor biological sample.

In some embodiments, the method further comprises assaying a level of MHC-I in the tumor sample and identifying said patient as having an increased likelihood of response to the immunotherapy if the level of MHC-I protein is comparable to that in the reference non-tumor biological sample and identifying said patient as having a reduced likelihood of response to the immunotherapy if the level of MHC-I is lower in the tumor sample than in the reference non-tumor biological sample.

One embodiment provides a method of identifying a cancer patient as having an increased likelihood of response to an immunotherapy, said method comprising the steps of:
(i) obtaining a tumor sample from said patient and assaying a level of MHC-I in said tumor sample; and
(ii) identifying said patient as having an increased likelihood of response to the immunotherapy if the level of the MHC-I protein in the tumor sample is comparable to that in a reference non-tumor biological sample and identifying said patient as having a reduced likelihood of response to the immunotherapy if the MHC-I level is lower in the tumor sample than in the reference non-tumor biological sample.

In some embodiments, the method further comprises assaying levels of ERAP1 and TAP1 in the tumor sample and identifying said patient as having an increased likelihood of response to the immunotherapy if the levels ERAP1 and TAP1, or both, are comparable to that in the reference non-tumor biological sample and identifying said patient as having a reduced likelihood of response to the immunotherapy if the levels of ERAP1 and TAP1, or both, are lower in the tumor sample than in the reference non-tumor biological sample.

One embodiment provides a method of identifying a cancer patient as having an increased likelihood of response to an immunotherapy, said method comprising the steps of:
(i) obtaining a tumor sample from said patient and performing the following steps:
   a) detecting whether the tumor sample comprises a loss-of-function TP53 mutation, and
   b) assaying a level of at least one of MHC-I, ERAP1, and TAP1 in said tumor sample; and
(ii) identifying said patient as having an increased likelihood of response to the immunotherapy if the tumor sample does not comprise the loss-of-function TP53 mutation or if the level of at least one of MHC class 1, ERAP1, and TAP1 in the tumor sample is comparable to that in a reference non-tumor biological sample and identifying said patient has having a reduced likelihood of response to the immunotherapy if the tumor sample comprises a loss-of-function TP53 mutation or if the level of at least one of MHC class 1, ERAP1, and TAP1 in the tumor sample is lower than that in a reference non-tumor biological sample.

In some embodiments, the method comprises detecting whether the tumor sample comprises the loss-of-function TP53 mutation prior to assaying the level of at least one of MHC-I, ERAP1, and TAP1 in the tumor sample. In some embodiments, the method comprises assaying the level of at least one of MHC-I, ERAP1, and TAP1 in the tumor sample prior to detecting whether the tumor sample comprises the loss-of-function TP53 mutation. In some embodiments, the reference non-tumor biological sample is isolated from the same patient. In some embodiments, the immunotherapy is not administered to the patient identified as having the reduced likelihood of response, thereby avoiding immunotherapy related side effects in said patient. In some embodiments, the method further comprises administering the immunotherapy to the patient identified as having the increased likelihood of response. In some embodiments, the method further comprises administering a therapy comprising TNF-α to the patient identified as having the reduced likelihood of response. In some embodiments, the immunotherapy involves T-cell based recognition of MHC-I. In some embodiments, the immunotherapy comprises administration of one or more immune checkpoint regulators, adoptive T-cell therapy, dendritic cell vaccination, or combinations thereof In some embodiments, the immune checkpoint regulator comprises an immune checkpoint inhibitor or an immune checkpoint activator. In some embodiments, the immune checkpoint activator is an agonist of costimulation by CD27, CD40, OX40, GITR, CD137, CD28, or ICOS. In some embodiments, the immune checkpoint activator is an agonist antibody that binds to CD27, CD40, OX40, GITR, CD137, CD28, or ICOS. In some embodiments, the immune checkpoint inhibitor is an antagonist of PD-1, PD-L1, CTLA-4, A2AR, B7-H3, B7-H4, BTLA, IDO, KIR, LAG3, TIM-3, VISTA, CD160, TIGIT or PSGL-1. In some embodiments, the immune checkpoint inhibitor is an antagonist antibody that binds to PD-1, PD-L1, CTLA-4, A2AR, B7-H3, B7-H4, BTLA, IDO, KIR, LAG3, TIM-3, VISTA, CD160, TIGIT or PSGL-1. In some embodiments, the cancer comprises a solid tumor, lymphoma, or leukemia. In some embodiments, the cancer is medulloblastoma.

In some embodiments, the detection is carried out by DNA sequencing of TP53 gene isolated from the biological sample, by measuring the expression of TP53 protein in the biological sample, or by RNA expression analysis of TP53 target genes. In some embodiments, the TP53 target genes comprise ERAP1 and TAP1. In some embodiments, identifying a patient as having a reduced likelihood of response to the immunotherapy reduces the risk of side effects associated with administering the immunotherapy to the patient without any therapeutic benefit.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which.

FIG. 1 shows a schematic illustration of the regulation of class 1 MHC molecule (MHC-I) by TP53.

FIG. 2 shows that medulloblastoma tumor cells infected with viruses encoding Myc and a dominant negative form of TP53 (MP tumors) or Myc and the transcriptional repressor Gfi1 (MG tumors) display distinct growth patterns in immunocompetent mice. FIG. 2(A) shows that MP tumors grow in immunocompromised (NSG) and immunocompetent (B6) mice and FIG. 2(B) shows that MG tumor only grow in immunocompromised (NSG) mice.

FIG. 3 shows that loss of TP53 leads to downregulation of MHC-I on medulloblastoma tumor cells.

FIG. 4 shows that loss of TP53 inhibits expression of ERAP1 and TAP1 RNA in medulloblastoma tumor cells.

FIG. 5 shows that TP53-mediated MHC regulation also occurs in pancreatic cancer. PanIN cells (with wild type TP53; top) express more MHC-I than do pancreatic cancer cells (with deleted TP53; bottom). Summary of data is shown in the bar graph on right.

FIG. 6 shows that TP53-mutations are associated with reduced levels of ERAP1 in human breast cancer FIG. 6(A), colon cancer FIG. 6 (B) and acute myeloid leukemia FIG. 6 (C), based on analysis of The Cancer Genome Atlas (TCGA).

FIG. 7 shows that medulloblastoma tumors with different TP53 function (MP tumors and MG tumors) display distinct growth patterns after transplantation into mice. FIGS. 7(A) and 7(B) show that MP tumors grew in immunocompromised (NSG; thinner line on the survival curve shown in FIG. 7(B)) and immunocompetent (aB6; thicker line on the survival curve shown in FIG. 7(B)) mice and FIGS. 7(C) and 7(D) show that only 4.4% of mice transplanted with MG tumor cells developed tumors, in particular, FIGS. 7(C) and 7(D) show that the MG tumors were only able to grow efficiently in immunocompromised mice (NSG; thinner line on the survival curve shown in FIG. 7(C)) and not in immunocompetent mice (aB6; thicker line of the survival curve shown in FIG. 7(C)). FIGS. 7(E), 7(F), and 7(G) show that the depletion of CD4+ (helper) or CD8+ (cytotoxic) T cells allowed MG tumors to grow in immunocompetent mice (bioluminescence images of representative mice are shown in FIG. 7(E). FIG. 7(F) shows quantification of average bioluminescence signal from various groups of mice. FIG. 7(G) shows survival curves. FIG. 8 shows the relationship between tumor growth and MHC-I expression. FIGS. 8(A) and 8(B) show that transducing MG tumors with DNp53 (MG+P) had a dramatic effect on MG tumors, allowing them to grow in immunocompetent mice (aB6). FIG. 8(A) shows bioluminescence images of representative mice and FIG. 8(B) shows survival curve. FIG. 8(C) shows an FACS (fluorescence activated cell sorting) histogram indicating that MG tumors (solid line with dotted fill) expressed significant amounts of MHC-I on the cell surface, while MP tumors (solid line with no fill) expressed virtually none, compared to isotype control for MP (dashed line with no fill). FIG. 8(D) shows an FACS histogram demonstrating that MG tumors transduced with a dominant negative form of TP53, MG+P (dashed line with no fill) showed a marked downregulation of MHC-I compared to MG tumors (solid line with dotted fill). FIGS. 8(E) (bioluminescence images of representative mice) and 8(F) (survival curves) show that MG tumors lacking MHC-I are able to grow in immunocompetent mice (MG MHC-1 KO aB6). FIGS. 8(G) and 8(H) show that MP tumor cells only have markedly decreased cell surface MHC-I but no difference in the levels of MHC-I mRNA or total cellular MHC-protein. FIG. 8(G) shows analysis of MHC class I (MHCk(b)) and (MHCd(b)) determined by RT-qPCR. FIG. 8(H) shows protein levels of MHC class I and Actin, determined by western blotting.

FIG. 9 shows that Erap1 and Tap1 are associated with the cell surface localization of MHC-I. FIGS. 9(A), 9(B), and 9(C) show that MP tumors express significantly less Tap1 and Erap1 than MG tumor cells. FIG. 9(A) shows mRNA expression levels for Tap1, FIG. 9(B) shows mRNA expression levels for Erap1, and FIG. 9(C) shows western blotting results. for FIGS. 9(D) and 9(E) show that human MB samples of tumors with TP53 mutations have a lower expression of TAP1 and ERAP1 than wild type TP53, as indicated by mRNA expression levels. FIGS. 9(F) and 9(G) show a decrease in MHC-I expression in MG tumor cells following shRNA-mediated knockdown of Erap1. FIG. 9(G) shows an FACS histogram of MG tumor cells transduced with shRNA (shCtl-solid line with dotted fill) or shErap1 (dashed line with no fill). FIGS. 9(H) (bioluminescence images of representative mice) and 9(I) (survival curves) show that Erap1 knockdown allowed MG tumors to grow in immunocompetent mice (shErap1#1 aB6 and shErap1#2 aB6), as compared to transduction with shRNA (shCtl), p-value for the difference in survival between shErap1 and shCtl was determined by the log-rank (Mantel-Cox) test. FIGS. 9(J) and 9(K) show that the overexpression of Erap1 in MP tumor cells resulted in increased MHC-I expression, as compared to empty vector (in the FACS histogram of FIG. 9(K), Erap1 overexpressing MP tumors are represented by a solid line with no fill and empty vector is shown as dotted line with no fill). FIGS. 9(L) (bioluminescence images of representative mice) and 9(M) (survival curves) show that overexpression of Erap1 and Tap1 in MP tumor cells prolonged survival of tumor-bearing mice.

FIG. 10 shows that TNF-α can be administered safely and can increase the expression of MHC-I in tumor cells in vitro and in vivo. The FACS histograms of FIGS. 10(A) and 10(B) show that treatment with IFNγ increases expression of MHC-I in MG tumors (FIG. 10(A): ctl-dashed line with no fill; IFNγ-solid line with dotted fill), which already expresses MHC-I, but does not increase MHC-I expression in MP tumors (FIG. 10(B): ctl-dashed line with no fill; IFNγ-solid line with dotted fill). FIGS. 10(C) and 10(D) show that TNF-α caused a marked increase in MHC-I expression in both MG (FIG. 10(C): ctl-dashed line with no fill; TNFα-solid line with dotted fill) and MP tumors (FIG. 10(D): ctl-dashed line with no fill; TNFα-solid line with dotted fill). FIGS. 10(E)-10(G) show that TNF-α, but not IFNγ, increased the expression of Erap1 (FIG. 10(E)-mRNA expression levels) and Tap1 (FIG. 10(F)-mRNA expression levels) in MP tumor cells. FIG. 10(G) shows western blotting results. FIG. 10(H) shows the marked increase in MHC-I expression after TNF-α IV treatment. FIGS. 10(I) (bioluminescence images of representative mice), 10(J) (quantification of bioluminescence signal from each group), and 10(K) (survival curves) show that a combination of anti-PD-1+TNF-α markedly inhibit tumor growth and increase survival rates.

FIG. 11 shows that MP tumors and MG tumors display distinct growth patterns, and that this is not due to Gfi1. FIGS. 11(A) (bioluminescence images of representative mice) and 11(B) (survival curves) show that Gfi1 had no effect on the growth of MP tumors. FIGS. 11(C) and 11(D) show that the expression of molecules known to regulate immune responses (expression of T cell suppression molecules shown in FIG. 11(C) and expression of dendritic cell and T cell activation markers are shown in FIG. 11(D)) did not differ between MP and MG tumors.

FIG. 12 shows the relationship between TP53 function and the expression of cell surface MHC-I in a variety of different medulloblastoma cells. FIG. 12(A) shows the downregulation of MHC-I following the overexpression of a dominant negative form of TP53 in murine Patched-knockout tumors, a model of Sonic hedgehog-associated medulloblastoma (control-dashed line with no fill; DNP53-solid line with no fill). FIGS. 12(B) (control empty vector-dashed line with no fill; shp53-solid line with no fill and solid line with dotted fill) and 12(C) (control empty vector-dashed line with no fill; shp53-solid line with no fill and solid line with dotted fill) show the decreased expression of MHC-I following the shRNA-mediated knockdown of TP53 in murine MG tumors and in the human medulloblastoma cell line HD-MB03. FIG. 12(D) shows the decreased expression of MHC-I (HLA-I) in medulloblastoma patient-derived xenografts (PDXs) with TP53 mutations (upper panel-TP53 mutated PDX) (HLA-1 staining-solid line with no fill; isotype control-dashed line with no fill) compared to PDXs without TP53 mutations (lower panel-TP53 WT PDX) (HLA-1 staining-solid line with no fill; isotype control-dashed line with no fill).

FIG. 13 shows the relationship between Tap1 and the expression of MHC-I. FIGS. 13(A) (western blot) and 13(B) (FACS histogram; shTap1-solid line with no fill, shCtl-dashed line with no fill) show the knockdown of Tap1 decreased MHC-I expression in MG tumor cells. FIGS. 13(C) (bioluminescence images of representative mice) and 13(D) (survival curves) show that the knockdown of Tap1 in MG tumor cells resulted in a longer latency of tumor growth in syngeneic mice (shTap#1 aB6 and shTap2#2 aB6). FIGS. 13(E) (western blot) and 13(F) (FACS histogram; empty vector-dashed line with no fill; Tap1-solid line with no fill) show the overexpression of Tap1 only modestly affected MHC-I expression

FIG. 14 shows that TNF-α and LTβ receptor agonist can increase the expression of HLA-I. FIG. 14(A) (Control-dashed line with no fill; TNFα-solid line with no fill) shows the increase in HLA-I expression with the addition of TNF-α in TP53-mutant (upper panel) and TP53-WT (lower panel) medulloblastoma PDXs. FIGS. 14(B) and 14(C) show the addition of LTβ receptor agonist increases MHC-I expression in MG (FIG. 14(B): control-dashed line with no fill; LTβRag-solid line with no fill) and MP (FIG. 14(C): control-dashed line with no fill; LTβRag-solid line with no fill) tumor cells. FIGS. 14(D) and 14(E) show the increase in Tap1 and Erap1 mRNA expression with addition of LTβ receptor agonist and FIG. 14(F) shows the increase in MHC-I expression following treatment of tumor-bearing mice with LTβ receptor agonist (control-dashed line with no fill; LTβRag-solid line with no fill). FIG. 14(G) shows that no toxicity is seen after low doses of TNF-α. FIGS. 14(H) (quantification of the average bioluminescence signal for each group) and 14(I) (survival curves) show that the use of TNF-α to sensitize tumor cells is dependent on the expression of MHC-I.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).The terms “heterologous nucleic acid sequence,” as used herein, in relation to a specific virus refers to a nucleic acid sequence that originates from a source other than the specified virus.

The term “mutation,” as used herein, refers to a deletion, an insertion of a heterologous nucleic acid, an inversion or a substitution, including an open reading frame ablating mutations as commonly understood in the art.

The term “gene,” as used herein, refers to a segment of nucleic acid that encodes an individual protein or RNA (also referred to as a “coding sequence” or “coding region”), optionally together with associated regulatory regions such as promoters, operators, terminators and the like, which may be located upstream or downstream of the coding sequence.

The term “homology,” as used herein, may be to calculations of “homology” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=# of identical positions/total # of positions×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90-5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g. , W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

The terms “treat,” “treating,” and “treatment” is meant to include alleviating or abrogating a disorder, disease, or condition; or one or more of the symptoms associated with the disorder, disease, or condition; or alleviating or eradicating the cause(s) of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refer to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

An “anti-cancer agent,” as used herein, can refer to an agent or therapy that is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Non-limiting examples of anti-cancer agents can include biological agents (biotherapy), chemotherapy agents, and radiotherapy agents.

### TP53 Mutations and Immunotherapy

Embodiments of the present disclosure relate to methods of identifying cancer patients as having an increased or reduced likelihood of responding to a therapy, by identifying TP53 gene status in biological samples isolated from said cancer patients. In certain instances, the TP53 gene status is a function of a presence or an absence of a loss-of-function TP53 mutation. In some embodiments, the cancer patient is identified as having an increased likelihood of responding to the therapy if the biological sample isolated from the patient does not contain a loss-of-function TP53 mutation and a reduced likelihood of responding to the therapy if the biological sample contains a loss-of-function TP53 mutation.

Further provided are methods of treating a cancer patient by selecting a patient who does not have a loss-of-function TP53 mutation and administering a therapy to the selected patient.

An additional embodiment provides a method of determining responsiveness of a cancer or a tumor to a therapy by determining TP53 gene status in said cancer or tumor. In some instances, the TP53 gene status is a function of a presence or an absence of a loss-of-function TP53 mutation. Therefore, in certain embodiments, the responsiveness of the cancer or the tumor to the therapy is determined by detecting the presence or the absence of the loss-of-function TP53 mutation.

The therapy, in any of the above embodiments, is an immunotherapy, alone or in combination with an additional anti-cancer treatment, such as chemotherapy, radiation, surgery, hormonal agents, or any combinations thereof.

TP53 gene status is, in certain examples, detected in tumor samples or in biological samples such as blood, urine, stool, sputum or serum. For example, TP53 mutations are often detected in urine for bladder cancer and prostate cancer, sputum for lung cancer, or stool for colorectal cancer. Serum is mostly tested in the context of colorectal cancer, however serum analysis should work for any tumor type that sheds cancer cells into the blood. Cancer cells are found in blood and serum for cancers such as lymphoma or leukemia. The same techniques discussed above for detection of mutant p53 genes or gene products in tumor samples can be applied to other body samples. Cancer cells are sloughed off from tumors and appear in such body samples. The TP53 gene status is identified, for example, using techniques such as sequencing of TP53 gene, RNA expression analysis of TP53 or its target genes, e.g., TAP1 and ERAP1, assaying the level of p53 protein, coded by the TP53 gene, or its downstream target proteins, e.g., TAP1 and ERAP 1, or quantitative PCR, or by assaying the level of MHC-I.

### Loss-of-Function TP53 Mutation

In some embodiments, a loss-of-functionTP53 mutation is an inactivating missense mutation in one allele and simultaneous deletions in regions of the 17p of the chromosome encompassing the TP53 locus. The loss-of-functionTP53 mutation is, in some examples, a point mutation, such as a missense mutation, a nonsense mutation, a frameshift mutation, or a deletion mutation (which results in reduction in TP53 copy number), or any combinations thereof. For instance, in some embodiments, the loss-of-function TP53 mutation is a missense mutation together with a segmental 17p deletion. In other embodiments, the loss-of-function TP53 mutation is only a 17p deletion together with wild-type TP53 allele. In some instances, the loss-of-function TP53 mutation is a deletion on chromosome 17p13, also referred to herein as 17p13 deletion.

In some embodiments, an alternate isoform of p53, produced by alternative splicing of the TP53 gene, is associated with the increased or reduced likelihood of a cancer patient responding to an immunotherapy. Non-limiting examples of p53 isoforms include, p53-beta and p53-gamma isoforms which are produced by intron-9, Δ40p53-alpha, Δ40p53-beta, Δ40p53-gamma isoforms which are generated by the alternative splicing of intron-2.

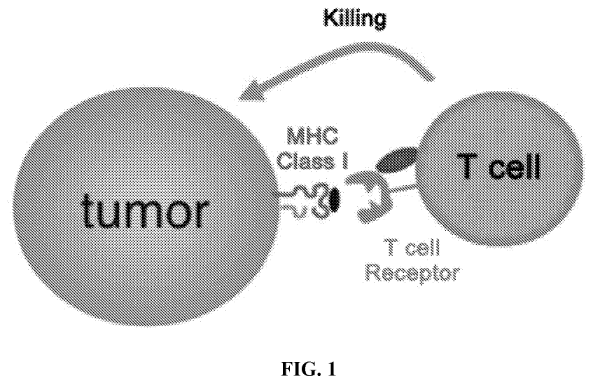

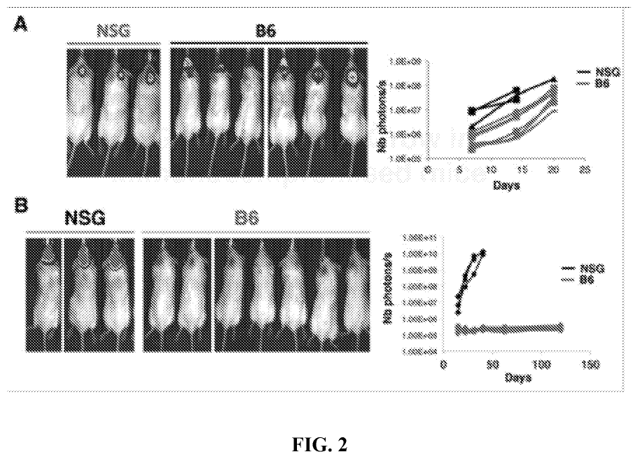

In some embodiments, a loss-of-function TP53 mutation that is correlated to a reduced likelihood of a cancer patient responding to an immunotherapy is within exons 4-9 of the TP53 gene. In some embodiments, a loss-of-function TP53 mutation that is correlated to a reduced likelihood of a cancer patient responding to an immunotherapy is within the nucleotide residues coding for amino acid positions R175, G245, R248, R249, R273, and R282 of a human TP53 protein, comprising a sequence as set forth in SEQ ID NO: 1. See also, FIG. 1 which depicts the structure of the TP53 gene. In some embodiments, a loss-of-function TP53 mutation that is correlated to a reduced likelihood of a cancer patient responding to an immunotherapy is a TP53 truncating mutation that occurs at the boundary of exons 6 and 7. A missense TP53 mutation is, in some examples, a single-nucleotide substitutions (SNS) that cluster within the DNA-binding domain of the protein.

Non-limiting examples of TP53 mutations are provided in Table 1. Genomic and gene variant data referred to in Table 1 is, in various cases, obtained from Life Technologies and Compendia Bioscience's ONCOMINE™ Concepts Edition and ONCOMINE™ Power Tools, a suite of web applications and web browsers that integrates and unifies high-throughput cancer profiling data by systematic collection, curation, ontologization and analysis. In addition, mutation data is derived from sources such as Sanger Institute's Catalogue of Somatic Mutations in Cancer (COSMIC). Original annotation is retained for mutation data from COSMIC. Accession numbers listed in Table 1 are Reference Sequence (RefSeq) accession numbers for the corresponding NCBI Reference Sequence. The CDS and amino acid mutation syntax show standard mutation nomenclature based on coding DNA reference sequence and amino acid sequence, respectively (e.g., the naming standard recommended by the Human Genome Variation Society, as described at http://www.hgvs.org/mutnomen/).

TABLE 1
Non-limiting examples of TP53 mutations.

| | COSMIC | | Amino Acid- | Oncomine gene | Oncomine variant |
|---|---|---|---|---|---|
| Accession No. | ID | CDS-mutation-syntax | mutation-syntax | classification | classification |
| NM_000546 | 18657 | c.560 − 2A > G | p.? | Loss of Function | N/A |
| NM_000546 | 21572 | c.376 − 1G > A | p.? | Loss of Function | N/A |
| NM_000546 | 22908 | c.376 − 1G > T | p.? | Loss of Function | N/A |
| NM_000546 | 43541 | c.559 + 3G > C | p.? | Loss of Function | N/A |
| NM_000546 | 43753 | c.560 − 1G > A | p.? | Loss of Function | N/A |
| NM_000546 | 43841 | c.560 − 1G > T | p.? | Loss of Function | N/A |
| NM_000546 | 43872 | c.560 − 1G > C | p.? | Loss of Function | N/A |
| NM_000546 | 43927 | c.559 + 9C > T | p.? | Loss of Function | N/A |
| NM_000546 | 44268 | c.559 + 1G > T | p.? | Loss of Function | N/A |
| NM_000546 | 44297 | c.376 − 3C > T | p.? | Loss of Function | N/A |
| NM_000546 | 44495 | c.559 + 2T > A | p.? | Loss of Function | N/A |
| NM_000546 | 44933 | c.376 − 4A > G | p.? | Loss of Function | N/A |
| NM_000546 | 45026 | c.560 − 2A > T | p.? | Loss of Function | N/A |
| NM_000546 | 45364 | c.376 − 1delG | p.? | Loss of Function | N/A |
| NM_000546 | 45672 | c.376 − 2A > G | p.? | Loss of Function | N/A |
| NM_000546 | 45711 | c.559 + 2T > G | p.? | Loss of Function | N/A |
| NM_000546 | 45809 | c.376 − 1G > C | p.? | Loss of Function | N/A |
| NM_000546 | 46049 | c.376 − 2A > C | p.? | Loss of Function | N/A |
| NM_000546 | 46059 | c.560 − 3T > G | p.? | Loss of Function | N/A |
| NM_000546 | 6900 | c.376 − 1G > A | p.? | Loss of Function | N/A |
| NM_000546 | 6901 | c.559 + 1G > A | p.? | Loss of Function | N/A |
| NM_000546 | 44966 | c.385G > A | p.A129T | Loss of Function | Missense_Mutation |
| NM_000546 | 44550 | c.386C > T | p.A129V | Loss of Function | Missense_Mutation |
| NM_000546 | 44130 | c.477C > T | p.A159A | Loss of Function | Synonymous_Mutation |
| NM_000546 | 11496 | c.476C > A | p.A159D | Loss of Function | Missense_Mutation |
| NM_000546 | 45057 | c.475delG | p.A159fs*11 | Loss of Function | Frame_Shift_Del |
| NM_000546 | 43836 | c.475G > C | p.A159P | Loss of Function | Missense_Mutation |
| NM_000546 | 45286 | c.475G > T | p.A159S | Loss of Function | Missense_Mutation |
| NM_000546 | 43626 | c.475G > A | p.A159T | Loss of Function | Missense_Mutation |
| NM_000546 | 11148 | c.476C > T | p.A159V | Loss of Function | Missense_Mutation |
| NM_000546 | 44119 | c.483C > T | p.A161A | Loss of Function | Synonymous_Mutation |
| NM_000546 | 11323 | c.482C > A | p.A161D | Loss of Function | Missense_Mutation |
| NM_000546 | 44230 | c.481delG | p.A161fs*9 | Loss of Function | Frame_Shift_Del |
| NM_000546 | 10739 | c.481G > A | p.A161T | Loss of Function | Missense_Mutation |
| NM_000546 | 43689 | c.482C > T | p.A161V | Loss of Function | Missense_Mutation |
| NM_000546 | 45029 | c.565_591del27 | p.A189_V197delAPP | Loss of Function | In_Frame_Del |
| NM_000546 | 45440 | c.567C > T | p.A189A | Loss of Function | Synonymous_Mutation |
| NM_000546 | 43698 | c.566C > G | P.A189G | Loss of Function | Missense_Mutation |
| NM_000546 | 44923 | c.565G > C | p.A189P | Loss of Function | Missense_Mutation |
| NM_000546 | 43537 | c.565G > A | p.A189T | Loss of Function | Missense_Mutation |
| NM_000546 | 44349 | c.566C > T | p.A189V | Loss of Function | Missense_Mutation |
| NM_000546 | 45268 | c.827C > A | p.A276D | Loss of Function | Missense_Mutation |
| NM_000546 | 45695 | c.827C > G | p.A276G | Loss of Function | Missense_Mutation |
| NM_000546 | 43663 | c.826G > C | p.A276P | Loss of Function | Missense_Mutation |
| NM_000546 | 45467 | c.826G > T | p.A276S | Loss of Function | Missense_Mutation |
| NM_000546 | 44114 | c.826G > A | p.A276T | Loss of Function | Missense_Mutation |
| NM_000546 | 10756 | c.827C > T | p.A276V | Loss of Function | Missense_Mutation |
| NM_000546 | 44019 | c.226_270del45 | p.A76_S90del15 | Loss of Function | In_Frame_Del |
| NM_000546 | 45200 | c.233C > T | p.A78V | Loss of Function | Missense_Mutation |
| NM_000546 | 44075 | c.251C > G | p.A84G | Loss of Function | Missense_Mutation |
| NM_000546 | 44194 | c.251C > T | p.A84V | Loss of Function | Missense_Mutation |
| NM_000546 | 44231 | c.262delG | p.A88fs*35 | Loss of Function | Frame_Shift_Del |
| NM_000546 | 44319 | c.405C > A | p.C135* | Loss of Function | Nonsense_Mutation |
| NM_000546 | 43704 | c.405C > T | p.C135C | Loss of Function | Synonymous_Mutation |
| NM_000546 | 10647 | c.404G > T | p.C135F | Loss of Function | Missense_Mutation |
| NM_000546 | 44670 | c.400delT | p.C135fs*35 | Loss of Function | Frame_Shift_Del |
| NM_000546 | 44829 | c.403T > G | p.C135G | Loss of Function | Missense_Mutation |
| NM_000546 | 10684 | c.403T > C | p.C135R | Loss of Function | Missense_Mutation |
| NM_000546 | 44643 | c.404G > C | p.C135S | Loss of Function | Missense_Mutation |
| NM_000546 | 44910 | c.403T > A | p.C135S | Loss of Function | Missense_Mutation |
| NM_000546 | 44219 | c.405C > G | p.C135W | Loss of Function | Missense_Mutation |
| NM_000546 | 10801 | c.404G > A | p.C135Y | Loss of Function | Missense_Mutation |
| NM_000546 | 43734 | c.528C > A | p.C176* | Loss of Function | Nonsense_Mutation |
| NM_000546 | 45399 | c.526_543del18 | p.C176_R181delCPH | Loss of Function | In_Frame_Del |
| NM_000546 | 10645 | c.527G > T | p.C176F | Loss of Function | Missense_Mutation |
| NM_000546 | 44759 | c.526delT | p.C176fs*71 | Loss of Function | Frame_Shift_Del |
| ENST00000269305 | 99601 | c.404G > A | p.C135Y | Loss of Function | Missense_Mutation |
| NM_000546 | 44948 | c.526T > C | p.C176R | Loss of Function | Missense_Mutation |
| NM_000546 | 44146 | c.526T > A | p.C176S | Loss of Function | Missense_Mutation |
| ENST00000413465 | 99598 | c.404G > A | p.C135Y | Loss of Function | Missense_Mutation |
| ENST00000545858 | 99625 | c.434G > T | p.C145F | Loss of Function | Missense_Mutation |
| NM_000546 | 10687 | c.527G > A | p.C176Y | Loss of Function | Missense_Mutation |
| NM_000546 | 45562 | c.546C > A | p.C182* | Loss of Function | Nonsense_Mutation |
| ENST00000269305 | 117398 | c.527G > T | p.C176F | Loss of Function | Missense_Mutation |
| ENST00000413465 | 117395 | c.527G > T | p.C176F | Loss of Function | Missense_Mutation |
| NM_000546 | 44692 | c.526T > G | p.C176G | Loss of Function | Missense_Mutation |
| NM_000546 | 44645 | c.527G > C | p.C176S | Loss of Function | Missense_Mutation |
| NM_000546 | 45394 | c.687T > A | p.C229* | Loss of Function | Nonsense Mutation |
| NM_000546 | 45654 | c.685_699del15 | p.C229_H233delCTT | Loss of Function | In_Frame_Del |
| NM_000546 | 43648 | c.685_686delTG | p.C229fs*10 | Loss of Function | Frame_Shift_Del |
| NM_000546 | 44360 | c.686_687delGT | p.C229fs*10 | Loss of Function | Frame_Shift_Del |
| NM_000546 | 11114 | c.528C > G | p.C176W | Loss of Function | Missense_Mutation |
| NM_000546 | 46288 | c.546C > T | p.C182C | Loss of Function | Synonymous_Mutation |
| NM_000546 | 45677 | c.714T > A | p.C238* | Loss of Function | Nonsense_Mutation |
| NM_000546 | 43778 | c.713G > T | p.C238F | Loss of Function | Missense_Mutation |
| NM_000546 | 44563 | c.544T > C | p.C182R | Loss of Function | Missense_Mutation |
| NM_000546 | 43828 | c.544T > A | p.C182S | Loss of Function | Missense_Mutation |
| NM_000546 | 43700 | c.712T > A | p.C238S | Loss of Function | Missense_Mutation |
| NM_000546 | 44653 | c.713G > C | p.C238S | Loss of Function | Missense_Mutation |
| NM_000546 | 44676 | c.714T > G | p.C238W | Loss of Function | Missense_Mutation |
| NM_000546 | 11059 | c.713G > A | p.C238Y | Loss of Function | Missense_Mutation |
| NM_000546 | 44378 | c.726C > A | p.C242* | Loss of Function | Nonsense_Mutation |
| NM_000546 | 45691 | c.726C > T | p.C242C | Loss of Function | Synonymous_Mutation |
| NM_000546 | 10810 | c.725G > T | p.C242F | Loss of Function | Missense_Mutation |
| NM_000546 | 44657 | c.722delC | p.C242fs*5 | Loss of Function | Frame_Shift_Del |
| NM_000546 | 6530 | c.723delC | p.C242fs*5 | Loss of Function | Frame_Shift_Del |
| NM_000546 | 44546 | c.545G > A | p.C182Y | Loss of Function | Missense_Mutation |
| NM_000546 | 45612 | c.685T > C | p.C229R | Loss of Function | Missense_Mutation |
| NM_000546 | 11133 | c.725G > C | p.C242S | Loss of Function | Missense_Mutation |
| NM_000546 | 44935 | c.724T > A | p.C242S | Loss of Function | Missense_Mutation |
| NM_000546 | 44313 | c.686G > A | p.C229Y | Loss of Function | Missense_Mutation |
| NM_000546 | 10646 | c.725G > A | p.C242Y | Loss of Function | Missense_Mutation |
| NM_000546 | 44735 | c.825T > C | p.C275C | Loss of Function | Synonymous_Mutation |
| NM_000546 | 10701 | c.824G > T | p.C275F | Loss of Function | Missense_Mutation |
| ENST00000269305 | 99626 | c.713G > T | p.C238F | Loss of Function | Missense_Mutation |
| ENST00000413465 | 99624 | c.713G > T | p.C238F | Loss of Function | Missense_Mutation |
| NM_000546 | 45413 | c.824G > C | p.C275S | Loss of Function | Missense_Mutation |
| NM_000546 | 46336 | c.712T > G | p.C238G | Loss of Function | Missense_Mutation |
| NM_000546 | 10893 | c.824G > A | p.C275Y | Loss of Function | Missense_Mutation |
| NM_000546 | 44972 | c.831T > A | p.C277* | Loss of Function | Nonsense_Mutation |
| NM_000546 | 45109 | c.831T > C | p.C277C | Loss of Function | Synonymous_Mutation |
| NM_000546 | 10749 | c.830G > T | p.C277F | Loss of Function | Missense_Mutation |
| NM_000546 | 44321 | c.712T > C | p.C238R | Loss of Function | Missense_Mutation |
| NM_000546 | 44135 | c.724T > G | p.C242G | Loss of Function | Missense_Mutation |
| NM_000546 | 11738 | c.724T > C | p.C242R | Loss of Function | Missense_Mutation |
| NM_000546 | 45338 | c.552T > C | p.D184D | Loss of Function | Synonymous_Mutation |
| NM_000546 | 11356 | c.726C > G | p.C242W | Loss of Function | Missense_Mutation |
| ENST00000269305 | 99932 | c.824G > T | p.C275F | Loss of Function | Missense_Mutation |
| NM_000546 | 11501 | c.823T > G | p.C275G | Loss of Function | Missense_Mutation |
| NM_000546 | 45823 | c.558T > C | p.D186D | Loss of Function | Synonymous_Mutation |
| NM_000546 | 45838 | c.556delG | p.D186fs*61 | Loss of Function | Frame_Shift_Del |
| NM_000546 | 43902 | c.823T > C | p.C275R | Loss of Function | Missense_Mutation |
| NM_000546 | 43823 | c.825T > G | p.C275W | Loss of Function | Missense_Mutation |
| ENST00000269305 | 165084 | c.824G > A | p.C275Y | Loss of Function | Missense_Mutation |
| NM_000546 | 45074 | c.829T > G | p.C277G | Loss of Function | Missense_Mutation |
| NM_000546 | 45299 | c.831T > G | p.C277W | Loss of Function | Missense_Mutation |
| NM_000546 | 45796 | c.623A > G | p.D208G | Loss of Function | Missense_Mutation |
| NM_000546 | 43737 | c.830G > A | p.C277Y | Loss of Function | Missense_Mutation |
| NM_000546 | 44249 | c.623A > T | p.D208V | Loss of Function | Missense_Mutation |
| ENST00000414315 | 99599 | c.8G > A | p.C3Y | Loss of Function | Missense_Mutation |
| NM_000546 | 45028 | c.684C > T | p.D228D | Loss of Function | Synonymous_Mutation |
| ENST00000545858 | 99600 | c.125G > A | p.C42Y | Loss of Function | Missense_Mutation |
| ENST00000545858 | 117397 | c.248G > T | p.C83F | Loss of Function | Missense_Mutation |
| ENST00000414315 | 117396 | c.131G > T | p.C44F | Loss of Function | Missense_Mutation |
| NM_000546 | 43797 | c.550G > C | p.D184H | Loss of Function | Missense_Mutation |
| NM_000546 | 44029 | c.550G > A | p.D184N | Loss of Function | Missense_Mutation |
| NM_000546 | 11665 | c.842A > C | p.D281A | Loss of Function | Missense_Mutation |
| NM_000546 | 43958 | c.843C > T | p.D281D | Loss of Function | Synonymous_Mutation |
| NM_000546 | 43837 | c.843C > G | p.D281E | Loss of Function | Missense_Mutation |
| NM_000546 | 43906 | c.843C > A | p.D281E | Loss of Function | Missense_Mutation |
| NM_000546 | 44202 | c.550G > T | p.D184Y | Loss of Function | Missense_Mutation |
| NM_000546 | 10943 | c.841G > C | p.D281H | Loss of Function | Missense_Mutation |
| NM_000546 | 43596 | c.841G > A | p.D281N | Loss of Function | Missense_Mutation |
| NM_000546 | 45729 | c.842A > T | p.D281V | Loss of Function | Missense_Mutation |
| NM_000546 | 11516 | c.841G > T | p.D281Y | Loss of Function | Missense_Mutation |
| NM_000546 | 44837 | c.556G > C | p.D186H | Loss of Function | Missense_Mutation |
| NM_000546 | 10996 | c.511G > T | p.E171* | Loss of Function | Nonsense_Mutation |
| NM_000546 | 46095 | c.511delG | p.E171fs*3 | Loss of Function | Frame_Shift_Del |
| NM_000546 | 44700 | c.556G > A | p.D186N | Loss of Function | Missense_Mutation |
| NM_000546 | 44312 | c.511G > A | p.E171K | Loss of Function | Missense_Mutation |
| NM_000546 | 45519 | c.620A > G | p.D207G | Loss of Function | Missense_Mutation |
| NM_000546 | 43597 | c.538G > T | p.E180* | Loss of Function | Nonsense_Mutation |
| NM_000546 | 45372 | c.540G > T | p.E180D | Loss of Function | Missense_Mutation |
| NM_000546 | 45707 | c.624C > A | p.D208E | Loss of Function | Missense_Mutation |
| NM_000546 | 44241 | c.592G > T | p.E198* | Loss of Function | Nonsense_Mutation |
| NM_000546 | 45851 | c.624C > G | p.D208E | Loss of Function | Missense_Mutation |
| NM_000546 | 43987 | c.622G > A | p.D208N | Loss of Function | Missense_Mutation |
| NM_000546 | 10804 | c.610G > T | p.E204* | Loss of Function | Nonsense_Mutation |
| NM_000546 | 43761 | c.612G > A | p.E204E | Loss of Function | Synonymous_Mutation |
| NM_000546 | 44011 | c.610delG | p.E204fs*43 | Loss of Function | Frame_Shift_Del |
| NM_000546 | 43862 | c.683A > C | p.D228A | Loss of Function | Missense_Mutation |
| NM_000546 | 43853 | c.684C > G | p.D228E | Loss of Function | Missense_Mutation |
| NM_000546 | 44817 | c.661G > T | p.E221* | Loss of Function | Nonsense_Mutation |
| NM_000546 | 43960 | c.683A > G | p.D228G | Loss of Function | Missense_Mutation |
| NM_000546 | 44398 | c.682G > A | p.D228N | Loss of Function | Missense_Mutation |
| NM_000546 | 43750 | c.811G > T | p.E271* | Loss of Function | Nonsense_Mutation |
| NM_000546 | 45529 | c.683A > T | p.D228V | Loss of Function | Missense_Mutation |
| NM_000546 | 45786 | c.682G > T | p.D228Y | Loss of Function | Missense_Mutation |
| NM_000546 | 11232 | c.842A > G | p.D281G | Loss of Function | Missense_Mutation |
| NM_000546 | 10719 | c.811G > A | p.E271K | Loss of Function | Missense_Mutation |
| NM_000546 | 11606 | c.31G > C | p.E11Q | Loss of Function | Missense_Mutation |
| NM_000546 | 44469 | c.812A > T | p.E271V | Loss of Function | Missense_Mutation |
| NM_000546 | 44388 | c.853G > T | p.E285* | Loss of Function | Nonsense_Mutation |
| NM_000546 | 44732 | c.512A > G | p.E171G | Loss of Function | Missense_Mutation |
| NM_000546 | 44709 | c.855G > A | p.E285E | Loss of Function | Synonymous_Mutation |
| NM_000546 | 45751 | c.511G > C | p.E171Q | Loss of Function | Missense_Mutation |
| NM_000546 | 10722 | c.853G > A | p.E285K | Loss of Function | Missense_Mutation |
| NM_000546 | 43772 | c.538G > A | p.E180K | Loss of Function | Missense_Mutation |
| NM_000546 | 43690 | c.592G > A | p.E198K | Loss of Function | Missense_Mutation |
| NM_000546 | 43919 | c.856G > T | p.E286* | Loss of Function | Nonsense_Mutation |
| NM_000546 | 44292 | c.858A > G | p.E286E | Loss of Function | Synonymous_Mutation |
| NM_000546 | 44651 | c.856_863delGAAGAGAA | p.E286fs*17 | Loss of Function | Frame_Shift_Del |
| NM_000546 | 45277 | c.856delG | p.E286fs*59 | Loss of Function | Frame_Shift_Del |
| NM_000546 | 43565 | c.857A > G | p.E286G | Loss of Function | Missense_Mutation |
| NM_000546 | 10726 | c.856G > A | p.E286K | Loss of Function | Missense_Mutation |
| NM_000546 | 44250 | c.856G > C | p.E286Q | Loss of Function | Missense_Mutation |
| NM_000546 | 43936 | c.857A > T | p.E286V | Loss of Function | Missense_Mutation |
| NM_000546 | 44133 | c.859G > T | p.E287* | Loss of Function | Nonsense_Mutation |
| NM_000546 | 43776 | c.861G > A | p.E287E | Loss of Function | Synonymous_Mutation |
| NM_000546 | 45449 | c.592G > C | p.E198Q | Loss of Function | Missense_Mutation |
| NM_000546 | 45253 | c.611A > G | p.E204G | Loss of Function | Missense_Mutation |
| NM_000546 | 10856 | c.880G > T | p.E294* | Loss of Function | Nonsense_Mutation |
| NM_000546 | 43990 | c.610G > A | p.E204K | Loss of Function | Missense_Mutation |
| NM_000546 | 45516 | c.662A > G | p.E221G | Loss of Function | Missense_Mutation |
| NM_000546 | 44207 | c.874delA | p.E294fs*51 | Loss of Function | Frame_Shift_Del |
| NM_000546 | 45670 | c.877delG | p.E294fs*51 | Loss of Function | Frame_Shift_Del |
| NM_000546 | 6621 | c.880delG | p.E294fs*51 | Loss of Function | Frame_Shift_Del |
| NM_000546 | 44853 | c.661G > A | p.E221K | Loss of Function | Missense_Mutation |
| NM_000546 | 44441 | c.813G > C | p.E271D | Loss of Function | Missense_Mutation |
| NM_000546 | 45284 | c.813G > A | p.E271E | Loss of Function | Synonymous_Mutation |
| NM_000546 | 10710 | c.892G > T | p.E298* | Loss of Function | Nonsense_Mutation |
| NM_000546 | 43879 | c.812A > G | p.E271G | Loss of Function | Missense_Mutation |
| NM_000546 | 11291 | c.1006G > T | p.E336* | Loss of Function | Nonsense_Mutation |
| NM_000546 | 11286 | c.1015G > T | p.E339* | Loss of Function | Nonsense_Mutation |
| NM_000546 | 11078 | c.1027G > T | p.E343* | Loss of Function | Nonsense_Mutation |
| NM_000546 | 10770 | c.1045G > T | p.E349* | Loss of Function | Nonsense_Mutation |
| NM_000546 | 43706 | c.811G > C | p.E271Q | Loss of Function | Missense_Mutation |
| NM_000546 | 43614 | c.854A > C | p.E285A | Loss of Function | Missense_Mutation |
| NM_000546 | 45649 | c.854A > G | p.E285G | Loss of Function | Missense_Mutation |
| NM_000546 | 44654 | c.400T > C | p.F134L | Loss of Function | Missense_Mutation |
| ENST00000269305 | 137087 | c.853G > A | p.E285K | Loss of Function | Missense_Mutation |
| NM_000546 | 43941 | c.400T > G | p.F134V | Loss of Function | Missense_Mutation |
| NM_000546 | 44162 | c.635_636delTT | p.F212fs*3 | Loss of Function | Frame_Shift_Del |
| NM_000546 | 44695 | c.634_635delTT | p.F212fs*3 | Loss of Function | Frame_Shift_Del |
| NM_000546 | 45138 | c.853G > C | p.E285Q | Loss of Function | Missense_Mutation |
| NM_000546 | 44227 | c.854A > T | p.E285V | Loss of Function | Missense_Mutation |
| ENST00000269305 | 99924 | c.856G > A | p.E286K | Loss of Function | Missense_Mutation |
| NM_000546 | 43621 | c.809T > G | p.F270C | Loss of Function | Missense_Mutation |
| NM_000546 | 43809 | c.808T > A | p.F270I | Loss of Function | Missense_Mutation |
| NM_000546 | 44156 | c.810T > A | p.F270L | Loss of Function | Missense_Mutation |
| NM_000546 | 44262 | c.808T > C | p.F270L | Loss of Function | Missense_Mutation |
| NM_000546 | 45297 | c.810T > G | p.F270L | Loss of Function | Missense_Mutation |
| NM_000546 | 11305 | c.809T > C | p.F270S | Loss of Function | Missense_Mutation |
| NM_000546 | 44737 | c.860A > G | p.E287G | Loss of Function | Missense_Mutation |
| NM_000546 | 44225 | c.859G > A | p.E287K | Loss of Function | Missense_Mutation |
| NM_000546 | 42813 | c.313G > T | p.G105C | Loss of Function | Missense_Mutation |
| NM_000546 | 44481 | c.313G > T | p.G105C | Loss of Function | Missense_Mutation |
| NM_000546 | 45801 | c.312delG | p.G105fs*18 | Loss of Function | Frame_Shift_Del |
| NM_000546 | 45179 | c.313G > C | p.G105R | Loss of Function | Missense_Mutation |
| NM_000546 | 45534 | c.882G > T | p.E294D | Loss of Function | Missense_Mutation |
| NM_000546 | 44715 | c.460G > T | p.G154C | Loss of Function | Missense_Mutation |
| NM_000546 | 44412 | c.882G > A | p.E294E | Loss of Function | Synonymous_Mutation |
| NM_000546 | 44726 | c.460_466delGGCACCC | p.G154fs*14 | Loss of Function | Frame_Shift_Del |
| NM_000546 | 43666 | c.462C > T | p.G154G | Loss of Function | Synonymous_Mutation |
| NM_000546 | 44298 | c.462C > A | p.G154G | Loss of Function | Synonymous_Mutation |
| NM_000546 | 43746 | c.881A > G | p.E294G | Loss of Function | Missense_Mutation |
| NM_000546 | 44127 | c.880G > A | p.E294K | Loss of Function | Missense_Mutation |
| NM_000546 | 6815 | c.461G > T | p.G154V | Loss of Function | Missense_Mutation |
| NM_000546 | 45824 | c.880G > C | p.E294Q | Loss of Function | Missense_Mutation |
| NM_000546 | 45820 | c.893A > T | p.E298V | Loss of Function | Missense_Mutation |
| NM_000546 | 44026 | c.559G > A | p.G187S | Loss of Function | Splice_Site |
| NM_000546 | 45169 | c.326T > C | p.F109S | Loss of Function | Missense_Mutation |
| NM_000546 | 44537 | c.595G > T | p.G199* | Loss of Function | Nonsense_Mutation |
| NM_000546 | 43949 | c.401T > G | p.F134C | Loss of Function | Missense_Mutation |
| NM_000546 | 45051 | c.597A > G | p.G199G | Loss of Function | Synonymous_Mutation |
| NM_000546 | 11319 | c.402T > G | p.F134L | Loss of Function | Missense_Mutation |
| NM_000546 | 44140 | c.596G > T | p.G199V | Loss of Function | Missense_Mutation |
| NM_000546 | 44506 | c.401T > C | p.F134S | Loss of Function | Missense_Mutation |
| NM_000546 | 45703 | c.634T > A | p.F212I | Loss of Function | Missense_Mutation |
| NM_000546 | 45868 | c.678C > T | p.G226G | Loss of Function | Synonymous_Mutation |
| NM_000546 | 44846 | c.636T > A | p.F212L | Loss of Function | Missense_Mutation |
| NM_000546 | 46214 | c.635T > C | p.F212S | Loss of Function | Missense_Mutation |
| NM_000546 | 44956 | c.808T > G | p.F270V | Loss of Function | Missense_Mutation |
| NM_000546 | 11524 | c.730G > T | p.G244C | Loss of Function | Missense_Mutation |
| NM_000546 | 10883 | c.731G > A | p.G244D | Loss of Function | Missense_Mutation |
| NM_000546 | 44940 | c.730delG | p.G244fs*3 | Loss of Function | Frame_Shift_Del |
| NM_000546 | 43656 | c.732C > G | p.G244G | Loss of Function | Synonymous_Mutation |
| NM_000546 | 44513 | c.732C > A | p.G244G | Loss of Function | Synonymous_Mutation |
| NM_000546 | 44787 | c.732C > T | p.G244G | Loss of Function | Synonymous_Mutation |
| NM_000546 | 44221 | c.730G > C | p.G244R | Loss of Function | Missense_Mutation |
| NM_000546 | 10941 | c.730G > A | p.G244S | Loss of Function | Missense_Mutation |
| NM_000546 | 43918 | c.809T > A | p.F270Y | Loss of Function | Missense_Mutation |
| NM_000546 | 13119 | c.322_324delGGT | p.G108del | Loss of Function | In_Frame_Del |
| NM_000546 | 11081 | c.733G > T | p.G245C | Loss of Function | Missense_Mutation |
| NM_000546 | 39293 | c.734G > A | p.G245D | Loss of Function | Missense_Mutation |
| NM_000546 | 43606 | c.734G > A | p.G245D | Loss of Function | Missense_Mutation |
| NM_000546 | 44642 | c.733delG | p.G245fs*2 | Loss of Function | Frame_Shift_Del |
| NM_000546 | 44900 | c.735C > T | p.G245G | Loss of Function | Synonymous_Mutation |
| ENST00000545858 | 179807 | c.455G > A | p.G152D | Loss of Function | Missense_Mutation |
| NM_000546 | 10957 | c.733G > C | p.G245R | Loss of Function | Missense_Mutation |
| NM_000546 | 6932 | c.733G > A | p.G245S | Loss of Function | Missense_Mutation |
| NM_000546 | 11196 | c.734G > T | p.G245V | Loss of Function | Missense_Mutation |
| NM_000546 | 44891 | c.796G > T | p.G266* | Loss of Function | Nonsense_Mutation |
| ENST00000545858 | 121037 | c.454G > A | p.G152S | Loss of Function | Missense_Mutation |
| NM_000546 | 10867 | c.797G > A | p.G266E | Loss of Function | Missense_Mutation |
| NM_000546 | 10794 | c.796G > A | p.G266R | Loss of Function | Missense_Mutation |
| NM_000546 | 11205 | c.796G > C | p.G266R | Loss of Function | Missense_Mutation |
| NM_000546 | 10958 | c.797G > T | p.G266V | Loss of Function | Missense_Mutation |
| NM_000546 | 43714 | c.836G > A | p.G279E | Loss of Function | Missense_Mutation |
| NM_000546 | 44896 | c.835_838delGGGA | p.G279fs*65 | Loss of Function | Frame_Shift_Del |
| NM_000546 | 46284 | c.837G > A | p.G279G | Loss of Function | Synonymous_Mutation |
| NM_000546 | 44603 | c.835G > A | p.G279R | Loss of Function | Missense_Mutation |
| NM_000546 | 45622 | c.461G > A | p.G154D | Loss of Function | Missense_Mutation |
| NM_000546 | 45506 | c.460_461GG > AT | p.G154I | Loss of Function | Missense_Mutation |
| NM_000546 | 44128 | c.879G > A | p.G293G | Loss of Function | Synonymous_Mutation |
| NM_000546 | 44131 | c.879G > C | p.G293G | Loss of Function | Synonymous_Mutation |
| NM_000546 | 43692 | c.460G > A | p.G154S | Loss of Function | Missense_Mutation |
| NM_000546 | 45275 | c.559G > T | p.G187C | Loss of Function | Missense_Mutation |
| NM_000546 | 87513 | c.902_903insC | p.G302fs*4 | Loss of Function | Frame_Shift_Ins |
| NM_000546 | 45998 | c.906G > C | p.G302G | Loss of Function | Synonymous_Mutation |
| NM_000546 | 11514 | c.1001G > T | p.G334V | Loss of Function | Missense_Mutation |
| NM_000546 | 44023 | c.560G > A | p.G187D | Loss of Function | Missense_Mutation |
| NM_000546 | 45479 | c.504C > T | p.H168H | Loss of Function | Synonymous_Mutation |
| NM_000546 | 44801 | c.503A > T | p.H168L | Loss of Function | Missense_Mutation |
| NM_000546 | 44808 | c.503A > C | p.H168P | Loss of Function | Missense_Mutation |
| NM_000546 | 45240 | c.560G > T | p.G187V | Loss of Function | Missense_Mutation |
| NM_000546 | 43989 | c.596G > A | p.G199E | Loss of Function | Missense_Mutation |
| NM_000546 | 44901 | c.532C > G | p.H178D | Loss of Function | Missense_Mutation |
| NM_000546 | 43978 | c.529delC | p.H178fs*69 | Loss of Function | Frame_Shift_Del |
| NM_000546 | 44134 | c.528delC | p.H178fs*69 | Loss of Function | Frame_Shift_Del |
| NM_000546 | 44659 | c.532delC | p.H178fs*69 | Loss of Function | Frame_Shift_Del |
| NM_000546 | 44971 | c.534C > T | p.H178H | Loss of Function | Synonymous_Mutation |
| NM_000546 | 43749 | c.595G > A | p.G199R | Loss of Function | Missense_Mutation |
| NM_000546 | 45739 | c.677G > C | p.G226A | Loss of Function | Missense_Mutation |
| NM_000546 | 11998 | c.534C > A | p.H178Q | Loss of Function | Missense_Mutation |
| NM_000546 | 46163 | c.534C > G | p.H178Q | Loss of Function | Missense_Mutation |
| NM_000546 | 44547 | c.677G > A | p.G226D | Loss of Function | Missense_Mutation |
| NM_000546 | 44776 | c.535C > G | p.H179D | Loss of Function | Missense_Mutation |
| NM_000546 | 44793 | c.537T > C | p.H179H | Loss of Function | Synonymous_Mutation |
| NM_000546 | 43635 | c.536A > T | p.H179L | Loss of Function | Missense_Mutation |
| NM_000546 | 44151 | c.535C > A | p.H179N | Loss of Function | Missense_Mutation |
| NM_000546 | 44218 | c.536A > C | p.H179P | Loss of Function | Missense_Mutation |
| NM_000546 | 11249 | c.537T > G | p.H179Q | Loss of Function | Missense_Mutation |
| NM_000546 | 44214 | c.537T > A | p.H179Q | Loss of Function | Missense_Mutation |
| NM_000546 | 10889 | c.536A > G | p.H179R | Loss of Function | Missense_Mutation |
| NM_000546 | 10768 | c.535C > T | p.H179Y | Loss of Function | Missense_Mutation |
| NM_000546 | 43584 | c.534_535CC > TT | p.H179Y | Loss of Function | Missense_Mutation |
| NM_000546 | 44002 | c.577C > G | p.H193D | Loss of Function | Missense_Mutation |
| NM_000546 | 44848 | c.579T > C | p.H193H | Loss of Function | Synonymous_Mutation |
| NM_000546 | 11066 | c.578A > T | p.H193L | Loss of Function | Missense_Mutation |
| NM_000546 | 45607 | c.676G > A | p.G226S | Loss of Function | Missense_Mutation |
| NM_000546 | 43833 | c.578A > C | p.H193P | Loss of Function | Missense_Mutation |
| NM_000546 | 10742 | c.578A > G | p.H193R | Loss of Function | Missense_Mutation |
| NM_000546 | 10672 | c.577C > T | p.H193Y | Loss of Function | Missense_Mutation |
| NM_000546 | 44399 | c.677G > T | p.G226V | Loss of Function | MissenseMutation |
| NM_000546 | 44372 | c.640delC | p.H214fs*33 | Loss of Function | Frame_Shift_Del |
| NM_000546 | 44638 | c.640_647delCATAGTGT | p.H214fs*5 | Loss of Function | Frame_Shift_Del |
| NM_000546 | 12013 | c.731G > C | p.G244A | Loss of Function | Missense_Mutation |
| NM_000546 | 42811 | c.641A > G | p.H214R | Loss of Function | Missense_Mutation |
| NM_000546 | 43687 | c.641A > G | p.H214R | Loss of Function | Missense_Mutation |
| NM_000546 | 43652 | c.731G > T | p.G244V | Loss of Function | Missense_Mutation |
| NM_000546 | 43965 | c.734G > C | p.G245A | Loss of Function | Missense_Mutation |
| ENST00000269305 | 179806 | c.734G > A | p.G245D | Loss of Function | Missense_Mutation |
| ENST00000413465 | 179805 | c.734G > A | p.G245D | Loss of Function | Missense_Mutation |
| NM_000546 | 45410 | c.733_734GG > AA | p.G245N | Loss of Function | Missense_Mutation |
| NM_000546 | 45069 | c.886delC | p.H296fs*49 | Loss of Function | Frame_Shift_Del |
| ENST00000269305 | 121035 | c.733G > A | p.G245S | Loss of Function | Missense_Mutation |
| ENST00000413465 | 121036 | c.733G > A | p.G245S | Loss of Function | Missense_Mutation |
| NM_000546 | 45488 | c.797G > C | p.G266A | Loss of Function | Missense_Mutation |
| ENST00000269305 | 99952 | c.797G > T | p.G266V | Loss of Function | Missense_Mutation |
| NM_000546 | 46032 | c.836G > T | p.G279V | Loss of Function | Missense_Mutation |
| NM_000546 | 43674 | c.835G > T | p.G279W | Loss of Function | Missense_Mutation |
| NM_000546 | 45417 | c.877G > A | p.G293R | Loss of Function | Missense_Mutation |
| NM_000546 | 43988 | c.905G > A | p.G302E | Loss of Function | Missense_Mutation |
| NM_000546 | 44830 | c.1066G > T | p.G356W | Loss of Function | Missense_Mutation |
| ENST00000545858 | 99918 | c.299A > T | p.H100L | Loss of Function | Missense_Mutation |
| NM_000546 | 43545 | c.503A > G | p.H168R | Loss of Function | Missense_Mutation |
| NM_000546 | 43861 | c.502C > T | p.H168Y | Loss of Function | Missense_Mutation |
| NM_000546 | 44633 | c.583A > T | p.I195F | Loss of Function | Missense_Mutation |
| NM_000546 | 44877 | c.584T > A | p.I195N | Loss of Function | Missense_Mutation |
| NM_000546 | 44539 | c.584T > G | p.I1955 | Loss of Function | Missense_Mutation |
| NM_000546 | 11089 | c.584T > C | p.I195T | Loss of Function | Missense_Mutation |
| NM_000546 | 43550 | c.694A > T | p.I232F | Loss of Function | Missense_Mutation |
| NM_000546 | 10715 | c.695T > A | p.I232N | Loss of Function | Missense_Mutation |
| NM_000546 | 45045 | c.695T > G | p.I232S | Loss of Function | Missense_Mutation |
| NM_000546 | 44601 | c.695T > C | p.I232T | Loss of Function | Missense_Mutation |
| NM_000546 | 44068 | c.532C > A | p.H178N | Loss of Function | Missense_Mutation |
| NM_000546 | 44457 | c.751_759delATCCTCACC | p.I251_T253delILT | Loss of Function | In_Frame_Del |
| NM_000546 | 44215 | c.533A > C | p.H178P | Loss of Function | Missense_Mutation |
| NM_000546 | 43967 | c.751A > T | p.I251F | Loss of Function | Missense_Mutation |
| NM_000546 | 44064 | c.748delC | p.I251fs*94 | Loss of Function | Frame_Shift_Del |
| NM_000546 | 44124 | c.751delA | p.I251fs*94 | Loss of Function | Frame_Shift_Del |
| NM_000546 | 44511 | c.753C > A | p.I251I | Loss of Function | Synonymous_Mutation |
| NM_000546 | 44120 | c.532C > T | p.H178Y | Loss of Function | Missense_Mutation |
| NM_000546 | 11374 | c.752T > A | p.I251N | Loss of Function | Missense_Mutation |
| NM_000546 | 43829 | c.752T > G | p.I251S | Loss of Function | Missense_Mutation |
| ENST00000269305 | 129848 | c.535C > T | p.H179Y | Loss of Function | Missense_Mutation |
| ENST00000413465 | 129849 | c.535C > T | p.H179Y | Loss of Function | Missense_Mutation |
| ENST00000269305 | 99919 | c.578A > T | p.H193L | Loss of Function | Missense_Mutation |
| ENST00000413465 | 99916 | c.578A > T | p.H193L | Loss of Function | Missense_Mutation |
| NM_000546 | 43935 | c.577C > A | p.H193N | Loss of Function | Missense_Mutation |
| NM_000546 | 45035 | c.761T > G | p.I254S | Loss of Function | Missense_Mutation |
| NM_000546 | 45115 | c.640C > G | p.H214D | Loss of Function | Missense_Mutation |
| NM_000546 | 44407 | c.642T > G | p.H214Q | Loss of Function | Missense_Mutation |
| NM_000546 | 43651 | c.763A > T | p.I255F | Loss of Function | Missense_Mutation |
| NM_000546 | 11244 | c.764T > A | p.I255N | Loss of Function | Missense_Mutation |
| NM_000546 | 10788 | c.764T > G | p.I255S | Loss of Function | Missense_Mutation |
| NM_000546 | 44112 | c.640C > T | p.H214Y | Loss of Function | Missense_Mutation |
| NM_000546 | 46031 | c.697C > G | p.H233D | Loss of Function | Missense_Mutation |
| NM_000546 | 45959 | c.698A > T | p.H233L | Loss of Function | Missense_Mutation |
| NM_000546 | 44641 | c.394A > T | p.K132* | Loss of Function | Nonsense_Mutation |
| NM_000546 | 10813 | c.394A > G | p.K132E | Loss of Function | Missense_Mutation |
| NM_000546 | 43661 | c.394delA | p.K132fs*38 | Loss of Function | Frame_Shift_Del |
| NM_000546 | 43592 | c.395A > T | p.K132M | Loss of Function | Missense_Mutation |
| NM_000546 | 10991 | c.396G > T | p.K132N | Loss of Function | Missense_Mutation |
| NM_000546 | 43963 | c.396G > C | p.K132N | Loss of Function | Missense_Mutation |
| NM_000546 | 44350 | c.699C > G | p.H233Q | Loss of Function | Missense_Mutation |
| NM_000546 | 11582 | c.395A > G | p.K132R | Loss of Function | Missense_Mutation |
| NM_000546 | 43912 | c.395A > C | p.K132T | Loss of Function | Missense_Mutation |
| NM_000546 | 10750 | c.490A > T | p.K164* | Loss of Function | Nonsense_Mutation |
| NM_000546 | 10762 | c.490A > G | p.K164E | Loss of Function | Missense_Mutation |
| NM_000546 | 45187 | c.490_499del10 | p.K164fs*3 | Loss of Function | Frame_Shift_Del |
| NM_000546 | 44861 | c.490delA | p.K164fs*6 | Loss of Function | Frame_Shift_Del |
| NM_000546 | 45103 | c.492G > A | p.K164K | Loss of Function | Synonymous_Mutation |
| NM_000546 | 44705 | c.697C > T | p.H233Y | Loss of Function | Missense_Mutation |
| NM_000546 | 44522 | c.887A > T | p.H296L | Loss of Function | Missense_Mutation |
| NM_000546 | 45306 | c.886C > A | p.H296N | Loss of Function | Missense_Mutation |
| NM_000546 | 43915 | c.886C > T | p.H296Y | Loss of Function | Missense_Mutation |
| NM_000546 | 44475 | c.871A > T | p.K291* | Loss of Function | Nonsense_Mutation |
| NM_000546 | 45494 | c.888_889CC > TT | p.H297Y | Loss of Function | Missense_Mutation |
| NM_000546 | 44897 | c.871_889del19 | p.K291fs*48 | Loss of Function | Frame_Shift_Del |
| NM_000546 | 46224 | c.873G > A | p.K291K | Loss of Function | Synonymous_Mutation |
| NM_000546 | 45803 | c.889C > T | p.H297Y | Loss of Function | Missense_Mutation |
| ENST00000414315 | 129851 | c.139C > T | p.H47Y | Loss of Function | Missense_Mutation |
| ENST00000414315 | 99917 | c.182A > T | p.H61L | Loss of Function | Missense_Mutation |
| ENST00000545858 | 129850 | c.256C > T | p.H86Y | Loss of Function | Missense_Mutation |
| NM_000546 | 44320 | c.484A > T | p.I162F | Loss of Function | Missense_Mutation |
| NM_000546 | 44694 | c.486C > A | p.I162I | Loss of Function | Missense_Mutation |
| NM_000546 | 45627 | c.486C > T | p.I162I | Loss of Function | Missense_Mutation |
| NM_000546 | 43773 | c.913A > T | p.K305* | Loss of Function | Nonsense_Mutation |
| NM_000546 | 44125 | c.486C > G | p.I162M | Loss of Function | Missense_Mutation |
| NM_000546 | 11966 | c.485T > A | p.I162N | Loss of Function | Missense_Mutation |
| NM_000546 | 11449 | c.388C > T | p.L130F | Loss of Function | Missense_Mutation |
| NM_000546 | 43898 | c.485T > G | p.I162S | Loss of Function | Missense_Mutation |
| NM_000546 | 45077 | c.390C > T | p.L130L | Loss of Function | Synonymous_Mutation |
| NM_000546 | 44413 | c.484A > G | p.I162V | Loss of Function | Missense_Mutation |
| NM_000546 | 11462 | c.388C > G | p.L130V | Loss of Function | Missense_Mutation |
| NM_000546 | 10995 | c.580C > T | p.L194F | Loss of Function | Missense_Mutation |
| NM_000546 | 43623 | c.581T > A | p.L194H | Loss of Function | Missense_Mutation |
| NM_000546 | 43929 | c.582T > C | p.L194L | Loss of Function | Synonymous_Mutation |
| NM_000546 | 43827 | c.581T > C | p.L194P | Loss of Function | Missense_Mutation |
| NM_000546 | 44571 | c.581T > G | p.L194R | Loss of Function | Missense_Mutation |
| NM_000546 | 44622 | c.694A > G | p.I232V | Loss of Function | Missense_Mutation |
| NM_000546 | 44650 | c.751_753delATC | p.I251del | Loss of Function | In_Frame_Del |
| NM_000546 | 44157 | c.601delT | p.L201fs*46 | Loss of Function | Frame_Shift_Del |
| NM_000546 | 43793 | c.617T > A | p.L206* | Loss of Function | Nonsense_Mutation |
| NM_000546 | 44852 | c.617delT | p.L206fs*41 | Loss of Function | Frame_Shift_Del |
| NM_000546 | 10931 | c.751A > C | p.I251L | Loss of Function | Missense_Mutation |
| NM_000546 | 11213 | c.752T > C | p.I251T | Loss of Function | Missense_Mutation |
| NM_000546 | 44541 | c.754delC | p.L252fs*93 | Loss of Function | Frame_Shift_Del |
| NM_000546 | 45407 | c.751A > G | p.I251V | Loss of Function | Missense_Mutation |
| NM_000546 | 44769 | c.755T > C | p.L252P | Loss of Function | Missense_Mutation |
| NM_000546 | 11929 | c.760_761AT > GA | p.I254D | Loss of Function | Missense_Mutation |
| NM_000546 | 11011 | c.794T > C | p.L265P | Loss of Function | Missense_Mutation |
| NM_000546 | 44092 | c.794T > G | p.L265R | Loss of Function | Missense_Mutation |
| NM_000546 | 45446 | c.865C > T | p.L289F | Loss of Function | Missense_Mutation |
| NM_000546 | 45688 | c.867C > T | p.L289L | Loss of Function | Synonymous_Mutation |
| NM_000546 | 45647 | c.760A > T | p.I254F | Loss of Function | Missense_Mutation |
| NM_000546 | 44535 | c.761T > A | p.I254N | Loss of Function | Missense_Mutation |
| NM_000546 | 46015 | c.1043T > A | p.L348* | Loss of Function | Nonsense_Mutation |
| NM_000546 | 46348 | c.1044G > T | p.L348F | Loss of Function | Missense_Mutation |
| NM_000546 | 45586 | c.397delA | p.M133fs*37 | Loss of Function | Frame_Shift_Del |
| NM_000546 | 44058 | c.761T > C | p.I254T | Loss of Function | Missense_Mutation |
| NM_000546 | 11781 | c.398T > A | p.M133K | Loss of Function | Missense_Mutation |
| NM_000546 | 44030 | c.760A > G | p.I254V | Loss of Function | Missense_Mutation |
| NM_000546 | 11181 | c.764T > C | p.I255T | Loss of Function | Missense_Mutation |
| NM_000546 | 44290 | c.763A > G | p.I255V | Loss of Function | Missense_Mutation |
| NM_000546 | 44986 | c.302A > G | p.K101R | Loss of Function | Missense_Mutation |
| NM_000546 | 11224 | c.394A > C | p.K132Q | Loss of Function | Missense_Mutation |
| NM_000546 | 44841 | c.491A > T | p.K164M | Loss of Function | Missense_Mutation |
| NM_000546 | 11369 | c.492G > T | p.K164N | Loss of Function | Missense_Mutation |
| NM_000546 | 44126 | c.507G > A | p.M169I | Loss of Function | Missense_Mutation |
| NM_000546 | 45490 | c.507G > T | p.M169I | Loss of Function | Missense_Mutation |
| NM_000546 | 44521 | c.490A > C | p.K164Q | Loss of Function | Missense_Mutation |
| NM_000546 | 44387 | c.491A > C | p.K164T | Loss of Function | Missense_Mutation |
| NM_000546 | 45162 | c.709delA | p.M237fs*10 | Loss of Function | Frame_Shift_Del |
| NM_000546 | 45862 | c.710delT | p.M237fs*10 | Loss of Function | Frame_Shift_Del |
| NM_000546 | 10834 | c.711G > A | p.M237I | Loss of Function | Missense_Mutation |
| NM_000546 | 11063 | c.711G > T | p.M237I | Loss of Function | Missense_Mutation |
| NM_000546 | 44415 | c.711G > C | p.M237I | Loss of Function | Missense_Mutation |
| NM_000546 | 43952 | c.710T > A | p.M237K | Loss of Function | Missense_Mutation |
| NM_000546 | 45050 | c.871A > G | p.K291E | Loss of Function | Missense_Mutation |
| NM_000546 | 44446 | c.873G > C | p.K291N | Loss of Function | Missense_Mutation |
| NM_000546 | 43747 | c.872A > G | p.K291R | Loss of Function | Missense_Mutation |
| NM_000546 | 44525 | c.709A > G | p.M237V | Loss of Function | Missense_Mutation |
| NM_000546 | 44433 | c.872A > C | p.K291T | Loss of Function | Missense_Mutation |
| NM_000546 | 44451 | c.874A > G | p.K292E | Loss of Function | Missense_Mutation |
| NM_000546 | 45611 | c.876A > C | p.K292N | Loss of Function | Missense_Mutation |
| NM_000546 | 43624 | c.875A > G | p.K292R | Loss of Function | Missense_Mutation |
| NM_000546 | 44346 | c.875A > C | p.K292T | Loss of Function | Missense_Mutation |
| NM_000546 | 44345 | c.915G > T | p.K305N | Loss of Function | Missense_Mutation |
| NM_000546 | 43743 | c.914A > G | p.K305R | Loss of Function | Missense_Mutation |
| NM_000546 | 46114 | c.389T > A | p.L130H | Loss of Function | Missense_Mutation |
| NM_000546 | 44664 | c.736_750del15 | p.M246_P250delMN | Loss of Function | In_Frame_Del |
| NM_000546 | 44903 | c.736delA | p.M246fs*1 | Loss of Function | Frame_Shift_Del |
| NM_000546 | 10757 | c.738G > C | p.M246I | Loss of Function | Missense_Mutation |
| NM_000546 | 44310 | c.738G > A | p.M246I | Loss of Function | Missense_Mutation |
| NM_000546 | 46136 | c.738G > T | p.M246I | Loss of Function | Missense_Mutation |
| NM_000546 | 44063 | c.389T > G | p.L130R | Loss of Function | Missense_Mutation |
| NM_000546 | 43777 | c.603G > C | p.L201F | Loss of Function | Missense_Mutation |
| NM_000546 | 11376 | c.737T > G | p.M246R | Loss of Function | Missense_Mutation |
| NM_000546 | 45489 | c.603G > T | p.L201F | Loss of Function | Missense_Mutation |
| NM_000546 | 43555 | c.736A > G | p.M246V | Loss of Function | Missense_Mutation |
| NM_000546 | 44247 | c.754_756delCTC | p.L252del | Loss of Function | In_Frame_Del |
| NM_000546 | 44054 | c.754C > T | p.L252F | Loss of Function | Missense_Mutation |
| NM_000546 | 45882 | c.391delA | p.N131fs*39 | Loss of Function | Frame_Shift_Del |
| NM_000546 | 45091 | c.755T > A | p.L252H | Loss of Function | Missense_Mutation |
| NM_000546 | 45393 | c.793C > A | p.L265M | Loss of Function | Missense_Mutation |
| NM_000546 | 43968 | c.866T > C | p.L289P | Loss of Function | Missense_Mutation |
| NM_000546 | 44070 | c.1031T > C | p.L344P | Loss of Function | Missense_Mutation |
| NM_000546 | 43859 | c.598delA | p.N200fs*47 | Loss of Function | Frame_Shift_Del |
| NM_000546 | 44206 | c.399G > T | p.M133I | Loss of Function | Missense_Mutation |
| NM_000546 | 43730 | c.398T > G | p.M133R | Loss of Function | Missense_Mutation |
| NM_000546 | 43641 | c.628delA | p.N210fs*37 | Loss of Function | Frame_Shift_Del |
| NM_000546 | 43723 | c.398T > C | p.M133T | Loss of Function | Missense_Mutation |
| ENST00000545858 | 99647 | c.432G > A | p.M144I | Loss of Function | Missense_Mutation |
| NM_000546 | 43891 | c.480G > A | p.M160I | Loss of Function | Missense_Mutation |
| NM_000546 | 45674 | c.480G > T | p.M160I | Loss of Function | Missense_Mutation |
| NM_000546 | 44305 | c.479T > A | p.M160K | Loss of Function | Missense_Mutation |
| NM_000546 | 44842 | c.478A > C | p.M160L | Loss of Function | Missense_Mutation |
| NM_000546 | 44328 | c.478A > G | p.M160V | Loss of Function | Missense_Mutation |
| NM_000546 | 45134 | c.715_726del12 | p.N239_C242delNSS | Loss of Function | In_Frame_Del |
| NM_000546 | 43851 | c.506T > C | p.M169T | Loss of Function | Missense_Mutation |
| NM_000546 | 10777 | c.715A > G | p.N239D | Loss of Function | Missense_Mutation |
| NM_000546 | 69195 | c.714_715insT | p.N239fs*1 | Loss of Function | Frame_Shift_Ins |
| NM_000546 | 44183 | c.715delA | p.N239fs*8 | Loss of Function | Frame_Shift_Del |
| NM_000546 | 44431 | c.505A > G | p.M169V | Loss of Function | Missense_Mutation |
| ENST00000269305 | 99648 | c.711G > A | p.M237I | Loss of Function | Missense_Mutation |
| NM_000546 | 44094 | c.716A > G | p.N239S | Loss of Function | Missense_Mutation |
| ENST00000413465 | 99646 | c.711G > A | p.M237I | Loss of Function | Missense_Mutation |
| NM_000546 | 44965 | c.709A > T | p.M237L | Loss of Function | Missense_Mutation |
| NM_000546 | 6546 | c.741_742CC > AT | p.N247_R248 > KW | Loss of Function | In_Frame_Del |
| NM_000546 | 45032 | c.710T > G | p.M237R | Loss of Function | Missense_Mutation |
| NM_000546 | 43995 | c.740A > T | p.N247I | Loss of Function | Missense_Mutation |
| NM_000546 | 45329 | c.710T > C | p.M237T | Loss of Function | Missense_Mutation |
| NM_000546 | 44428 | c.741C > T | p.N247N | Loss of Function | Synonymous_Mutation |
| NM_000546 | 44129 | c.729G > A | p.M243I | Loss of Function | Missense_Mutation |
| NM_000546 | 46228 | c.729G > C | p.M243I | Loss of Function | Missense_Mutation |
| NM_000546 | 44322 | c.728T > A | p.M243K | Loss of Function | Missense_Mutation |
| NM_000546 | 43726 | c.727A > T | p.M243L | Loss of Function | Missense_Mutation |
| NM_000546 | 43765 | c.727A > C | p.M243L | Loss of Function | Missense_Mutation |
| NM_000546 | 46208 | c.859_872del14 | p.N288fs*13 | Loss of Function | Frame_Shift_Del |
| NM_000546 | 45459 | c.862delA | p.N288fs*57 | Loss of Function | Frame_Shift_Del |
| NM_000546 | 44514 | c.728T > G | p.M243R | Loss of Function | Missense_Mutation |
| NM_000546 | 44536 | c.728T > C | p.M243T | Loss of Function | Missense_Mutation |
| NM_000546 | 44879 | c.1033delA | p.N345fs*25 | Loss of Function | Frame_Shift_Del |
| NM_000546 | 44396 | c.382delC | p.P128fs*42 | Loss of Function | Frame_Shift_Del |
| NM_000546 | 46131 | c.380delC | p.P128fs*42 | Loss of Function | Frame_Shift_Del |
| NM_000546 | 44844 | c.727A > G | p.M243V | Loss of Function | Missense_Mutation |
| NM_000546 | 44103 | c.737T > A | p.M246K | Loss of Function | Missense_Mutation |
| NM_000546 | 44749 | c.453C > T | p.P151P | Loss of Function | Synonymous_Mutation |
| NM_000546 | 45594 | c.453C > G | p.P151P | Loss of Function | Synonymous_Mutation |
| NM_000546 | 45992 | c.736A > T | p.M246L | Loss of Function | Missense_Mutation |
| NM_000546 | 10790 | c.455C > T | p.P152L | Loss of Function | Missense_Mutation |
| NM_000546 | 44061 | c.456G > A | p.P152P | Loss of Function | Synonymous_Mutation |
| NM_000546 | 44613 | c.455C > A | p.P152Q | Loss of Function | Missense_Mutation |
| NM_000546 | 45505 | c.455C > G | p.P152R | Loss of Function | Missense_Mutation |
| NM_000546 | 43582 | c.454C > T | p.P152S | Loss of Function | Missense_Mutation |
| NM_000546 | 11355 | c.737T > C | p.M246T | Loss of Function | Missense_Mutation |
| NM_000546 | 44212 | c.391_393delAAC | p.N131del | Loss of Function | In_Frame_Del |
| NM_000546 | 43964 | c.459C > T | p.P153P | Loss of Function | Synonymous_Mutation |
| NM_000546 | 44416 | c.459C > A | p.P153P | Loss of Function | Synonymous_Mutation |
| NM_000546 | 44589 | c.393_395delCAA | p.N131del | Loss of Function | In_Frame_Del |
| NM_000546 | 43535 | c.391A > C | p.N131H | Loss of Function | Missense_Mutation |
| NM_000546 | 43570 | c.529_546del18 | p.P177_C182delPHH | Loss of Function | In_Frame_Del |
| NM_000546 | 44730 | c.529_545del17 | p.P177fs*3 | Loss of Function | Frame_Shift_Del |
| NM_000546 | 44794 | c.392A > T | p.N131I | Loss of Function | Missense_Mutation |
| NM_000546 | 44097 | c.530C > T | p.P177L | Loss of Function | Missense_Mutation |
| NM_000546 | 43679 | c.531C > T | p.P177P | Loss of Function | Synonymous_Mutation |
| NM_000546 | 44818 | c.531C > G | p.P177P | Loss of Function | Synonymous_Mutation |
| NM_000546 | 10651 | c.530C > G | p.P177R | Loss of Function | Missense_Mutation |
| NM_000546 | 44474 | c.392A > G | p.N131S | Loss of Function | Missense_Mutation |
| NM_000546 | 43533 | c.391A > T | p.N131Y | Loss of Function | Missense_Mutation |
| NM_000546 | 46107 | c.599A > T | p.N200I | Loss of Function | Missense_Mutation |
| NM_000546 | 39455 | c.569delC | p.P190fs*57 | Loss of Function | Frame_Shift_Del |
| NM_000546 | 45320 | c.569delC | p.P190fs*57 | Loss of Function | Frame_Shift_Del |
| NM_000546 | 43657 | c.569C > T | p.P190L | Loss of Function | Missense_Mutation |
| NM_000546 | 44502 | c.599A > G | p.N200S | Loss of Function | Missense_Mutation |
| NM_000546 | 45441 | c.629A > G | p.N210S | Loss of Function | Missense_Mutation |
| NM_000546 | 11542 | c.703A > G | p.N235D | Loss of Function | Missense_Mutation |
| NM_000546 | 44784 | c.703_705delAAC | p.N235del | Loss of Function | In_Frame_Del |
| NM_000546 | 43860 | c.704A > T | p.N235I | Loss of Function | Missense_Mutation |
| NM_000546 | 45341 | c.571delC | p.P191fs*56 | Loss of Function | Frame_Shift_Del |
| NM_000546 | 43616 | c.704A > G | p.N235S | Loss of Function | Missense_Mutation |
| NM_000546 | 45620 | c.704A > C | p.N235T | Loss of Function | Missense_Mutation |
| NM_000546 | 45172 | c.703A > T | p.N235Y | Loss of Function | Missense_Mutation |
| NM_000546 | 45055 | c.715_720delAACAGT | p.N239_S240delNS | Loss of Function | In_Frame_Del |
| NM_000546 | 44689 | c.657C > T | p.P219P | Loss of Function | Synonymous_Mutation |
| NM_000546 | 44510 | c.717C > G | p.N239K | Loss of Function | Missense_Mutation |
| NM_000546 | 44647 | c.717C > A | p.N239K | Loss of Function | Missense_Mutation |
| NM_000546 | 43801 | c.716A > C | p.N239T | Loss of Function | Missense_Mutation |
| NM_000546 | 45870 | c.715A > T | p.N239Y | Loss of Function | Missense_Mutation |
| NM_000546 | 45005 | c.739A > G | p.N247D | Loss of Function | Missense_Mutation |
| NM_000546 | 45632 | c.741C > A | p.N247K | Loss of Function | Missense_Mutation |
| NM_000546 | 10771 | c.749C > T | p.P250L | Loss of Function | Missense_Mutation |
| NM_000546 | 44512 | c.740A > G | p.N247S | Loss of Function | Missense_Mutation |
| NM_000546 | 43588 | c.740A > C | p.N247T | Loss of Function | Missense_Mutation |
| NM_000546 | 43864 | c.739A > T | p.N247Y | Loss of Function | Missense_Mutation |
| NM_000546 | 43979 | c.802A > C | p.N268H | Loss of Function | Missense_Mutation |
| NM_000546 | 10814 | c.832C > G | p.P278A | Loss of Function | Missense_Mutation |
| NM_000546 | 44868 | c.804C > T | p.N268N | Loss of Function | Synonymous_Mutation |
| NM_000546 | 44871 | c.833delC | p.P278fs*67 | Loss of Function | Frame_Shift_Del |
| NM_000546 | 45178 | c.832delC | p.P278fs*67 | Loss of Function | Frame_Shift_Del |
| NM_000546 | 43755 | c.833C > A | p.P278H | Loss of Function | Missense_Mutation |
| NM_000546 | 10863 | c.833C > T | p.P278L | Loss of Function | Missense_Mutation |
| NM_000546 | 10887 | c.833C > G | p.P278R | Loss of Function | Missense_Mutation |
| NM_000546 | 10939 | c.832C > T | p.P278S | Loss of Function | Missense_Mutation |
| NM_000546 | 43697 | c.832C > A | p.P278T | Loss of Function | Missense_Mutation |
| NM_000546 | 44523 | c.863A > G | p.N288S | Loss of Function | Missense_Mutation |
| NM_000546 | 45332 | c.885T > C | p.P295P | Loss of Function | Synonymous_Mutation |
| NM_000546 | 43725 | c.862A > T | p.N288Y | Loss of Function | Missense_Mutation |
| NM_000546 | 45131 | c.383C > T | p.P128L | Loss of Function | Missense_Mutation |
| NM_000546 | 44397 | c.382C > T | p.P128S | Loss of Function | Missense_Mutation |
| NM_000546 | 44788 | c.454C > G | p.P152A | Loss of Function | Missense_Mutation |
| NM_000546 | 45184 | c.902delC | p.P301fs*44 | Loss of Function | Frame_Shift_Del |
| NM_000546 | 45487 | c.898delC | p.P301fs*44 | Loss of Function | Frame_Shift_Del |
| NM_000546 | 45546 | c.901delC | p.P301fs*44 | Loss of Function | Frame_Shift_Del |
| NM_000546 | 44165 | c.903A > G | p.P301P | Loss of Function | Synonymous_Mutation |
| ENST00000269305 | 129856 | c.455C > T | p.P152L | Loss of Function | Missense_Mutation |
| ENST00000413465 | 129857 | c.455C > T | p.P152L | Loss of Function | Missense_Mutation |
| NM_000546 | 44561 | c.454C > A | p.P152T | Loss of Function | Missense_Mutation |
| NM_000546 | 44367 | c.458C > T | p.P153L | Loss of Function | Missense_Mutation |
| NM_000546 | 43675 | c.457C > T | p.P153S | Loss of Function | Missense_Mutation |
| NM_000546 | 45660 | c.457C > A | p.P153T | Loss of Function | Missense_Mutation |
| NM_000546 | 45326 | c.530C > A | p.P177H | Loss of Function | Missense_Mutation |
| NM_000546 | 10650 | c.529C > T | p.P177S | Loss of Function | Missense_Mutation |
| NM_000546 | 44426 | c.568C > G | p.P190A | Loss of Function | Missense_Mutation |
| NM_000546 | 44032 | c.298C > T | p.Q100* | Loss of Function | Nonsense_Mutation |
| NM_000546 | 10886 | c.310C > T | p.Q104* | Loss of Function | Nonsense_Mutation |
| NM_000546 | 11166 | c.406C > T | p.Q136* | Loss of Function | Nonsense_Mutation |
| NM_000546 | 43767 | c.406C > G | p.Q136E | Loss of Function | Missense_Mutation |
| NM_000546 | 45089 | c.407A > C | p.Q136P | Loss of Function | Missense_Mutation |
| NM_000546 | 44665 | c.568_570delCCT | p.P190del | Loss of Function | In_Frame_Del |
| NM_000546 | 43632 | c.493C > T | p.Q165* | Loss of Function | Nonsense Mutation |
| NM_000546 | 44004 | c.569C > G | p.P190R | Loss of Function | Missense_Mutation |
| NM_000546 | 44682 | c.568C > T | p.P190S | Loss of Function | Missense_Mutation |
| NM_000546 | 44438 | c.568C > A | p.P190T | Loss of Function | Missense_Mutation |
| NM_000546 | 11333 | c.499C > T | p.Q167* | Loss of Function | Nonsense_Mutation |
| NM_000546 | 44275 | c.499_500delCA | p.Q167fs*13 | Loss of Function | Frame_Shift_Del |
| NM_000546 | 51646 | c.498 499insC | p.Q167fs*14 | Loss of Function | Frame_Shift_lns |
| NM_000546 | 44336 | c.499delC | p.Q167fs*3 | Loss of Function | Frame_Shift_Del |
| NM_000546 | 44234 | c.571_573delCCT | p.P191del | Loss of Function | In_Frame_Del |
| NM_000546 | 45140 | c.572_574delCTC | p.P191del | Loss of Function | In_Frame_Del |
| NM_000546 | 44299 | c.501G > A | p.Q167Q | Loss of Function | Synonymous_Mutation |
| ENST00000269305 | 111724 | c.572_574delCTC | p.P191delP | Loss of Function | In_Frame_Del |
| NM_000546 | 10733 | c.574C > T | p.Q192* | Loss of Function | Nonsense_Mutation |
| ENST00000413465 | 111721 | c.572_574delCTC | p.P191delP | Loss of Function | In_Frame_Del |
| NM_000546 | 43782 | c.576G > A | p.Q192Q | Loss of Function | Synonymous_Mutation |
| NM_000546 | 44351 | c.572C > T | p.P191L | Loss of Function | Missense_Mutation |
| NM_000546 | 44172 | c.572C > G | p.P191R | Loss of Function | Missense_Mutation |
| NM_000546 | 43682 | c.328C > T | p.R110C | Loss of Function | Missense_Mutation |
| NM_000546 | 43702 | c.571C > T | p.P191S | Loss of Function | Missense_Mutation |
| NM_000546 | 10716 | c.329G > T | p.R110L | Loss of Function | Missense_Mutation |
| NM_000546 | 11250 | c.329G > C | p.R110P | Loss of Function | Missense_Mutation |
| ENST00000414315 | 129859 | c.59C > T | p.P20L | Loss of Function | Missense_Mutation |
| NM_000546 | 46124 | c.466C > T | p.R156C | Loss of Function | Missense_Mutation |
| NM_000546 | 45896 | c.466delC | p.R156fs*14 | Loss of Function | Frame_Shift_Del |
| NM_000546 | 44439 | c.656C > T | p.P219L | Loss of Function | Missense_Mutation |
| NM_000546 | 43739 | c.467G > A | p.R156H | Loss of Function | Missense_Mutation |
| NM_000546 | 44076 | c.655C > T | p.P219S | Loss of Function | Missense_Mutation |
| NM_000546 | 10760 | c.467G > C | p.R156P | Loss of Function | Missense_Mutation |
| NM_000546 | 44301 | c.468C > G | p.R156R | Loss of Function | Synonymous_Mutation |
| NM_000546 | 44854 | c.655C > A | p.P219T | Loss of Function | Missense_Mutation |
| NM_000546 | 44921 | c.748_756delCCCATCCTC | p.P250_L252delPIL | Loss of Function | In_Frame_Del |
| NM_000546 | 43848 | c.472C > T | p.R158C | Loss of Function | Missense_Mutation |
| NM_000546 | 45019 | c.471_472CC > TT | p.R158C | Loss of Function | Missense_Mutation |
| NM_0000546 | 43831 | c.472_475delCGCG | p.R158fs*11 | Loss of Function | Frame_Shift_Del |
| NM_000546 | 43781 | c.472delC | p.R158fs*12 | Loss of Function | Frame_Shift_Del |
| NM_000546 | 11087 | c.472C > G | p.R158G | Loss of Function | Missense_Mutation |
| NM_000546 | 10690 | c.473G > A | p.R158H | Loss of Function | Missense_Mutation |
| NM_000546 | 10714 | c.473G > T | p.R158L | Loss of Function | Missense_Mutation |
| NM_000546 | 44096 | c.748C > G | p.P250A | Loss of Function | Missense_Mutation |
| NM_000546 | 43940 | c.474C > T | p.R158R | Loss of Function | Synonymous_Mutation |
| NM_000546 | 46393 | c.520_536del17 | p.R174fs*1 | Loss of Function | Frame_Shift_Del |
| NM_000546 | 44725 | c.522delG | p.R174fs*73 | Loss of Function | Frame_Shift_Del |
| NM_000546 | 44609 | c.748_749CC > TT | p.P250F | Loss of Function | Missense_Mutation |
| NM_000546 | 44476 | c.749C > A | p.P250H | Loss of Function | Missense_Mutation |
| NM_000546 | 45034 | c.748_749CC > AA | p.P250N | Loss of Function | Missense_Mutation |
| NM_000546 | 43957 | c.750C > T | p.P250P | Loss of Function | Synonymous_Mutation |
| NM_000546 | 44742 | c.523_540dell8 | p.R175_E180delRCP | Loss of Function | Frame_Shift_Del |
| NM_000546 | 43680 | c.523C > T | p.R175C | Loss of Function | Missense_Mutation |
| NM_000546 | 10870 | c.523C > G | p.R175G | Loss of Function | Missense_Mutation |
| NM_000546 | 10648 | c.524G > A | p.R175H | Loss of Function | Missense_Mutation |
| NM_000546 | 44464 | c.749_750CC > AG | p.P250Q | Loss of Function | Missense_Mutation |
| NM_000546 | 43695 | c.748C > T | p.P250S | Loss of Function | Missense_Mutation |
| NM_000546 | 44566 | c.525C > G | p.R175R | Loss of Function | Synonymous_Mutation |
| NM_000546 | 45515 | c.525C > T | p.R175R | Loss of Function | Synonymous_Mutation |
| ENST00000269305 | 99725 | c.832C > G | p.P278A | Loss of Function | Missense_Mutation |
| NM_000546 | 11090 | c.541C > T | p.R181C | Loss of Function | Missense_Mutation |
| NM_000546 | 43587 | c.832_833CC > TT | p.P278F | Loss of Function | Missense_Mutation |
| ENST00000269305 | 129831 | c.833C > T | p.P278L | Loss of Function | Missense_Mutation |
| NM_000546 | 45046 | c.542G > C | p.R181P | Loss of Function | Missense_Mutation |
| NM_000546 | 43728 | c.543C > T | p.R181R | Loss of Function | Synonymous_Mutation |
| NM_000546 | 10705 | c.586C > T | p.R196* | Loss of Function | Nonsense_Mutation |
| NM_000546 | 45021 | c.585_586CC > TT | p.R196* | Loss of Function | Nonsense_Mutation |
| NM_000546 | 44757 | c.586delC | p.R196fs*51 | Loss of Function | Frame_Shift_Del |
| NM_000546 | 43814 | c.587G > C | p.R196P | Loss of Function | Missense_Mutation |
| ENST00000269305 | 139044 | c.832C > T | p.P278S | Loss of Function | Missense_Mutation |
| NM_000546 | 44569 | c.588A > G | p.R196R | Loss of Function | Synonymous_Mutation |
| NM_000546 | 44615 | c.586C > A | p.R196R | Loss of Function | Synonymous_Mutation |
| NM_000546 | 45233 | c.884C > T | p.P295L | Loss of Function | Missense_Mutation |
| NM_000546 | 44750 | c.883C > T | p.P295S | Loss of Function | Missense_Mutation |
| NM_000546 | 45311 | c.898C > G | p.P300A | Loss of Function | Missense_Mutation |
| NM_000546 | 43766 | c.899C > T | p.P300L | Loss of Function | Missense_Mutation |
| NM_000546 | 44729 | c.898C > T | p.P300S | Loss of Function | Missense_Mutation |
| NM_000546 | 44753 | c.901C > T | p.P301S | Loss of Function | Missense_Mutation |
| NM_000546 | 11290 | c.625A > T | p.R209* | Loss of Function | Nonsense Mutation |
| NM_000546 | 96575 | c.625_634del10 | p.R209fs*35 | Loss of Function | Frame_Shift_Del |
| NM_000546 | 45438 | c.626delG | p.R209fs*38 | Loss of Function | Frame_Shift_Del |
| NM_000546 | 13120 | c.626_627delGA | p.R209fs*6 | Loss of Function | Frame_Shift_Del |
| NM_000546 | 6482 | c.625_626delAG | p.R209fs*6 | Loss of Function | Frame_Shift_Del |
| ENST00000414315 | 111722 | c.176_178delCTC | p.P59delP | Loss of Function | In_Frame_Del |
| ENST00000545858 | 129858 | c.176C > T | p.P59L | Loss of Function | Missense_Mutation |
| NM_000546 | 10654 | c.637C > T | p.R213* | Loss of Function | Nonsense_Mutation |
| NM_000546 | 43807 | c.637delC | p.R213fs*34 | Loss of Function | Frame_Shift_Del |
| NM_000546 | 44358 | c.634delT | p.R213fs*34 | Loss of Function | Frame_Shift_Del |
| NM_000546 | 45777 | c.633delT | p.R213fs*34 | Loss of Function | Frame_Shift_Del |
| NM_000546 | 44102 | c.637C > G | p.R213G | Loss of Function | Missense_Mutation |
| NM_000546 | 43650 | c.638G > T | p.R213L | Loss of Function | Missense_Mutation |
| NM_000546 | 11860 | c.638G > C | p.R213P | Loss of Function | Missense_Mutation |
| NM_000546 | 10735 | c.638G > A | p.R213Q | Loss of Function | Missense_Mutation |
| NM_000546 | 45116 | c.742delC | p.R248fs*97 | Loss of Function | Frame_Shift_Del |
| NM_000546 | 11564 | c.742C > G | p.R248G | Loss of Function | Missense_Mutation |
| NM_000546 | 45543 | c.743_744GG > TT | p.R248L | Loss of Function | Missense_Mutation |
| NM_000546 | 6549 | c.743G > T | p.R248L | Loss of Function | Missense_Mutation |
| NM_000546 | 11491 | c.743G > C | p.R248P | Loss of Function | Missense_Mutation |
| NM_000546 | 10662 | c.743G > A | p.R248Q | Loss of Function | Missense_Mutation |
| NM_000546 | 44908 | c.743_744GG > AA | p.R248Q | Loss of Function | Missense_Mutation |
| NM_000546 | 46265 | c.224C > G | p.P75R | Loss of Function | Missense_Mutation |
| NM_000546 | 44287 | c.229C > G | p.P77A | Loss of Function | Missense_Mutation |
| NM_000546 | 43910 | c.245C > T | p.P82L | Loss of Function | Missense_Mutation |
| NM_000546 | 10656 | c.742C > T | p.R248W | Loss of Function | Missense_Mutation |
| NM_000546 | 6545 | c.741_742CC > TT | p.R248W | Loss of Function | Missense_Mutation |
| NM_000546 | 44916 | c.746delG | p.R249fs*96 | Loss of Function | Frame_Shift_Del |
| NM_000546 | 10668 | c.745A > G | p.R249G | Loss of Function | Missense_Mutation |
| NM_000546 | 44091 | c.746G > A | p.R249K | Loss of Function | Missense_Mutation |
| NM_000546 | 43871 | c.746G > T | p.R249M | Loss of Function | Missense_Mutation |
| NM_000546 | 45918 | c.253C > T | p.P85S | Loss of Function | Missense_Mutation |
| NM_000546 | 10785 | c.747G > C | p.R249S | Loss of Function | Missense_Mutation |
| NM_000546 | 10817 | c.747G > T | p.R249S | Loss of Function | Missense_Mutation |
| NM_000546 | 43665 | c.746G > C | p.R249T | Loss of Function | Missense_Mutation |
| NM_000546 | 43629 | c.745A > T | p.R249W | Loss of Function | Missense_Mutation |
| NM_000546 | 11392 | c.800G > C | p.R267P | Loss of Function | Missense_Mutation |
| NM_000546 | 43923 | c.800G > A | p.R267Q | Loss of Function | Missense_Mutation |
| NM_000546 | 43544 | c.260C > A | p.P87Q | Loss of Function | Missense_Mutation |
| NM_000546 | 11183 | c.799C > T | p.R267W | Loss of Function | Missense_Mutation |
| NM_000546 | 10659 | c.817C > T | p.R273C | Loss of Function | Missense_Mutation |
| NM_000546 | 44701 | c.817delC | p.R273fs*72 | Loss of Function | Frame_Shift_Del |
| NM_000546 | 43688 | c.265C > T | p.P89S | Loss of Function | Missense_Mutation |
| NM_000546 | 10660 | c.818G > A | p.R273H | Loss of Function | Missense_Mutation |
| NM_000546 | 10779 | c.818G > T | p.R273L | Loss of Function | Missense_Mutation |
| NM_000546 | 43896 | c.818G > C | p.R273P | Loss of Function | Missense_Mutation |
| NM_000546 | 43909 | c.817C > A | p.R273S | Loss of Function | Missense_Mutation |
| NM_000546 | 44390 | c.838A > T | p.R280* | Loss of Function | Nonsense_Mutation |
| ENST00000545858 | 111723 | c.293_295delCTC | p.P98delP | Loss of Function | In_Frame_Del |
| NM_000546 | 44005 | c.835delG | p.R280fs*65 | Loss of Function | Frame_Shift_Del |
| NM_000546 | 11123 | c.838A > G | p.R280G | Loss of Function | Missense_Mutation |
| NM_000546 | 11287 | c.839G > T | p.R280I | Loss of Function | Missense_Mutation |
| NM_000546 | 10728 | c.839G > A | p.R280K | Loss of Function | Missense_Mutation |
| NM_000546 | 44568 | c.840A > G | p.R280R | Loss of Function | Synonymous_Mutation |
| NM_000546 | 44171 | c.840A > T | p.R280S | Loss of Function | Missense_Mutation |
| NM_000546 | 44233 | c.840A > C | p.R280S | Loss of Function | Missense_Mutation |
| NM_000546 | 10724 | c.839G > C | p.R280T | Loss of Function | Missense_Mutation |
| NM_000546 | 10992 | c.844C > G | p.R282G | Loss of Function | Missense_Mutation |
| NM_000546 | 44681 | c.293C > T | p.P98L | Loss of Function | Missense_Mutation |
| NM_000546 | 12296 | c.292C > T | p.P98S | Loss of Function | Missense_Mutation |
| NM_000546 | 44338 | c.845G > A | p.R282Q | Loss of Function | Missense_Mutation |
| NM_000546 | 44724 | c.846G > A | p.R282R | Loss of Function | Synonymous_Mutation |
| NM_000546 | 44918 | c.844C > A | p.R282R | Loss of Function | Synonymous_Mutation |
| NM_000546 | 10704 | c.844C > T | p.R282W | Loss of Function | Missense_Mutation |
| NM_000546 | 43585 | c.843_844CC > TT | p.R282W | Loss of Function | Missense_Mutation |
| NM_000546 | 45293 | c.407A > G | p.Q136R | Loss of Function | Missense_Mutation |
| NM_000546 | 45891 | c.847_866del20 | p.R283fs*16 | Loss of Function | Frame_Shift_Del |
| NM_000546 | 45188 | c.847delC | p.R283fs*62 | Loss of Function | Frame_Shift_Del |
| NM_000546 | 44850 | c.494A > T | p.Q165L | Loss of Function | Missense_Mutation |
| NM_000546 | 44851 | c.494A > C | p.Q165P | Loss of Function | Missense_Mutation |
| NM_000546 | 44308 | c.494A > G | p.Q165R | Loss of Function | Missense_Mutation |
| NM_000546 | 10743 | c.848G > C | p.R283P | Loss of Function | Missense_Mutation |
| NM_000546 | 43977 | c.849C > T | p.R283R | Loss of Function | Synonymous_Mutation |
| NM_000546 | 45679 | c.868C > T | p.R290C | Loss of Function | Missense_Mutation |
| NM_000546 | 45626 | c.501G > T | p.Q167H | Loss of Function | Missense_Mutation |
| NM_000546 | 45342 | c.500A > T | p.Q167L | Loss of Function | Missense_Mutation |
| NM_000546 | 10663 | c.916C > T | p.R306* | Loss of Function | Nonsense_Mutation |
| NM_000546 | 11071 | c.1009C > T | p.R337C | Loss of Function | Missense_Mutation |
| NM_000546 | 43882 | c.1010G > A | p.R337H | Loss of Function | Missense_Mutation |
| NM_000546 | 11411 | c.1010G > T | p.R337L | Loss of Function | Missense_Mutation |
| NM_000546 | 11073 | c.1024C > T | p.R342* | Loss of Function | Nonsense Mutation |
| NM_000546 | 18597 | c.1024delC | p.R342fs*3 | Loss of Function | Frame_Shift_Del |
| NM_000546 | 43795 | c.1023delC | p.R342fs*3 | Loss of Function | Frame_Shift_Del |
| NM_000546 | 45639 | c.1024delC | p.R342fs*3 | Loss of Function | Frame_Shift_Del |
| NM_000546 | 45276 | c.1025G > C | p.R342P | Loss of Function | Missense_Mutation |
| NM_000546 | 43709 | c.500A > G | p.Q167R | Loss of Function | Missense_Mutation |
| NM_000546 | 45044 | c.576G > T | p.Q192H | Loss of Function | Missense_Mutation |
| NM_000546 | 44849 | c.575A > G | p.Q192R | Loss of Function | Missense_Mutation |
| NM_000546 | 45944 | c.318C > G | p.S106R | Loss of Function | Missense_Mutation |
| NM_000546 | 40942 | c.380C > T | p.S127F | Loss of Function | Missense_Mutation |
| NM_000546 | 45536 | c.1061A > G | p.Q354R | Loss of Function | Missense_Mutation |
| NM_000546 | 46115 | c.329G > A | p.R110H | Loss of Function | Missense_Mutation |
| NM_000546 | 44687 | c.379T > C | p.S127P | Loss of Function | Missense_Mutation |
| ENST00000269305 | 99929 | c.329G > T | p.R110L | Loss of Function | Missense_Mutation |
| NM_000546 | 43970 | c.380C > A | p.S127Y | Loss of Function | Missense_Mutation |
| NM_000546 | 11508 | c.497C > A | p.S166* | Loss of Function | Nonsense_Mutation |
| NM_000546 | 44467 | c.497C > G | p.S166* | Loss of Function | Nonsense_Mutation |
| ENST00000413465 | 99928 | c.329G > T | p.R110L | Loss of Function | Missense_Mutation |
| ENST00000545858 | 242000 | c.359G > T | p.R120L | Loss of Function | Missense_Mutation |
| ENST00000545858 | 99021 | c.464G > A | p.R155Q | Loss of Function | Missense_Mutation |
| NM_000546 | 10706 | c.548C > G | p.S183* | Loss of Function | Nonsense_Mutation |
| NM_000546 | 11717 | c.548C > A | p.S183* | Loss of Function | Nonsense_Mutation |
| ENST00000545858 | 120006 | c.463C > T | p.R155W | Loss of Function | Missense_Mutation |
| NM_000546 | 46001 | c.466_486del21 | p.R156_I162delRVR | Loss of Function | In_Frame_Del |
| NM_000546 | 45314 | c.553delA | p.S185fs*62 | Loss of Function | Frame_Shift_Del |
| NM_000546 | 45154 | c.466C > G | p.R156G | Loss of Function | Missense_Mutation |
| NM_000546 | 43548 | c.467G > T | p.R156L | Loss of Function | Missense_Mutation |
| NM_000546 | 43744 | c.466C > A | p.R156S | Loss of Function | Missense_Mutation |
| NM_000546 | 44267 | c.472_477delCGCGCC | p.R158_A159delRA | Loss of Function | In_Frame_Del |
| NM_000546 | 44887 | c.644delG | p.S215fs*32 | Loss of Function | Frame_Shift_Del |
| NM_000546 | 43951 | c.643A > G | p.S215G | Loss of Function | Missense_Mutation |
| NM_000546 | 11450 | c.644G > T | p.S215I | Loss of Function | Missense_Mutation |
| ENST00000269305 | 220779 | c.473G > A | p.R158H | Loss of Function | Missense_Mutation |
| NM_000546 | 44979 | c.645T > G | p.S215R | Loss of Function | Missense_Mutation |
| NM_000546 | 45122 | c.645T > A | p.S215R | Loss of Function | Missense_Mutation |
| NM_000546 | 46000 | c.643A > C | p.S215R | Loss of Function | Missense_Mutation |
| ENST00000413465 | 220778 | c.473G > A | p.R158H | Loss of Function | Missense_Mutation |
| NM_000546 | 43615 | c.473G > C | p.R158P | Loss of Function | Missense_Mutation |
| NM_000546 | 44524 | c.521G > A | p.R174K | Loss of Function | Missense_Mutation |
| NM_000546 | 45671 | c.521G > T | p.R174M | Loss of Function | Missense_Mutation |
| NM_000546 | 44217 | c.718delA | p.S240fs*7 | Loss of Function | Frame_Shift_Del |
| NM_000546 | 43973 | c.718A > G | p.S240G | Loss of Function | Missense_Mutation |
| NM_000546 | 44518 | c.522G > A | p.R174R | Loss of Function | Synonymous_Mutation |
| NM_000546 | 44782 | c.520A > T | p.R174W | Loss of Function | Missense_Mutation |
| ENST00000269305 | 99914 | c.524G > A | p.R175H | Loss of Function | Missense_Mutation |
| NM_000546 | 44838 | c.720T > C | p.S240S | Loss of Function | Synonymous_Mutation |
| ENST00000413465 | 99022 | c.524G > A | p.R175H | Loss of Function | Missense_Mutation |
| NM_000546 | 10718 | c.524G > T | p.R175L | Loss of Function | Missense_Mutation |
| NM_000546 | 10709 | c.722C > G | p.S241C | Loss of Function | Missense_Mutation |
| NM_000546 | 45416 | c.524G > C | p.R175P | Loss of Function | Missense_Mutation |
| NM_000546 | 43931 | c.523C > A | p.R175S | Loss of Function | Missense_Mutation |
| NM_000546 | 10812 | c.722C > T | p.S241F | Loss of Function | Missense_Mutation |
| NM_000546 | 45017 | c.722_723CC > TT | p.S241F | Loss of Function | Missense_Mutation |
| NM_000546 | 43645 | c.721delT | p.S241fs*6 | Loss of Function | Frame_Shift_Del |
| NM_000546 | 44578 | c.721T > C | p.S241P | Loss of Function | Missense_Mutation |
| NM_000546 | 10738 | c.542G > A | p.R181H | Loss of Function | Missense_Mutation |
| NM_000546 | 10935 | c.722C > A | p.S241Y | Loss of Function | Missense_Mutation |
| NM_000546 | 44152 | c.542G > T | p.R181L | Loss of Function | Missense_Mutation |
| NM_000546 | 44599 | c.587G > A | p.R196Q | Loss of Function | Missense_Mutation |
| NM_000546 | 46074 | c.604C > T | p.R202C | Loss of Function | Missense_Mutation |
| NM_000546 | 43594 | c.605G > A | p.R202H | Loss of Function | Missense_Mutation |
| NM_000546 | 44925 | c.605G > T | p.R202L | Loss of Function | Missense_Mutation |
| NM_000546 | 43608 | c.605G > C | p.R202P | Loss of Function | Missense_Mutation |
| NM_000546 | 44074 | c.605_606GT > CG | p.R202P | Loss of Function | Missense_Mutation |
| NM_000546 | 44237 | c.904delG | p.S303fs*42 | Loss of Function | Frame_Shift_Del |
| NM_000546 | 44174 | c.604C > A | p.R202S | Loss of Function | Missense_Mutation |
| NM_000546 | 45995 | c.626G > A | p.R209K | Loss of Function | Missense_Mutation |
| NM_000546 | 45257 | c.626G > C | p.R209T | Loss of Function | Missense_Mutation |
| NM_000546 | 18610 | c.267delC | p.S90fs*33 | Loss of Function | Frame_Shift_Del |
| NM_000546 | 45500 | c.281C > A | p.S94* | Loss of Function | Nonsense Mutation |
| ENST00000269305 | 241998 | c.638G > T | p.R213L | Loss of Function | Missense_Mutation |
| ENST00000413465 | 241997 | c.638G > T | p.R213L | Loss of Function | Missense_Mutation |
| ENST00000269305 | 99602 | c.743G > A | p.R248Q | Loss of Function | Missense_Mutation |
| ENST00000413465 | 99020 | c.743G > A | p.R248Q | Loss of Function | Missense_Mutation |
| NM_000546 | 85574 | c.291_295delCCCTT | p.S99fs*48 | Loss of Function | Frame_Shift_Del |
| NM_000546 | 44257 | c.301delA | p.T102fs*21 | Loss of Function | Frame_Shift_Del |
| NM_000546 | 44920 | c.742C > A | p.R248R | Loss of Function | Synonymous_Mutation |
| NM_000546 | 44303 | c.463A > G | p.T155A | Loss of Function | Missense_Mutation |
| NM_000546 | 44009 | c.463_470delACCCGCGT | p.T155fs*23 | Loss of Function | Frame_Shift_Del |
| NM_000546 | 44033 | c.464C > T | p.T155I | Loss of Function | Missense_Mutation |
| NM_000546 | 11218 | c.464C > A | p.T155N | Loss of Function | Missense_Mutation |
| NM_000546 | 10912 | c.463A > C | p.T155P | Loss of Function | Missense_Mutation |
| NM_000546 | 43670 | c.465C > T | p.T155T | Loss of Function | Synonymous_Mutation |
| NM_000546 | 45084 | c.744G > A | p.R248R | Loss of Function | Synonymous_Mutation |
| NM_000546 | 44384 | c.510G > A | p.T170T | Loss of Function | Synonymous_Mutation |
| NM_000546 | 45541 | c.510G > T | p.T170T | Loss of Function | Synonymous_Mutation |
| NM_000546 | 45735 | c.744G > C | p.R248R | Loss of Function | Synonymous_Mutation |
| NM_000546 | 44371 | c.631delA | p.T211fs*36 | Loss of Function | Frame_Shift_Del |
| NM_000546 | 43939 | c.632C > T | p.T211I | Loss of Function | Missense_Mutation |
| ENST00000269305 | 120007 | c.742C > T | p.R248W | Loss of Function | Missense_Mutation |
| NM_000546 | 46211 | c.633T > C | p.T211T | Loss of Function | Synonymous_Mutation |
| NM_000546 | 45157 | c.688delA | p.T230fs*17 | Loss of Function | Frame_Shift_Del |
| NM_000546 | 44458 | c.688_698del11 | p.T230fs*6 | Loss of Function | Frame_Shift_Del |
| ENST00000413465 | 120005 | c.742C > T | p.R248W | Loss of Function | Missense_Mutation |
| NM_000546 | 44625 | c.747G > A | p.R249R | Loss of Function | Synonymous_Mutation |
| NM_000546 | 44271 | c.688A > C | p.T230P | Loss of Function | Missense_Mutation |
| ENST00000269305 | 131478 | c.747G > T | p.R249S | Loss of Function | Missense_Mutation |
| ENST00000413465 | 131479 | c.747G > T | p.R249S | Loss of Function | Missense_Mutation |
| NM_000546 | 45784 | c.691delA | p.T231fs*16 | Loss of Function | Frame_Shift_Del |
| NM_000546 | 45706 | c.801G > T | p.R267R | Loss of Function | Synonymous_Mutation |
| ENST00000269305 | 179804 | c.799C > T | p.R267W | Loss of Function | Missense_Mutation |
| NM_000546 | 44113 | c.693C > T | p.T231T | Loss of Function | Synonymous_Mutation |
| ENST00000414315 | 220780 | c.77G > A | p.R26H | Loss of Function | Missense_Mutation |
| NM_000546 | 44460 | c.757_760delACCA | p.T253fs*91 | Loss of Function | Frame_Shift_Del |
| ENST00000269305 | 99933 | c.817C > T | p.R273C | Loss of Function | Missense_Mutation |
| NM_000546 | 43843 | c.817C > G | p.R273G | Loss of Function | Missense_Mutation |
| ENST00000269305 | 99729 | c.818G > A | p.R273H | Loss of Function | Missense_Mutation |
| NM_000546 | 44843 | c.759C > T | p.T253T | Loss of Function | Synonymous_Mutation |
| NM_000546 | 45843 | c.838_843delAGAGAC | p.R280_D281delRD | Loss of Function | In_Frame_Del |
| ENST00000269305 | 129830 | c.839G > A | p.R280K | Loss of Function | Missense_Mutation |
| NM_000546 | 44470 | c.845G > T | p.R282L | Loss of Function | Missense_Mutation |
| NM_000546 | 44352 | c.850A > C | p.T284P | Loss of Function | Missense_Mutation |
| NM_000546 | 44835 | c.852A > T | p.T284T | Loss of Function | Synonymous_Mutation |
| NM_000546 | 44306 | c.845G > C | p.R282P | Loss of Function | Missense_Mutation |
| NM_000546 | 44417 | c.910delA | p.T304fs*41 | Loss of Function | Frame_Shift_Del |
| ENST00000269305 | 99925 | c.844C > T | p.R282W | Loss of Function | Missense_Mutation |
| NM_000546 | 10911 | c.847C > T | p.R283C | Loss of Function | Missense_Mutation |
| NM_000546 | 46035 | c.847C > G | p.R283G | Loss of Function | Missense_Mutation |
| NM_000546 | 11483 | c.848G > A | p.R283H | Loss of Function | Missense_Mutation |
| NM_000546 | 10670 | c.469G > T | p.V157F | Loss of Function | Missense_Mutation |
| NM_000546 | 43710 | c.468delC | p.V157fs*13 | Loss of Function | Frame_Shift_Del |
| NM_000546 | 45111 | c.469_473delGTCCG | p.V157fs*22 | Loss of Function | Frame_Shift_Del |
| NM_000546 | 43903 | c.470T > G | p.V157G | Loss of Function | Missense_Mutation |
| NM_000546 | 44463 | c.848G > T | p.R283L | Loss of Function | Missense_Mutation |
| NM_000546 | 44017 | c.869G > A | p.R290H | Loss of Function | Missense_Mutation |
| NM_000546 | 43934 | c.471C > A | p.V157V | Loss of Function | Synonymous_Mutation |
| NM_000546 | 44526 | c.471C > T | p.V157V | Loss of Function | Synonymous_Mutation |
| NM_000546 | 44639 | c.869G > T | p.R290L | Loss of Function | Missense_Mutation |
| NM_000546 | 45278 | c.1025G > A | p.R342Q | Loss of Function | Missense_Mutation |
| NM_000546 | 44240 | c.514G > T | p.V172F | Loss of Function | Missense_Mutation |
| NM_000546 | 45906 | c.514delG | p.V172fs*2 | Loss of Function | Frame_Shift_Del |
| NM_000546 | 45047 | c.515T > G | p.V172G | Loss of Function | Missense_Mutation |
| ENST00000414315 | 99023 | c.128G > A | p.R43H | Loss of Function | Missense_Mutation |
| NM_000546 | 44973 | c.516T > C | p.V172V | Loss of Function | Synonymous_Mutation |
| ENST00000545858 | 220781 | c.194G > A | p.R65H | Loss of Function | Missense_Mutation |
| NM_000546 | 43732 | c.517delG | p.V173fs*1 | Loss of Function | Frame_Shift_Del |
| NM_000546 | 45583 | c.514_559del46 | p.V173fs*59 | Loss of Function | Frame_Shift_Del |
| NM_000546 | 43054 | c.518T > G | p.V173G | Loss of Function | Missense_Mutation |
| NM_000546 | 44383 | c.518T > G | p.V173G | Loss of Function | Missense_Mutation |
| NM_000546 | 43559 | c.517G > T | p.V173L | Loss of Function | Missense_Mutation |
| NM_000546 | 44057 | c.517G > C | p.V173L | Loss of Function | Missense_Mutation |
| NM_000546 | 11084 | c.517G > A | p.V173M | Loss of Function | Missense_Mutation |
| NM_000546 | 44517 | c.519G > A | p.V173V | Loss of Function | Synonymous_Mutation |
| NM_000546 | 44018 | c.214C > T | p.R72C | Loss of Function | Missense_Mutation |
| NM_000546 | 43905 | c.590T > G | p.V197G | Loss of Function | Missense_Mutation |
| NM_000546 | 45985 | c.215G > A | p.R72H | Loss of Function | Missense_Mutation |
| ENST00000414315 | 241999 | c.242G > T | p.R81L | Loss of Function | Missense_Mutation |
| NM_000546 | 44845 | c.591G > A | p.V197V | Loss of Function | Synonymous_Mutation |
| ENST00000545858 | 99024 | c.245G > A | p.R82H | Loss of Function | Missense_Mutation |
| NM_000546 | 45308 | c.607delG | p.V203fs*44 | Loss of Function | Frame_Shift_Del |
| NM_000546 | 44226 | c.380C > T | p.S127F | Loss of Function | Missense_Mutation |
| NM_000546 | 45015 | c.380_381CC > TT | p.S127F | Loss of Function | Missense_Mutation |
| NM_000546 | 44707 | c.609G > A | p.V203V | Loss of Function | Synonymous_Mutation |
| NM_000546 | 53285 | c.379T > A | p.S127T | Loss of Function | Missense_Mutation |
| NM_000546 | 44282 | c.496T > G | p.S166A | Loss of Function | Missense_Mutation |
| NM_000546 | 44274 | c.647T > A | p.V216E | Loss of Function | Missense_Mutation |
| NM_000546 | 44239 | c.647delT | p.V216fs*31 | Loss of Function | Frame_Shift_Del |
| NM_000546 | 43681 | c.647T > G | p.V216G | Loss of Function | Missense_Mutation |
| NM_000546 | 11210 | c.646G > T | p.V216L | Loss of Function | Missense_Mutation |
| NM_000546 | 10667 | c.646G > A | p.V216M | Loss of Function | Missense_Mutation |
| NM_000546 | 44289 | c.497C > T | p.S166L | Loss of Function | Missense_Mutation |
| NM_000546 | 44035 | c.496T > C | p.S166P | Loss of Function | Missense_Mutation |
| NM_000546 | 44300 | c.548C > T | p.S183L | Loss of Function | Missense_Mutation |
| NM_000546 | 44343 | c.547T > C | p.S183P | Loss of Function | Missense_Mutation |
| NM_000546 | 44714 | c.553A > G | p.S185G | Loss of Function | Missense_Mutation |
| NM_000546 | 44185 | c.555C > A | p.S185R | Loss of Function | Missense_Mutation |
| NM_000546 | 45198 | c.555C > T | p.S185S | Loss of Function | Missense_Mutation |
| NM_000546 | 44198 | c.653T > G | p.V218G | Loss of Function | Missense_Mutation |
| NM_000546 | 11307 | c.643A > T | p.S215C | Loss of Function | Missense_Mutation |
| NM_000546 | 44093 | c.644G > A | p.S215N | Loss of Function | Missense_Mutation |
| NM_000546 | 45511 | c.645T > C | p.S215S | Loss of Function | Missense_Mutation |
| NM_000546 | 13421 | c.814delG | p.V272fs*73 | Loss of Function | Frame_Shift_Del |
| NM_000546 | 44870 | c.815T > G | p.V272G | Loss of Function | Missense_Mutation |
| NM_000546 | 44175 | c.644G > C | p.S215T | Loss of Function | Missense_Mutation |
| NM_000546 | 43920 | c.680C > T | p.S227F | Loss of Function | Missense_Mutation |
| NM_000546 | 10891 | c.814G > A | p.V272M | Loss of Function | Missense_Mutation |
| NM_000546 | 44393 | c.821T > C | p.V274A | Loss of Function | Missense_Mutation |
| NM_000546 | 44448 | c.821T > A | p.V274D | Loss of Function | Missense_Mutation |
| NM_000546 | 10769 | c.820G > T | p.V274F | Loss of Function | Missense_Mutation |
| NM_000546 | 43945 | c.821T > G | p.V274G | Loss of Function | Missense_Mutation |
| NM_000546 | 44621 | c.718A > T | p.S240C | Loss of Function | Missense_Mutation |
| NM_000546 | 44443 | c.820G > C | p.V274L | Loss of Function | Missense_Mutation |
| NM_000546 | 45491 | c.822T > G | p.V274V | Loss of Function | Synonymous_Mutation |
| NM_000546 | 43660 | c.719G > T | p.S240I | Loss of Function | Missense_Mutation |
| NM_000546 | 43684 | c.720T > G | p.S240R | Loss of Function | Missense_Mutation |
| NM_000546 | 44192 | c.272G > A | p.W91* | Loss of Function | Nonsense_Mutation |
| NM_000546 | 44492 | c.273G > A | p.W91* | Loss of Function | Nonsense_Mutation |
| NM_000546 | 44453 | c.309C > G | p.Y103* | Loss of Function | Nonsense_Mutation |
| NM_000546 | 11448 | c.321C > G | p.Y107* | Loss of Function | Nonsense_Mutation |
| NM_000546 | 45040 | c.321C > A | p.Y107* | Loss of Function | Nonsense_Mutation |
| NM_000546 | 46103 | c.319T > G | p.Y107D | Loss of Function | Missense_Mutation |
| NM_000546 | 45509 | c.321C > T | p.Y107Y | Loss of Function | Synonymous_Mutation |
| NM_000546 | 10862 | c.378C > G | p.Y126* | Loss of Function | Nonsense_Mutation |
| NM_000546 | 10888 | c.378C > A | p.Y126* | Loss of Function | Nonsense_Mutation |
| NM_000546 | 45261 | c.720T > A | p.S240R | Loss of Function | Missense_Mutation |
| NM_000546 | 44964 | c.719G > C | p.S240T | Loss of Function | Missense_Mutation |
| NM_000546 | 11517 | c.377A > G | p.Y126C | Loss of Function | Splice_Site |
| NM_000546 | 43900 | c.376T > G | p.Y126D | Loss of Function | Splice_Site |
| NM_000546 | 249845 | c.377_377delA | p.Y126fs*44 | Loss of Function | Frame_Shift_Del |
| NM_000546 | 44380 | c.376T > A | p.Y126N | Loss of Function | Splice_Site |
| NM_000546 | 44142 | c.377A > C | p.Y126S | Loss of Function | Splice_Site |
| NM_000546 | 43820 | c.489C > G | p.Y163* | Loss of Function | Nonsense_Mutation |
| NM_000546 | 45411 | c.489C > A | p.Y163* | Loss of Function | Nonsence_Mutation |
| NM_000546 | 10808 | c.488A > G | p.Y163C | Loss of Function | Missense_Mutation |
| NM_000546 | 44216 | c.487T > G | p.Y163D | Loss of Function | Missense_Mutation |
| NM_000546 | 45194 | c.487delT | p.Y163fs*7 | Loss of Function | Frame_Shift_Del |
| NM_000546 | 43846 | c.487T > C | p.Y163H | Loss of Function | Missense_Mutation |
| NM_000546 | 44623 | c.487T > A | p.Y163N | Loss of Function | Missense_Mutation |
| NM_000546 | 44224 | c.721T > G | p.S241A | Loss of Function | Missense_Mutation |
| NM_000546 | 44391 | c.489C > T | p.Y163Y | Loss of Function | Synonymous_Mutation |
| NM_000546 | 43928 | c.615T > A | p.Y205* | Loss of Function | Nonsense_Mutation |
| NM_000546 | 44924 | c.615T > G | p.Y205* | Loss of Function | Nonsense_Mutation |
| NM_000546 | 43947 | c.614A > G | p.Y205C | Loss of Function | Missense_Mutation |
| NM_000546 | 45168 | c.722_724delCCT | p.S241del | Loss of Function | In_Frame_Del |
| NM_000546 | 45548 | c.721_723delTCC | p.S241del | Loss of Function | In_Frame_Del |
| NM_000546 | 44067 | c.721T > A | p.S241T | Loss of Function | Missense_Mutation |
| NM_000546 | 45685 | c.613T > A | p.Y205N | Loss of Function | Missense_Mutation |
| NM_000546 | 146240 | c.806_808delGCT | p.S269_F270 > I | Loss of Function | In_Frame_Del |
| NM_000546 | 44505 | c.660T > G | p.Y220* | Loss of Function | Nonsense_Mutation |
| NM_000546 | 10758 | c.659A > G | p.Y220C | Loss of Function | Missense_Mutation |
| NM_000546 | 45248 | c.805A > T | p.S269C | Loss of Function | Missense_Mutation |
| NM_000546 | 44585 | c.655delC | p.Y220fs*27 | Loss of Function | Frame_Shift_Del |
| NM_000546 | 44637 | c.658T > C | p.Y220H | Loss of Function | Missense_Mutation |
| NM_000546 | 43962 | c.805A > G | p.S269G | Loss of Function | Missense_Mutation |
| NM_000546 | 43850 | c.659A > C | p.Y220S | Loss of Function | Missense_Mutation |
| NM_000546 | 45114 | c.702C > A | p.Y234* | Loss of Function | Nonsense_Mutation |
| NM_000546 | 10725 | c.701A > G | p.Y234C | Loss of Function | Missense_Mutation |
| NM_000546 | 44236 | c.806G > A | p.S269N | Loss of Function | Missense_Mutation |
| NM_000546 | 44886 | c.807C > T | p.S269S | Loss of Function | Missense_Mutation |
| NM_000546 | 45507 | c.806G > C | p.S269T | Loss of Function | Missense_Mutation |
| NM_000546 | 43956 | c.700T > A | p.Y234N | Loss of Function | Missense_Mutation |
| NM_000546 | 43865 | c.701A > C | p.Y234S | Loss of Function | Missense_Mutation |
| NM_000546 | 43564 | c.708C > A | p.Y236* | Loss of Function | Nonsense_Mutation |
| NM_000546 | 44960 | c.708C > G | p.Y236* | Loss of Function | Nonsense_Mutation |
| NM_000546 | 10731 | c.707A > G | p.Y236C | Loss of Function | Missense_Mutation |
| NM_000546 | 43602 | c.706T > G | p.Y236D | Loss of Function | Missense_Mutation |
| NM_000546 | 44565 | c.907A > T | p.S303C | Loss of Function | Missense_Mutation |
| NM_000546 | 43986 | c.908G > A | p.S303N | Loss of Function | Missense_Mutation |
| NM_000546 | 43826 | c.706T > A | p.Y236N | Loss of Function | Missense_Mutation |
| NM_000546 | 44167 | c.908G > C | p.S303T | Loss of Function | Missense_Mutation |
| NM_000546 | 44132 | c.708C > T | p.Y236Y | Loss of Function | Synonymous_Mutation |
| ENST00000269305 | 131534 | c.559 + 1G > A | p.? | Loss of Function | N/A |
| NM_000546 | 44832 | c.1096T > G | p.S366A | Loss of Function | Missense_Mutation |
| ENST00000269305 | 179823 | c.528C > A | p.C176* | Loss of Function | Nonsense_Mutation |
| NM_000546 | 44048 | c.280T > A | p.S94T | Loss of Function | Missense_Mutation |
| NM_000546 | 44673 | c.284C > T | p.S95F | Loss of Function | Missense_Mutation |
| NM_000546 | 44447 | c.287C > T | p.S96F | Loss of Function | Missense_Mutation |
| NM_000546 | 44036 | c.296C > T | p.S99F | Loss of Function | Missense_Mutation |
| ENST00000269305 | 118013 | c.592G > T | p.E198* | Loss of Function | Nonsense_Mutation |
| NM_000546 | 43678 | c.305C > T | p.T102I | Loss of Function | Missense_Mutation |
| NM_000546 | 44552 | c.509C > T | p.T170M | Loss of Function | Missense_Mutation |
| ENST00000269305 | 126981 | c.880G > T | p.E294* | Loss of Function | Nonsense_Mutation |
| NM_000546 | 44238 | c.631A > G | p.T211A | Loss of Function | Missense_Mutation |
| NM_000546 | 44661 | c.632C > A | p.T211N | Loss of Function | Missense_Mutation |
| NM_000546 | 43868 | c.689C > T | p.T230I | Loss of Function | Missense_Mutation |
| ENST00000269305 | 111498 | c.532delC | p.H178fs*69 | Loss of Function | Frame_Shift_Del |
| NM_000546 | 43806 | c.689C > A | p.T230N | Loss of Function | Missense_Mutation |
| NM_000546 | 45631 | c.688A > T | p.T230S | Loss of Function | Missense_Mutation |
| NM_000546 | 43980 | c.691A > G | p.T231A | Loss of Function | Missense_Mutation |
| NM_000546 | 44820 | c.692C > T | p.T231I | Loss of Function | Missense_Mutation |
| NM_000546 | 43889 | c.691A > T | p.T231S | Loss of Function | Missense_Mutation |
| NM_000546 | 45322 | c.757A > G | p.T253A | Loss of Function | Missense_Mutation |
| NM_000546 | 43683 | c.758C > T | p.T253I | Loss of Function | Missense_Mutation |
| NM_000546 | 45980 | c.757A > C | p.T253P | Loss of Function | Missense_Mutation |
| ENST00000269305 | 117949 | c.574C > T | p.Q192* | Loss of Function | Nonsense_Mutation |
| NM_000546 | 43881 | c.757A > T | p.T253S | Loss of Function | Missense_Mutation |
| NM_000546 | 44544 | c.766A > G | p.T256A | Loss of Function | Missense_Mutation |
| NM_000546 | 44662 | c.766A > T | p.T256S | Loss of Function | Missense_Mutation |
| ENST00000269305 | 99668 | c.586C > T | p.R196* | Loss of Function | Nonsense_Mutation |
| ENST00000269305 | 99618 | c.637C > T | p.R213* | Loss of Function | Nonsense_Mutation |
| NM_000546 | 45728 | c.850A > G | p.T284A | Loss of Function | Missense_Mutation |
| NM_000546 | 46207 | c.910A > G | p.T304A | Loss of Function | Missense_Mutation |
| NM_000546 | 45128 | c.911C > T | p.T304I | Loss of Function | Missense_Mutation |
| NM_000546 | 44200 | c.242C > T | p.T81I | Loss of Function | Missense_Mutation |
| NM_000546 | 44329 | c.470T > A | p.V157D | Loss of Function | Missense_Mutation |
| NM_000546 | 45551 | c.469_471delGTC | p.V157del | Loss of Function | In_Frame_Del |
| ENST00000269305 | 131480 | c.469G > T | p.V157F | Loss of Function | Missense_Mutation |
| ENST00000413465 | 131481 | c.469G > T | p.V157F | Loss of Function | Missense_Mutation |
| NM_000546 | 43625 | c.469G > A | p.V157I | Loss of Function | Missense_Mutation |
| ENST00000269305 | 99947 | c.916C > T | p.R306* | Loss of Function | Nonsense_Mutation |
| ENST00000269305 | 99721 | c.1024C > T | p.R342* | Loss of Function | Nonsense_Mutation |
| NM_000546 | 45120 | c.469G > C | p.V157L | Loss of Function | Missense_Mutation |
| NM_000546 | 44996 | c.515T > C | p.V172A | Loss of Function | Missense_Mutation |
| NM_000546 | 44229 | c.515T > A | p.V172D | Loss of Function | Missense_Mutation |
| NM_000546 | 43955 | c.514G > A | p.V172I | Loss of Function | Missense_Mutation |
| NM_000546 | 44327 | c.518T > C | p.V173A | Loss of Function | Missense_Mutation |
| ENST00000269305 | 121042 | c.517G > C | p.V173L | Loss of Function | Missense_Mutation |
| ENST00000269305 | 99946 | c.378C > G | p.Y126* | Loss of Function | Nonsense_Mutation |
| ENST00000269305 | 99641 | c.517G > T | p.V173L | Loss of Function | Missense_Mutation |
| ENST00000413465 | 121043 | c.517G > C | p.V173L | Loss of Function | Missense_Mutation |
| ENST00000413465 | 99638 | c.517G > T | p.V173L | Loss of Function | Missense_Mutation |
| ENST00000413465 | 98964 | c.517G > A | p.V173M | Loss of Function | Missense_Mutation |
| NM_000546 | 44424 | c.590T > A | p.V197E | Loss of Function | Missense_Mutation |
| ENST00000413465 | 131535 | c.559 + 1G > A | p.? | Loss of Function | N/A |
| NM_0000546 | 46212 | c.589G > T | p.V197L | Loss of Function | Missense_Mutation |
| ENST00000413465 | 179822 | c.528C > A | p.C176* | Loss of Function | Nonsense_Mutation |
| NM_000546 | 43779 | c.589G > A | p.V197M | Loss of Function | Missense_Mutation |
| NM_000546 | 44411 | c.608T > A | p.V203E | Loss of Function | Missense_Mutation |
| ENST00000413465 | 118010 | c.592G > T | p.E198* | Loss of Function | Nonsense_Mutation |
| NM_000546 | 44365 | c.607G > T | p.V203L | Loss of Function | Missense_Mutation |
| NM_000546 | 43599 | c.607G > A | p.V203M | Loss of Function | Missense_Mutation |
| ENST00000413465 | 111495 | c.532delC | p.H178fs*69 | Loss of Function | Frame_Shift_Del |
| NM_000546 | 44567 | c.647T > C | p.V216A | Loss of Function | Missense_Mutation |
| NM_000546 | 44607 | c.646_648delGTG | p.V216del | Loss of Function | In_Frame_Del |
| NM_000546 | 45110 | c.650T > C | p.V217A | Loss of Function | Missense_Mutation |
| NM_000546 | 44929 | c.650T > A | p.V217E | Loss of Function | Missense_Mutation |
| NM_000546 | 44375 | c.650T > G | p.V217G | Loss of Function | Missense_Mutation |
| ENST00000413465 | 117946 | c.574C > T | p.Q192* | Loss of Function | Nonsense_Mutation |
| NM_000546 | 44334 | c.649G > T | p.V217L | Loss of Function | Missense_Mutation |
| NM_000546 | 44930 | c.653T > C | p.V218A | Loss of Function | Missense_Mutation |
| NM_000546 | 6496 | c.652_654delGTG | p.V218del | Loss of Function | In_Frame_Del |
| ENST00000413465 | 99665 | c.586C > T | p.R196* | Loss of Function | Nonsense_Mutation |
| ENST00000413465 | 99615 | c.637C > T | p.R213* | Loss of Function | Nonsense Mutation |
| NM_000546 | 44317 | c.653T > A | p.V218E | Loss of Function | Missense_Mutation |
| NM_000546 | 44683 | c.652G > A | p.V218M | Loss of Function | Missense_Mutation |
| ENST00000414315 | 131483 | c.73G > T | p.V25F | Loss of Function | Missense_Mutation |
| NM_000546 | 44294 | c.815T > C | p.V272A | Loss of Function | Missense_Mutation |
| NM_000546 | 44580 | c.815T > A | p.V272E | Loss of Function | Missense_Mutation |
| NM_000546 | 10859 | c.814G > T | p.V272L | Loss of Function | Missense_Mutation |
| NM_000546 | 45898 | c.814G > C | p.V272L | Loss of Function | Missense_Mutation |
| ENST00000269305 | 99950 | c.814G > A | p.V272M | Loss of Function | Missense_Mutation |
| ENST00000269305 | 165075 | c.820G > T | p.V274F | Loss of Function | Missense_Mutation |
| ENST00000413465 | 99944 | c.378C > G | p.Y126* | Loss of Function | Nonsense_Mutation |
| NM_000546 | 43667 | c.820G > A | p.V274I | Loss of Function | Missense_Mutation |
| ENST00000414315 | 121045 | c.121G > C | p.V41L | Loss of Function | Missense_Mutation |
| ENST00000414315 | 99639 | c.121G > T | p.V41L | Loss of Function | Missense_Mutation |
| ENST00000414315 | 98965 | c.121G > A | p.V41M | Loss of Function | Missense_Mutation |
| ENST00000545858 | 131482 | c.190G > T | p.V64F | Loss of Function | Missense_Mutation |
| ENST00000414315 | 131537 | c.163 + 1G > A | p.? | Loss of Function | N/A |
| NM_000546 | 45288 | c.217G > C | p.V73L | Loss of Function | Missense_Mutation |
| ENST00000414315 | 179824 | c.132C > A | p.C44* | Loss of Function | Nonsense_Mutation |
| NM_000546 | 43787 | c.217G > A | p.V73M | Loss of Function | Missense_Mutation |
| ENST00000414315 | 118011 | c.196G > T | p.E66* | Loss of Function | Nonsense_Mutation |
| ENST00000414315 | 111496 | c.136delC | p.H46fs* > 45 | Loss of Function | Frame_Shift_Del |
| ENST00000545858 | 121044 | c.238G > C | p.V80L | Loss of Function | Missense_Mutation |
| ENST00000545858 | 99640 | c.238G > T | p.V80L | Loss of Function | Missense_Mutation |
| ENST00000545858 | 98966 | c.238G > A | p.V80M | Loss of Function | Missense_Mutation |
| ENST00000269305 | 220766 | c.319T > G | p.Y107D | Loss of Function | Missense_Mutation |
| ENST00000414315 | 117947 | c.178C > T | p.Q60* | Loss of Function | Nonsense_Mutation |
| ENST00000413465 | 220765 | c.319T > G | p.Y107D | Loss of Function | Missense_Mutation |
| NM_000546 | 44405 | c.376_396del21 | p.Y126_K132delYSP | Loss of Function | In_Frame_Del |
| ENST00000414315 | 99666 | c.190C > T | p.R64* | Loss of Function | Nonsense_Mutation |
| ENST00000414315 | 99616 | c.241C > T | p.R81* | Loss of Function | Nonsense_Mutation |
| NM_000546 | 44774 | c.376_393del18 | p.Y126_N131delYSP | Loss of Function | In_Frame_Del |
| ENST00000269305 | 220783 | c.376T > G | p.Y126D | Loss of Function | Missense_Mutation |
| ENST00000413465 | 220782 | c.376T > G | p.Y126D | Loss of Function | Missense_Mutation |
| ENST00000545858 | 99719 | c.380A > G | p.Y127C | Loss of Function | Missense_Mutation |
| ENST00000545858 | 165074 | c.422A > G | p.Y141C | Loss of Function | Missense_Mutation |
| ENST00000545858 | 116673 | c.428A > G | p.Y143C | Loss of Function | Missense_Mutation |
| ENST00000545858 | 131536 | c.280 + 1G > A | p.? | Loss of Function | N/A |
| ENST00000269305 | 129852 | c.488A > G | p.Y163C | Loss of Function | Missense_Mutation |
| ENST00000413465 | 129853 | c.488A > G | p.Y163C | Loss of Function | Missense_Mutation |
| ENST00000545858 | 179825 | c.249C > A | p.C83* | Loss of Function | Nonsense Mutation |
| NM_000546 | 45025 | c.488A > C | p.Y163S | Loss of Function | Missense_Mutation |
| ENST00000545858 | 118012 | c.313G > T | p.E105* | Loss of Function | Nonsense_Mutation |
| NM_000546 | 43844 | c.613T > G | p.Y205D | Loss of Function | Missense_Mutation |
| NM_000546 | 11351 | c.614A > T | p.Y205F | Loss of Function | Missense_Mutation |
| NM_000546 | 43642 | c.613T > C | p.Y205H | Loss of Function | Missense_Mutation |
| ENST00000545858 | 111497 | c.253delC | p.H85fs*69 | Loss of Function | Frame_Shift_Del |
| NM_000546 | 44169 | c.614A > C | p.Y205S | Loss of Function | Missense_Mutation |
| ENST00000269305 | 99720 | c.659A > G | p.Y220C | Loss of Function | Missense_Mutation |
| ENST00000413465 | 99718 | c.659A > G | p.Y220C | Loss of Function | Missense_Mutation |
| NM_000546 | 11847 | c.658T > G | p.Y220D | Loss of Function | Missense_Mutation |
| ENST00000545858 | 117948 | c.295C > T | p.Q99* | Loss of Function | Nonsense_Mutation |
| ENST00000545858 | 99667 | c.307C > T | p.R103* | Loss of Function | Nonsense_Mutation |
| ENST00000545858 | 99617 | c.358C > T | p.R120* | Loss of Function | Nonsense_Mutation |
| NM_000546 | 44672 | c.658T > A | p.Y220N | Loss of Function | Missense_Mutation |
| ENST00000269305 | 165073 | c.701A > G | p.Y234C | Loss of Function | Missense_Mutation |
| ENST00000413465 | 165072 | c.701A > G | p.Y234C | Loss of Function | Missense_Mutation |
| NM_000546 | 43768 | c.700T > G | p.Y234D | Loss of Function | Missense_Mutation |
| NM_000546 | 44953 | c.700_702delTAC | p.Y234del | Loss of Function | In_Frame_Del |
| NM_000546 | 11152 | c.700T > C | p.Y234H | Loss of Function | Missense_Mutation |
| ENST00000269305 | 116674 | c.707A > G | p.Y236C | Loss of Function | Missense_Mutation |
| ENST00000413465 | 116672 | c.707A > G | p.Y236C | Loss of Function | Missense_Mutation |
| NM_000546 | 44072 | c.706_708delTAC | p.Y236del | Loss of Function | In_Frame_Del |
| NM_000546 | 44326 | c.706T > C | p.Y236H | Loss of Function | Missense_Mutation |
| NM_000546 | 44693 | c.707A > C | p.Y236S | Loss of Function | Missense_Mutation |
| ENST00000414315 | 129855 | c.92A > G | p.Y31C | Loss of Function | Missense_Mutation |
| ENST00000545858 | 99945 | c.99C > G | p.Y33* | Loss of Function | Nonsense_Mutation |
| ENST00000545858 | 220784 | c.97T > G | p.Y33D | Loss of Function | Missense_Mutation |
| ENST00000545858 | 129854 | c.209A > G | p.Y70C | Loss of Function | Missense_Mutation |

### Likelihood of Response to Immunotherapy

Various embodiments of this disclosure relate to a method of identifying a cancer patient as having an increased or reduced likelihood of responding to a cancer therapy, such as an immunotherapy. In some embodiments, the method comprises the following steps: (i)obtaining a biological sample from said patient and detecting whether the biological sample comprises a loss-of-function TP53 mutation; and (ii) identifying said patient as having an increased likelihood of response to the immunotherapy if the biological sample does not comprise the loss-of-function TP53 mutation and identifying said patient as having a reduced likelihood of response to the immunotherapy if the biological sample comprises the loss-of-function TP53 mutation.

An increased likelihood of responding to an immunotherapy is, in certain instances, a percent increase in the probability of the cancer patient demonstrating regression in response to the immunotherapy, wherein the percent increase is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%.

An increased likelihood of responding to an immunotherapy is, in certain instances, a percent increase in the probability of the cancer patient demonstrating prolonged tumor free survival (TFS) in response to the immunotherapy, wherein the percent increase is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%.

In some embodiments, an increased likelihood of responding to an immunotherapy, in a cancer patient, is an increase in the duration of time when said patient demonstrates tumor free survival (TFS). In some examples, the increase in the duration of time is at least about 2 weeks, at least about 4 weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, at least about 52 weeks, at least about 13 months, at least about 15 months, at least about 17 months, at least about 19 months, at least about 21 months, at least about 23 months, at least about 24 months, at least about 3 years, at least about 5 years, at least about 10 years, at least about 15 years, or at least about 20 years.

An increased likelihood of responding to an immunotherapy is, in certain instances, a percent increase in the probability of the cancer patient demonstrating prolonged progression free survival (PFS) in response to the immunotherapy, wherein the percent increase is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%.

In some embodiments, an increased likelihood of responding to an immunotherapy, in a cancer patient, is an increase in the duration of time when the patient demonstrates progression free survival (PFS). In some examples, the increase in the duration of time is at least about 2 weeks, at least about 4 weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, at least about 52 weeks, at least about 13 months, at least about 15 months, at least about 17 months, at least about 19 months, at least about 21 months, at least about 23 months, at least about 24 months, at least about 3 years, at least about 5 years, at least about 10 years, at least about 15 years, or at least about 20 years.

An increased likelihood of responding to an immunotherapy is, in certain instances, a percent increase in the probability of the cancer patient demonstrating prolonged overall survival (OS) in response to the immunotherapy, wherein the percent increase is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%.

In some embodiments, an increased likelihood of responding to an immunotherapy, in a cancer patient, is an increase in the length of time said patient is still alive, also referred to as overall survival (OS). In some examples, the increase in the length of time is at least about 2 weeks, at least about 4 weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, at least about 52 weeks, at least about 13 months, at least about 15 months, at least about 17 months, at least about 19 months, at least about 21 months, at least about 23 months, at least about 24 months, at least about 3 years, at least about 5 years, at least about 10 years, at least about 15 years, or at least about 20 years.

In yet other embodiments, an increased likelihood of responding to an immunotherapy, in a cancer patient, is a percent decrease in a probability of the cancer patient experiencing a relapse of a cancer or a tumor. In some examples, the percent decrease is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%.

In some embodiments, an increased likelihood of responding to an immunotherapy, in a cancer patient, is an increase in the length of time till said patient experiences a relapse of a cancer or tumor. In some examples, the increase in the length of time is at least about 2 weeks, at least about 4 weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, at least about 52 weeks, at least about 13 months, at least about 15 months, at least about 17 months, at least about 19 months, at least about 21 months, at least about 23 months, at least about 24 months, at least about 3 years, at least about 5 years, at least about 10 years, at least about 15 years, or at least about 20 years.

A reduced likelihood of responding to an immunotherapy, in a cancer patient, is in some embodiments, a percent decrease in the probability of the cancer patient demonstrating regression in response to the immunotherapy, wherein the percent decrease is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%.

In some embodiments, a reduced likelihood of responding to an immunotherapy, in a cancer patient, is a decrease in the duration of time when said patient demonstrates tumor free survival (TFS). In some examples, the decrease in the duration of time is at least about 2 weeks, at least about 4 weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, at least about 52 weeks, at least about 13 months, at least about 15 months, at least about 17 months, at least about 19 months, at least about 21 months, at least about 23 months, at least about 24 months, at least about 3 years, at least about 5 years, at least about 10 years, at least about 15 years, or at least about 20 years.

A reduced likelihood of responding to an immunotherapy is, in certain instances, a percent decrease in the probability of the cancer patient demonstrating prolonged progression free survival (PFS) in response to the immunotherapy, wherein the percent increase is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%.

In some embodiments, a reduced likelihood of responding to an immunotherapy, in a cancer patient, is a decrease in the duration of time when the patient demonstrates progression free survival (PFS). In some examples, the decrease in the duration of time is at least about 2 weeks, at least about 4 weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, at least about 52 weeks, at least about 13 months, at least about 15 months, at least about 17 months, at least about 19 months, at least about 21 months, at least about 23 months, at least about 24 months, at least about 3 years, at least about 5 years, at least about 10 years, at least about 15 years, or at least about 20 years.

A reduced likelihood of responding to an immunotherapy is, in certain instances, a percent decrease in the probability of the cancer patient demonstrating prolonged overall survival (OS) in response to the immunotherapy, wherein the percent decrease is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%.

In some embodiments, a reduced likelihood of responding to an immunotherapy, in a cancer patient, is a decrease in the length of time said patient is still alive, also referred to as overall survival (OS). In some examples, the decrease in the length of time is at least about 2 weeks, at least about 4 weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, at least about 52 weeks, at least about 13 months, at least about 15 months, at least about 17 months, at least about 19 months, at least about 21 months, at least about 23 months, at least about 24 months, at least about 3 years, at least about 5 years, at least about 10 years, at least about 15 years, or at least about 20 years.

In yet other embodiments, a reduced likelihood of responding to an immunotherapy, in a cancer patient, is, a percent increase in a probability of the cancer patient experiencing a relapse of a cancer or a tumor. In some examples, the percent increase is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%.

In some embodiments, a reduced likelihood of responding to an immunotherapy, in a cancer patient, is a decrease in the length of time till said patient experiences a relapse of a cancer or tumor. In some examples, the decrease in the length of time is at least about 2 weeks, at least about 4 weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, at least about 52 weeks, at least about 13 months, at least about 15 months, at least about 17 months, at least about 19 months, at least about 21 months, at least about 23 months, at least about 24 months, at least about 3 years, at least about 5 years, at least about 10 years, at least about 15 years, or at least about 20 years.

In additional embodiments, a method of identifying a cancer patient as having a reduced likelihood of responding to an immunotherapy, by determining the presence of a loss-of-function TP53 mutation, and not administering the immunotherapy to patients identified as having reduced likelihood of response, is correlated with an overall reduction in the percentage of cancer patients who are exposed to various side effects of immunotherapy without getting a therapeutic benefit. For instance, it has been shown that intravenous infusion of anti-CD40 results in widespread systemic exposure to the immunoagonist, leading to symptoms of cytokine release syndrome (fever, headaches, nausea, chills), noninfectious ocular inflammation, elevated hepatic enzymes (indicative of liver damage), and hematologic toxicities including T-cell depletion. See Kwong et al. Induction of potent anti-tumor responses while eliminating systemic side effects via liposome-anchored combinatorial immunotherapy, Biomaterials. 2011 Aug; 32(22): 5134-5147. Thus, in some embodiments of the present disclosure, identifying a cancer patient as having a reduced likelihood of responding to an immunotherapy, by determining the presence of a loss-of-function TP53 mutation, and not administering the immunotherapy to patients identified as having reduced likelihood of response, is correlated with an overall reduction in the percentage of cancer patients who are exposed to systemic side effects associated with immunotherapy, without getting a therapeutic benefit.

In some instances, a method of identifying a cancer patient as having an increased likelihood of responding to an immunotherapy comprises assaying the levels of one or more of MHC-I, ERAP1, TAP1 in a tumor sample isolated from said cancer patient. In some cases, levels of all three proteins are determined simultaneously. In other cases, level of only one of the three proteins is determined in an assay and said level of only one of the three proteins is sufficient to identify the cancer patient as having an increased or a reduced likelihood of responding to the immunotherapy. In yet other cases, levels of all three proteins are determined sequentially, for example, MHC-I followed by ERAP1 followed by TAP1, or ERAP1 followed by TAP1 followed by MHC-I, or TAP1 followed by ERAP1 followed by MHC-I, or MHC-I followed by simultaneous detection of ERAP1 and TAP1, or simultaneous detection of ERAP1 and TAP 1 followed by MHC-I. In some examples, only MHC-I level is assessed. In some examples, only ERAP1 level is assessed. In some examples, only TAP1 level is assessed.

The tumor sample levels of one or more of MHC-I, ERAP1, and TAP1 are compared to that in a reference non-tumor biological sample. In some cases, the reference non-tumor biological sample is from the same patient. In other cases, the reference non-tumor biological sample is from another subject who does not have cancer. The reference non-tumor biological sample is, in certain embodiments, a liquid sample or a tissue sample. In some embodiments, the liquid sample is blood.

The detection of a loss-of-function TP53 mutation, in certain embodiments, is carried out in combination with assaying the levels of one or more of MHC-I, ERAP1, and TAP1. For instance, a tumor sample from a cancer patient is first analyzed to detect the presence or absence of the loss-of-function TP53 mutation and subsequently the levels of one or more of MHC-I, ERAP1, and TAP1 in said tumor sample is assayed. Alternately, a tumor sample from a cancer patient is first analyzed to determine the levels of one or more of MHC-I, ERAP1, and TAP1 and subsequently presence or absence of the loss-of-function TP53 mutation is determined in said tumor sample.

In various embodiments, the outcome of the detection of the TP53 loss-of-function mutation in the tumor sample and the levels of one or more of MHC-I, ERAP1, and TAP1 in the tumor sample, compared to that in a reference non-tumor biological sample is correlated with identifying the cancer patient as having an increased or a reduced likelihood of responding to an immunotherapy. For instance, a patient whose tumor sample comprises a loss-of-function TP53 mutation or has a lower level of one or more of MHC-I, ERAP1, and TAP1 compared to a reference non-tumor biological sample is identified as having a reduced likelihood of responding to an immunotherapy. In another example, a patient whose tumor sample does not comprise a loss-of-function TP53 mutation or has comparable levels of one or more of MHC-I, ERAP1, and TAP1 as in a reference non-tumor biological sample, is identified as having an increased likelihood of responding to an immunotherapy. Comparable levels comprise, in some cases, values that are within about 10% to about 15% of each other.

### Immunotherapy

In various embodiments, immunotherapy comprises the destruction of tumor cells by a direct effect or by indirectly stimulating immune responses. An exemplary strategy, in some instances, is to take advantage of soluble molecules, such as cytokines which are independent of antigen recognition by host immune cells (e.g., IL-2, IFN-α, IL-7, GM-CSF). In some embodiments, immunotherapy comprises targeting immune molecular checkpoints using checkpoint receptor inhibitors, such as anti-T-lymphocyte-associated antigen 4 (CTLA-4), anti-Programmed Cell Death 1 (PD-1) antibodies, anti-T-cell immunoglobulin domain and mucin domain-3 (TIM-3), and anti-lymphocyte activation gene 3 (LAG3).

In some examples, the immunotherapy comprises an immune checkpoint activator, such as an agonist of costimulation by CD27 (e.g., an agonist antibody that binds to CD27), an agonist of costimulation by CD40 (e.g., an agonist antibody 10 that binds to CD40), an agonist of costimulation by OX40 (e.g., an agonist antibody that binds to OX40), an agonist of costimulation by GITR (e.g., an agonist antibody that binds to GITR), an agonist of costimulation by CD137 (e.g., an agonist antibody that binds to CD137), an agonist of costimulation by CD28 (e.g., an agonist antibody that binds to CD28), an agonist of costimulation by ICOS (e.g., an agonist antibody that binds to ICOS).

In some examples, the immunotherapy comprises an immune checkpoint inhibitor, such as an antagonist of PD-1 (e.g., an antagonist antibody that binds to PD-1), an antagonist of PD-L1 (e.g., an antagonist antibody that binds to PD-L1), an antagonist of CTLA-4 (e.g., an antagonist antibody that binds to CTLA-4), an antagonist of A2AR (e.g., an antagonist antibody that binds to A2AR), an antagonist of B7-H3 (e.g., an antagonist antibody that binds to B7-H3), an antagonist of B7-H4 (e.g., an antagonist antibody that binds to B7-H4), an antagonist of BTLA (e.g., an antagonist antibody that binds to BTLA), an antagonist of IDO (e.g., an antagonist antibody that binds to IDO), an antagonist of KIR (e.g., an antagonist antibody that binds to KIR), an antagonist of LAG3 (e.g., an antagonist antibody that binds to LAG3), an antagonist of TIM-3 (e.g., an antagonist antibody that binds to TIM3).

In some embodiments, the immunotherapy comprises an immune checkpoint regulator. In one example, the immune checkpoint regulator is TGN1412. In one example, the immune checkpoint regulator is NKTR-214. In one example, the immune checkpoint regulator is MEDI0562. In one example, the immune checkpoint regulator is MEDI6469. In one example, the immune checkpoint regulator is MEDI6383. In one example, the immune checkpoint regulator is JTX-2011. In one example, the immune checkpoint regulator is Keytruda (pembrolizumab). In one example, the immune checkpoint regulator is Opdivo (nivolumab). In one example, the immune checkpoint regulator is Yervoy (ipilimumab). In one example, the immune checkpoint regulator is tremelimumab. In one example, the immune checkpoint regulator is Tecentriq (atezolizumab). In one example, the immune checkpoint regulator is MGA271. In one example, the immune checkpoint regulator is indoximod. In one example, the immune checkpoint regulator is Epacadostat. In one example, the immune checkpoint regulator is lirilumab. In one example, the immune checkpoint regulator is BMS-986016. In one example, the immune checkpoint regulator is MPDL3280A. In one example, the immune checkpoint regulator is avelumab. In one example, the immune checkpoint regulator is durvalumab. In one example, the immune checkpoint regulator is MEDI4736. In one example, the immune checkpoint regulator is MEDI4737. In one example, the immune checkpoint regulator is TRX518. In one example, the immune checkpoint regulator is MK-4166. In one example, the immune checkpoint regulator is urelumab (BMS-663513). In one example, the immune checkpoint regulator is PF-05082566 (PF-2566).

In some embodiments, the immune checkpoint inhibitor, activator, or regulator is administered by injection (such as subcutaneously or intravenously) at a dose (such as a flat dose) of about 100 mg to about 600 mg, about 200 mg to about 500 mg, about 100 mg to about 300 mg, about 250 mg to about 450 mg, about 300 mg to about 400 mg, about 250 mg to about 350 mg, about 350 mg to about 450 mg, or about 100 mg, about 200 mg, about 300 mg, or about 400 mg. The dosing schedule, such as a flat dosing schedule, in certain instances, varies from once a week to once every 2, 3, 4, 5, or 6 weeks. In one embodiment, the immune checkpoint inhibitor, activator, or regulator is administered at a dose of about 300 mg to 400 mg once every three weeks or once every four weeks. In some embodiments, the immune checkpoint inhibitor, activator, or regulator is administered twice weekly, once weekly, once every 2 weeks, once every 3 weeks, once every 4 weeks, once every 6 weeks, once every 2 months, once every 3 months, once every 4 months, once every 5 months, or once every 6 months. In one embodiment, the immune checkpoint inhibitor, activator, or regulator is administered at a dose of about 300 mg once every three weeks. In one embodiment, the immune checkpoint inhibitor, activator, or regulator is administered at a dose of about 400 mg once every four weeks. In one embodiment, the immune checkpoint inhibitor, activator, or regulator is administered at a dose of about 300 mg once every four weeks. In one embodiment, the immune checkpoint inhibitor, activator, or regulator is administered at a dose of about 400 mg once every three weeks. In certain embodiments, a typical dosage for an immune checkpoint inhibitor, activator, or regulator ranges from about 0.1 mg/kg to up to about 300 mg/kg or more. In certain embodiments, the dosage ranges from about 1 mg/kg up to about 300 mg/kg; or about 5 mg/kg up to about 300 mg/kg; or about 10 mg/kg up to about 300 mg/kg. In certain embodiments, a dosage for an immune checkpoint inhibitor, activator, or regulator, such as an immune checkpoint antibody ranges from about 1 mg/kg to up to about 1000 mg/kg or more, from about 5 mg/kg up to about 1000 mg/kg; or from about 10 mg/kg up to about 1000 mg/kg; or from about 50 mg/kg up to about 1000 mg/kg. It is understood that the dosage will depend upon the subject, the treatment regimen, the particular agent, the amount of side-effects tolerated, additional agents administered that counter the side effects and other such parameters.

For example, in some of methods described herein, the immune checkpoint inhibitor, activator, or regulator is administered in a dosage range that is from about 0.1 mg per kg body weight (mg/kg) to about 50 mg/kg, about 0.1 mg/kg to about 20 mg/kg, about 0.1 to about 10 mg/kg, about 0.3 to about 10 mg/kg, about 0.5 mg/kg to 5 mg/kg or 0.5 mg/kg to 1 mg/kg. Exemplary doses of an immune checkpoint inhibitor, activator, or regulator for use in any of the provided methods include a dosage that is at least or is at least about 0.1 mg/kg, at least about 0.15 mg/kg, at least about 0.2 mg/kg, at least about 0.25 mg/kg, at least about 0.30 mg/kg, at least about 0.35 mg/kg, at least about 0.40 mg/kg, at least about 0.45 mg/kg, at least about 0.5 mg/kg, at least about 0.55 mg.kg, at least about 0.6 mg/kg, at least about 0.7 mg/kg, at least about 0.8 mg/kg, at least about 0.9 mg/kg, at least about 1.0 mg/kg, at least about 1.1 mg/kg, at least about 1.2 mg/kg, at least about 1.3 mg/kg, at least about 1.4 mg/kg, at least about 1.5 mg/kg, at least about 1.6 mg/kg, at least about 1.7 mg/kg, at least about 1.8 mg/kg, at least about 1.9 mg/kg, at least about 2 mg/kg, at least about 2.5 mg/kg, at least about 3 mg/kg, at least about 3.5 mg/kg, at least about 4 mg/kg, at least about 4.5 mg/kg, at least about 5 mg/kg, at least about 5.5 mg/kg, at least about 6 mg/kg, at least about 6.5 mg/kg, at least about 7 mg/kg, at least about 7.5 mg/kg, at least about 8 mg/kg, at least about 8.5 mg/kg, at least about 9 mg/kg, at least about 9.5 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg, at least about 20 mg/kg, at least about 21 mg/kg, at least about 22 mg/kg, at least about 23 mg/kg, at least about 24 mg/kg, at least about 25 mg/kg, at least about 30 mg/kg, at least about 40 mg/kg, or at least about 50 mg/kg body weight of the subject to be treated.

In some embodiments, the immunotherapy comprises adoptive cell therapy. In some embodiments, the adoptive cell therapy is an adoptive T-cell therapy. In some cases, adoptive cell therapy comprises administration of adoptive cell therapeutic compositions. Examples of adoptive cell therapeutic compositions include, but are not limited to, compositions comprising a cell type selected from a group consisting of a tumor infiltrating lymphocyte (TIL), TCR (i.e. heterologous T-cell receptor) modified lymphocytes and CAR (i.e. chimeric antigen receptor) modified lymphocytes. In some embodiments, the adoptive cell therapeutic composition comprises a cell type selected from a group consisting of T-cells, CD8⁺ cells, CD4⁺ cells, NK-cells, delta-gamma T-cells, regulatory T-cells and peripheral blood mononuclear cells. In one embodiment, the adoptive cell therapeutic composition comprises T cells. In some examples, the adoptive cell therapy involves harvesting a patient's T cells, stimulating and expanding the cells that are capable of recognizing tumors, and then injecting these cells back into the patient so they can attack the tumor. In certain cases, isolated tumor infiltrating lymphocytes (TILs) are grown in culture to large numbers and infused into the patient. In another specific embodiment of the invention the adoptive cell therapeutic composition comprises T-cells which have been modified with target-specific chimeric antigen receptors or specifically selected T-cell receptors.

In certain instances, a lymphodepleting preparative regimen is administered prior to infusion of the adoptive cell therapeutic compositions. One example of a lymphodepleting preparative regimen comprises administering cyclophosphamide for a few days and fludarabine for a few days, followed by the adoptive cell therapeutic composition. In some embodiments, cyclophosphamide is administered at a concentration of 60 mg/kg for 2 days and fludarabine is administered at a concentration of 25 mg/m² for 5 days. In some embodiments, around 40-80 mg/kg, such as around 60 mg/kg of cyclophosphamide is administered for approximately 2 days after which around 15-35 mg/m², such as around 25 mg/m² fludarabine is administered for around five days. In some cases, the adoptive cell therapeutic composition is administered in combination with IL-2 IL-7, IL-15, IL-21, or combinations thereof, for example prior to, concurrently, or following the administration of the adoptive cell therapeutic composition.

The adoptive cell therapeutic composition is administered, in some embodiments, as an intra-arterial or intravenous infusion, which lasts about 30 to about 60 minutes. Other examples of routes of administration include intraperitoneal, intrathecal and intralymphatic. Any suitable dose of the adoptive cell therapeutic composition can be administered, such as, about 1×10¹⁰ lymphocytes to about 15×10¹⁰ lymphocytes, in some embodiments. In some embodiments, adoptive cell therapy comprises administering a composition comprising about 1×10³ lymphocytes to about 1×10¹² lymphocytes, from about 1×10⁴ lymphocytes to about 1×10¹⁰ lymphocytes, from about 1×10⁵ lymphocytes to about 1×10⁹ lymphocytes, from about 1×10⁶ lymphocytes to about 1×10⁸ lymphocytes, from about 1×10⁶ lymphocytes to about 1×10⁷ lymphocytes, from about 1×10⁷ lymphocytes to about 1×10⁸ lymphocytes, about 1×10⁵ lymphocytes, about 1×10⁶ lymphocytes, about 1×10⁷ lymphocytes, about 1×10⁸ lymphocytes, or about 1×10⁹ lymphocytes. Additional exemplary adoptive cell therapy incudes administering a composition comprising about 1×10³ lymphocytes to about 1×10¹² T-cells, from about 1×10⁴ T-cells to about 1×10¹⁰ T-cells, from about 1×10⁵ T-cells to about 1×10⁹ T-cells, from about 1×10⁶ T-cells to about 1×10⁸ T-cells, from about 1×10⁶ T-cells to about 1×10⁷ T-cells, from about 1×10⁷ T-cells to about 1×10⁸ T-cells, about 1×10⁵ T-cells, about 1×10⁶ T-cells, about 1×10⁷ T-cells, about 1×10⁸ T-cells, or about 1×10⁹ T-cells.

Dendritic cells (DCs) are specialized antigen-presenting cells with the unique capability to capture and process antigens, migrate from the periphery to a lymphoid organ, and present the antigens to resting T cells in a major histocompatibility complex (MHC)-restricted fashion (Banchereau, J. & Steinman, R. M. 1998. Nature 392:245-252; Steinman, R. M., et al. 2003. Ann Rev Immunol 21: 685-711, each of which is incorporated herein by reference in its entirety). In some embodiments, the immunotherapy of the present disclosure comprises targeting, antigen loading and activation of DCs in vivo, which results in vivo treatment of diseases by generating a beneficial immune response in a cancer patient.

In some embodiments, the DCs are generated in vivo or ex vivo from immature precursors (e.g., monocytes). For example, for ex vivo DC generation, a cell population enriched for DC precursor cells (e.g., peripheral blood mononuclear cells (PBMCs)) is obtained from a patient, and then the DC precursor cells are differentiated ex vivo into mature DCs. Typically, to generate immature dendritic cells (DC), one must first purify or enrich the monocytic precursors from other cell types. For example, peripheral blood mononuclear cells (PBMCs) are extracted from whole blood (e.g., over Ficoll density gradient centrifugation). Then the PBMCs will be used to generate monocytic dendritic cell precursors. In some embodiments, the DCs are generated from monocytes, CD34⁺ cells (i.e., cells expressing CD34), etc.

In certain embodiments, monocytic dendritic cell precursors are isolated by adherence to a monocyte-binding substrate. For example, a population of leukocytes (e.g., isolated by leukapheresis) is contacted with a monocytic dendritic cell precursor adhering substrate. When the population of leukocytes is contacted with the substrate, the monocytic dendritic cell precursors in the leukocyte population preferentially adhere to the substrate. In one embodiment, monocytes are isolated through adherence of the monocytic precursors to a plastic (polystyrene) surface, as the monocytes have a greater tendency to stick to plastic than other cells found in, for example, peripheral blood, such as lymphocytes and natural killer (NK) cells.

Methods for isolating cell populations enriched for dendritic cell precursors and immature dendritic cells from various sources, including blood and bone marrow, further include, in some embodiments, phlebotomy, apheresis or leukapheresis, collecting heparinized blood, preparing buffy coats, rosetting, centrifugation, density gradient centrifugation (e.g., using Ficoll, Percoll (colloidal silica particles of 15-30 mm diameter coated with polyvinylpyrrolidone (PVP)), sucrose, and the like), differential lysis of cells, filtration, and the like. In some embodiments, dendritic cell precursors can be selected using CD14 selection of G-CSF mobilized peripheral blood.

In some embodiments, before the subject's blood or bone marrow is obtained to isolate dendritic cell precursors, the subject is administered granulocyte macrophage colony stimulating factor (GM-CSF) to increase bone marrow production of monocytes and dendritic cell precursors. In certain embodiments, GM-CSF is administered at a dose ranging from about 10 μg/day to about 500 μg/day, from about 20 μg/day to about 300 μg/day, from about 50 μg/day to about 250 μg/day, from about 100 μg/day to about 300 μg/day, from about 200 μg/day to about 300 μg/day, about 200 μg/day, or about 250 μg/day. The dose of GM-CSF can also be lower or higher. In certain embodiments, GM-CSF may be administered for about 1 day, about 2 days, about 3 days, about 4 day, about 5 days, about 6 days, about 1 week, about 1.5 weeks, about 2 weeks, or longer. The dendritic cell precursors and/or immature dendritic cells are, in some embodiments, cultured and differentiated in suitable culture conditions. The tissue culture media is, for example, supplemented with, e.g., plasma, serum, amino acids, vitamins, cytokines (e.g., granulocyte-macrophage colony-stimulating factor (GM-CSF), interleukins such as Interleukin 4 (IL-4), Interleukin 13 (IL-13), Interleukin 15 (IL-15), or combinations thereof), purified proteins (such as serum albumin), divalent cations (e.g., calcium and/or magnesium ions), growth factors, and the like, to promote differentiation of the cells. In certain embodiments, the blood plasma or serum can be heat-inactivated. The plasma or serum can be autologous, allogeneic or heterologous to the cells. In certain embodiments, the dendritic cell precursors can be cultured in the serum-free media. In certain embodiments, such culture conditions optionally exclude any animal-derived products. In some embodiments, a dendritic cell culture medium contains about 200 units/ml to about 1500 units/ml (e.g., about 1000 units/ml, about 500 units/ml, etc.) of GM-CSF and about 200 units/ml to about 1500 units/ml (e.g., about 800 units/ml, about 500 units/ml, etc.) IL-4.

In some embodiments, the immunotherapy comprises administering mature dendritic cells to a cancer patient. In certain embodiments, such methods are performed by obtaining dendritic cell precursors or immature dendritic cells, differentiating and maturing those cells in the presence of a tumor-associated antigen or a tumor-associated peptide antigen (or a nucleic acid composition) to form a mature dendritic cell population. In some embodiments, the immature dendritic cells are contacted with antigen prior to or during maturation. The DC administration (vaccination) is, in certain embodiments, given once, twice, three times, four times, five times, six times, seven times, eight times, nine times, ten times, eleven times, twelve times, thirteen times, fourteen times, fifteen times, or more, within a treatment regime to a subject/patient. In some embodiments, the DC administration (vaccination) is given every 2 days, every 3 days, every 4 days, every 5 days, every 6 days, every 7 days, every 8 days, every 9 days, every 10 days, every 11 days, every 12 days, every 13 days, every 14 days, every 16 days, every 18 days, every 20 days, every 1 month, every 2 months, every 3 months, every 6 months, or at different frequencies.

In some embodiments, the DC is administered at a dose ranging from about 1×10³ DCs to about 1×10¹² DCs, from about 1×10⁴ DCs to about 1×10¹⁰ DCs, from about 1×10⁵ DCs to about 1×10⁹ DCs, from about 1×10⁶ DCs to about 1×10⁸ DCs, from about 1×10⁶ DCs to about 1×10⁷ DCs, from about 1×10⁷ DCs to about 1×10⁸ DCs, about 1×10⁵ DCs, about 1×10⁶ DCs, about 1×10⁷ DCs, about 1×10⁸ DCs, or about 1×10⁹ DCs. In a related embodiment, the mature dendritic cells are contacted with, and thus, activate, lymphocytes. The activated, polarized lymphocytes, optionally followed by clonal expansion in cell culture, are, in some instances, administered to a cancer patient, using the methods disclosed herein.

### Low Dose of a TNF-Alpha or LTβ Receptor Agonist

In certain embodiments, a method of treating patient having cancer comprises administering to the patient a low-dose of TNF-α or LTβ receptor agonist and an immunotherapy. In some embodiments, a cancer patient identified as having a reduced likelihood of responding to an immunotherapy, using methods as described herein, is administered a low-dose of TNF-α, wherein the TNF-α restores the sensitivity of said patient to the immunotherapy, for instance by inducing expression of MHC-I.

In various examples, a low dose of TNF-α comprises at least about 0.1 μg/m² to about 0.2 μg/m², about 0.15 μg/m² to about 0.25 μg/m², about 0.22 μg/m² to about 0.35 μg/m², about 0.3 μg/m² to about 0.4 μg/m², about 0.33 μg/m² to about 0.5 μg/m², about 0.4 μg/m²to about 0.6 μg/m², about 1 μg/m² to about 4 μg/m²,about 2 μg/m² to about 6 μg/m², about 4 μg/m² to about 8 μg/m², about 6 μg/m² to about 10 μg/m², about 8 μg/m² to about 15 μg/m², about 12 μg/m² to about 20 μg/m², about 15 μg/m² to about 25 μg/m², about 22 μg/m² to about 35 μg/m², about 30 μg/m² to about 40 μg/m². In some examples, the low dose of TNF-α comprises at least about 0.6 μg/m² to about 40 μg/m². The dosage of the ligand, in some embodiments, is about 5 fold to about 300 fold, or 10 fold to about 300 fold lower than the maximum tolerated dose in humans. The low dose of TNF-α, in some embodiments, is about 10 fold to about 50 fold, about 20 fold to about 80 fold, about 40 fold to about 100 fold, about 150 fold to about 200 fold, about 250 fold to about 300 fold lower than the maximum tolerated dose of TNF-α in humans. In some embodiments, LTβ receptor agonist is administered to restore the sensitivity of said patient to the immunotherapy, for instance by inducing expression of MHC-I. In various examples, a low dose of LTβ receptor agonist comprises at least about The dosage of the ligand, in some embodiments, is about 5 fold to about 300 fold, or 10 fold to about 300 fold lower than the maximum tolerated dose in humans. The low dose of LTβ receptor agonist, in some embodiments, is about 10 fold to about 50 fold, about 20 fold to about 80 fold, about 40 fold to about 100 fold, about 150 fold to about 200 fold, about 250 fold to about 300 fold lower than the maximum tolerated dose of LTβ receptor agonist in humans.

In some embodiments, additional molecules can restore the sensitivity of said patient to immunotherapy. In some embodiments, a method of treating a cancer patient, identified as having a reduced likelihood of responding to an immunotherapy, using methods as described herein, comprises administering a low-dose of another therapeutic agent, wherein the therapeutic agent restores the sensitivity of said patient to the immunotherapy. In some embodiments, the therapeutic agent comprises a ligand of TNFR1, TNFR2, 4-1BB, AITR, BCMA, CD27, CD40, Death receptor-3, Death receptor-6, Decoy receptor-3, EDAR, Fas, GITR, HVEM, LTβ-R, OPG, OX40, p75NGFR, RANK, TACI, TRAIL-R1, TRAIL-R2, TRAIL-R3, TRAIL-R4, Troy, or XEDAR. In some embodiments, the administered ligand comprises at least one of: Fas ligand, lymphotoxin, lymphotoxin alpha, lymphotoxin beta, 4-1BB Ligand, CD30 Ligand, EDA-A1, LIGHT, TLAI, TWEAK, or TRAIL.

In some embodiments, the immune checkpoint regulator used in immunotherapy comprises administering to the patient an immune checkpoint inhibitor or an immune checkpoint activator. In some embodiments, the immune checkpoint activator is an agoni st of co-stimulation by CD27, CD40, OX40, GITR, CD137, CD28, or ICOS. In some embodiments, the checkpoint activator is an agonist antibody that binds to CD27, CD40, OX40, GITR, CD137, CD28, or ICOS. In some embodiments, the immune checkpoint inhibitor of an antagonist of PD-1, PD-L1, CTLA-4, A2AR, B7-H3, BTLA, IDO, KIR, LAG3, TIM-3, VISTA, CD160, TIGIT, or PSGL-1. In some embodiments, the immune checkpoint inhibitor is an antagonist antibody that binds to PD-1, PD-L1, CTLA-4, A2AR, B7-H3, BTLA, IDO, KIR, LAG3, TIM-3, VISTA, CD160, TIGIT, or PSGL-1 In some embodiments, the cancer comprises a solid, tumor, lymphoma, or leukemia. In some embodiments, the cancer comprises medulloblastoma. In some embodiments, the method comprising administering a low dose of TNF-α and an anti-PD-1 antibody is used. In some embodiments, the method comprising administrating LTβ receptor agonist and an anti-PD-1 antibody is implemented.

In some embodiments, the low dose of TNF-α or LTβ receptor agonist is administered to a cancer patient after said patient has been identified as having reduced likelihood of responding to an immunotherapy due to presence of a loss-of-function TP53 mutation in a biological sample isolated from the patient, using any of the methods as described herein. In some embodiments, the TNF-α or LTβ receptor agonist is co-administered with the immunotherapy. In some embodiments, the TNF-α or LTβ receptor agonist is administered prior to the immunotherapy. The immunotherapy is administered, in some embodiments, in a treatment regimen comprising multiple doses. In some examples, the immunotherapy is administered in a treatment regimen comprising multiple doses such that not every dose is preceded by or co-administered with a low dose of TNF-α or LTβ receptor agonist. In some examples, the immunotherapy is administered in a treatment regimen comprising multiple doses such that every new dose of immunotherapy is preceded by or co-administered with a low dose of TNF-α or LTβ receptor agonist. In some examples, the immunotherapy is administered in a treatment regimen comprising multiple doses such that every new dose of immunotherapy is preceded by or co-administered with a low dose of TNF-α or LTβ receptor agonist. In some example, the immunotherapy is administered in a treatment regimen comprising multiple doses such that every dose of immunotherapy is preceded by or co-administered with a low-dose of TNF-α or LTβ receptor agonist, unless TNF-α or LTβ receptor agonist was administered within 1 day, 2 day, 3 day, 7 day, or 14 days of the immunotherapy dose. In other examples, the immunotherapy is administered in a treatment regimen comprising multiple doses such that every other dose of immunotherapy is preceded by or co-administered with a low dose of TNF-α or LTβ receptor agonist. In other examples, the immunotherapy is administered in a treatment regimen comprising multiple doses such that every third, fourth, fifth, sixth, seventh, eighth, ninth, or tenth dose of immunotherapy is preceded by or co-administered with a low dose of TNF-α or LTβ receptor agonist. The low dose of TNF-α or LTβ receptor agonist, in some cases, is administered about 7 days, about 3 days, about 2 days, about 1 day, about 12 hours, about 6 hours prior to a dose of the immunotherapy.

In some embodiments, the TNF-α or LTβ receptor agonist is administered at any suitable frequency, such as, for example, frequency of once a day, every other day, twice weekly, once weekly, once every 2 weeks, once every 3 weeks or once every 4 weeks; and the immunotherapy is administered at the same frequency as the TNF-α or LTβ receptor agonist or at a different frequency, wherein each administration of the immunotherapy is preceded by an administration of TNF-α or LTβ receptor agonist by about 7 days, about 3 days, about 2 days, about 1 day, about 12 hours, about 6 hours. For example, in some instance, the immunotherapy is administered twice weekly, once weekly, once every 2 weeks, once every 3 weeks, once every 4 weeks, once every 6 weeks, once every 2 months, once every 3 months, once every 4 months, once every 5 months, or once every 6 months; wherein each administration of the immunotherapy is preceded by an administration of TNF-α or LTβ receptor agonist by about 7 days, about 3 days, about 2 days, about 1 day, about 12 hours, about 6 hours.

An exemplary dosage regimen comprises administration of the TNF-α or LTβ receptor agonist twice weekly, while the immunotherapy is administered once a week, where each administration of the immunotherapy is preceded by an administration of TNF-α or LTβ receptor agonist by not more than 2 days. For example, the immunotherapy is administered, in some instances, once every three weeks or once every four weeks, while the TNF-α or LTβ receptor agonist is administered twice weekly. In some examples, each administration of the immunotherapy is preceded by an administration of the TNF-α or LTβ receptor agonist by not more than 7 days. In some examples, each administration of the immunotherapy is preceded by an administration of the TNF-α or LTβ receptor agonist by not more than 3 days. In other examples, the TNF-α or LTβ receptor agonist is administered twice weekly and the immunotherapy is administered twice weekly, wherein each administration of the immunotherapy is preceded by an administration of TNF-α or LTβ receptor agonist by not more than 2 days. In some examples, each administration of the immunotherapy is preceded by an administration of the TNF-α or LTβ receptor agonist by not more than 1 day. In some examples, each administration of the immunotherapy is preceded by an administration of the TNF-α or LTβ receptor agonist by not more than 12 hours. In some examples, each administration of the immune checkpoint inhibitor is preceded by an administration of the TNF-α or LTβ receptor agonist by not more than 6 hours.

In some embodiments, administering a low-dose of TNF-α or LTβ receptor agonist, as described above, is followed by administration of an immunotherapy, such as an immune checkpoint therapy, an adoptive T cell therapy, a dendritic cell vaccination, or any combinations thereof. In some embodiments, the cancer patient administered with a low-dose of TNF-α or LTβ receptor agonist demonstrates increased likelihood of responding to an immunotherapy, wherein the increased likelihood of response is measured using any of the methods discussed above. In some embodiments, the administration of certain ligands can be implemented as exemplified above to restore sensitivity to immunotherapy. In some embodiments, this is done using the same method described above in reference to TNF-α and LTβ receptor agonist. These ligands can bind to one or more proteins comprising TNFR1, TNFR2, 4-1BB, AITR, BCMA, CD27, CD40, Death receptor-3, Death receptor-6, Decoy receptor-3, EDAR, Fas, GITR, HVEM, LTβ-R, OPG, OX40, p75NGFR, RANK, TACI, TRAIL-R1, TRAIL-R2, TRAIL-R3, TRAIL-R4, Troy, or XEDAR. In some embodiments, the administered ligand comprises at least one of: Fas ligand, lymphotoxin, lymphotoxin alpha, lymphotoxin beta, 4-1BB Ligand, CD30 Ligand, EDA-A1, LIGHT, TLAI, TWEAK, and TRAIL.

### Pharmaceutical Compositions

Pharmaceutical compositions containing an agent for immunotherapy methods described above, or TNF-α, LTβ receptor agonist, another therapeutic agent, or any combinations thereof, are provided in some embodiments of this disclosure. In some embodiments, the pharmaceutical composition comprises TNF-α. In some embodiments, the pharmaceutical composition comprises an LTβ receptor agonist. In some embodiments, the pharmaceutical compositions of this disclosure are prepared as solutions, dispersions in glycerol, liquid polyethylene glycols, and any combinations thereof in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof. In some embodiments, a pharmaceutical composition as described herein comprises an excipient. An excipient is, in some examples, an excipient described in the Handbook of Pharmaceutical Excipients, American Pharmaceutical Association (1986). Non-limiting examples of suitable excipients include a buffering agent, a preservative, a stabilizer, a binder, a compaction agent, a lubricant, a chelator, a dispersion enhancer, a disintegration agent, a flavoring agent, a sweetener, a coloring agent.

In some embodiments an excipient is a buffering agent. Non-limiting examples of suitable buffering agents include histidine, sodium citrate, magnesium carbonate, magnesium bicarbonate, calcium carbonate, and calcium bicarbonate. As a buffering agent, histidine, sodium bicarbonate, potassium bicarbonate, magnesium hydroxide, magnesium lactate, magnesium glucomate, aluminium hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof is used, in some embodiments, in a pharmaceutical composition of the present disclosure.

In some embodiments an excipient comprises a preservative. Non-limiting examples of suitable preservatives include antioxidants, such as alpha-tocopherol and ascorbate, and antimicrobials, such as parabens, chlorobutanol, and phenol. In some examples, antioxidants further include but are not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments a pharmaceutical composition as described herein comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinyl alcohols, C₁₂-C₁₈ fatty acid alcohol, polyethylene glycol, polyols, saccharides, oligosaccharides, and combinations thereof. The binders used in a pharmaceutical formulation are, in some examples, selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or any combinations thereof.

In some embodiments a pharmaceutical composition as described herein comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that are used in a pharmaceutical formulation, in some embodiments, are be selected from metallic stearates (such as magnesium stearate, calcium stearate, aluminium stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments a pharmaceutical formulation comprises a dispersion enhancer as an excipient. Non-limiting examples of suitable dispersants include, in some examples, starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments a pharmaceutical composition as described herein comprises a disintegrant as an excipient. In some embodiments a disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments a disintegrant is an effervescent disintegrant. Non-limiting examples of suitable effervescent disintegrants include sodium bicarbonate in combination with citric acid, and sodium bicarbonate in combination with tartaric acid.

In some embodiments an excipient comprises a flavoring agent. Flavoring agents incorporated into an outer layer are, in some examples, chosen from synthetic flavor oils and flavoring aromatics; natural oils; extracts from plants, leaves, flowers, and fruits; and combinations thereof In some embodiments a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

In some instances, a pharmaceutical composition as described herein comprises a coloring agent. Non-limiting examples of suitable color agents include food, drug and cosmetic colors (FD&C), drug and cosmetic colors (D&C), and external drug and cosmetic colors (Ext. D&C). A coloring agents can be used as dyes or their corresponding lakes.

In some instances, a pharmaceutical composition as described herein comprises a chelator. In some cases, a chelator is a fungicidal chelator. Examples include, but are not limited to: ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-tetraacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the tri sodium salt of nitrilotris(methylenephosphoric acid); 7,19,30-trioxa-1,4,10,13,16,22,27,33-octaazabicyclo[11,11,11] pentatriacontane hexahydrobromide; or triethylenetetramine-N,N,N′,N″,N′“,N′”-hexaacetic acid.

Also contemplated are combination products that include one or more immunotherapeutic agents disclosed herein and one or more other antimicrobial or antifungal agents, for example, polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine or other antifungal agents, including those described herein. In addition, it is contemplated that a peptide can be combined with topical antifungal agents such as ciclopirox olamine, haloprogin, tolnaftate, undecylenate, topical nysatin, amorolfine, butenafine, naftifine, terbinafine, and other topical agents. In some instances, a pharmaceutical composition comprises an additional agent. In some cases, an additional agent is present in a therapeutically effective amount in a pharmaceutical composition.

Under ordinary conditions of storage and use, the pharmaceutical compositions as described herein comprise a preservative to prevent the growth of microorganisms. In certain examples, the pharmaceutical compositions as described herein do not comprise a preservative. The pharmaceutical forms suitable for injectable use include sterile aqueous solutions or dispersions and sterile powders for the extemporaneous preparation of sterile injectable solutions or dispersions. The pharmaceutical compositions comprise a carrier which is a solvent or a dispersion medium containing, for example, water, ethanol, polyol (e.g., glycerol, propylene glycol, and liquid polyethylene glycol, and the like), and/or vegetable oils, or any combinations thereof. Proper fluidity is maintained, for example, by the use of a coating, such as lecithin, by the maintenance of the required particle size in the case of dispersion and by the use of surfactants. The prevention of the action of microorganisms is brought about by various antibacterial and antifungal agents, for example, parabens, chlorobutanol, phenol, sorbic acid, thimerosal, and the like. In many cases, isotonic agents are included, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the liquid dosage form is suitably buffered if necessary and the liquid diluent rendered isotonic with sufficient saline or glucose. The liquid dosage forms are especially suitable for intravenous, intramuscular, subcutaneous, intratumoral, and intraperitoneal administration. In this connection, sterile aqueous media that can be employed will be known to those of skill in the art in light of the present disclosure. For example, one dosage is dissolved, in certain cases, in 1 mL to 20 mL of isotonic NaCl solution and either added to 100 mL to 1000 mL of a fluid, e.g., sodium-bicarbonate buffered saline, or injected at the proposed site of infusion.

In certain embodiments, sterile injectable solutions is prepared by incorporating a immunotherapy agent, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. The compositions disclosed herein are, in some instances, formulated in a neutral or salt form. Pharmaceutically-acceptable salts include, for example, the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups are, in some cases, derived from inorganic bases such as, for example, sodium, potassium, ammonium, calcium, or ferric hydroxides, and such organic bases as isopropylamine, trimethylamine, histidine, procaine and the like. Upon formulation, the pharmaceutical compositions are administered, in some embodiments, in a manner compatible with the dosage formulation and in such amount as is therapeutically effective.

In certain embodiments, a pharmaceutical composition of this disclosure comprises an effective amount of an immunotherapy agent, as disclosed herein, combined with a pharmaceutically acceptable carrier. “Pharmaceutically acceptable,” as used herein, includes any carrier which does not interfere with the effectiveness of the biological activity of the active ingredients and/or that is not toxic to the patient to whom it is administered. Non-limiting examples of suitable pharmaceutical carriers include phosphate buffered saline solutions, water, emulsions, such as oil/water emulsions, various types of wetting agents and sterile solutions. Additional non-limiting examples of pharmaceutically compatible carriers can include gels, bioadsorbable matrix materials, implantation elements containing the immunotherapeutic agents or any other suitable vehicle, delivery or dispensing means or material. Such carriers are formulated, for example, by conventional methods and administered to the subject at an effective amount.

In some embodiments, the pharmaceutical composition is a formulation comprising an immunotherapy agent (e.g., an immune check point inhibitor, regulator, or activator) and a buffering agent. In some embodiments, the immunotherapy agent is present at a concentration of about 10 to about 50 mg/mL, about 15 to about 50 mg/mL, about 20 to about 45 mg/mL, about 25 to about 40 mg/mL, about 30 to about 35 mg/mL, about 25 to about 35 mg/mL, or about 30 to about 40 mg/mL, about 15 mg/mL, about 20 mg/mL, about 25 mg/mL, about 30 mg/mL, about 33.3 mg/mL, about 35 mg/mL, about 40 mg/mL, about 45 mg/mL, or about 50 mg/mL. In some embodiments, the formulation comprises a buffering agent comprising histidine (e.g., a histidine buffer). In certain embodiments, the buffering agent (e.g., histidine buffer) is present at a concentration of about 1 mM to about 20 mM, about 2 mM to about 15 mM, about 3 mM to about 10 mM, about 4 mM to about 9 mM, about 5 mM to about 8 mM, or about 6 mM to about 7 mM, about 1 mM, about 2 mM, about 3 mM, about 4 mM, about 5 mM, about 6 mM, about 6.7 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM. In some embodiments, the buffering agent (e.g., histidine buffer) is present at a concentration of about 6 mM to about 7 mM, about 6.7 mM. In other embodiments, the buffering agent (e.g., a histidine buffer) has a pH of about 4 to about 7, about 5 to about 6, about 5.5, or about 6.

In some embodiments, the formulation further comprises a carbohydrate. In certain embodiments, the carbohydrate is sucrose. In some embodiments, the carbohydrate (e.g., sucrose) is present at a concentration of about 50 mM to about 150 mM, about 25 mM to about 150 mM, about 50 mM to about 100 mM, about 60 mM to about 90 mM, about 70 mM to about 80 mM, or about 70 mM to about 75 mM, about 25 mM, about 50 mM, about 60 mM, about 70 mM, about 80 mM, about 90 mM, about 100 mM, or about 150 mM.

In some embodiments, the formulation further comprises a surfactant. In certain embodiments, the surfactant is polysorbate 20. In some embodiments, the surfactant or polysorbate 20) is present at a concentration of about 0.005% to about 0.025% (w/w), about 0.0075% to about 0.02% or about 0.01% to 0.015% (w/w), about 0.005%, about 0.0075%, about 0.01%, about 0.013%, about 0.015%, or about 0.02% (w/w). In certain embodiments, the formulation is a reconstituted formulation. For example, a reconstituted formulation is prepared, in some instances, by dissolving a lyophilized formulation in a diluent such that the immunotherapy agent is dispersed in the reconstituted formulation. In some embodiments, the lyophilized formulation is reconstituted with about 0.5 mL to about 2 mL, such as about 1 mL, of water or buffer for injection. In certain embodiments, the lyophilized formulation is reconstituted with 1 mL of water for injection at a clinical site.

### Combination Therapies

In certain embodiments, the methods of this disclosure comprise administering an immunotherapy as disclosed herein, followed by, and preceded by or in combination with one or more further therapy. Examples of the further therapy can include, but are not limited to, chemotherapy, radiation, an anti-cancer agent, or any combinations thereof. The further therapy can be administered concurrently or sequentially with respect to administration of the immunotherapy. In certain embodiments, the methods of this disclosure comprise administering an immunotherapy as disclosed herein, followed by, preceded by, or in combination with one or more anti-cancer agents or cancer therapies. Anti-cancer agents include, but are not limited to, chemotherapeutic agents, radiotherapeutic agents, cytokines, immune checkpoint inhibitors, anti-angiogenic agents, apoptosis-inducing agents, anti-cancer antibodies and/or anti-cyclin-dependent kinase agents. In certain embodiments, the cancer therapies include chemotherapy, biological therapy, radiotherapy, immunotherapy, hormone therapy, anti-vascular therapy, cryotherapy, toxin therapy and/or surgery or combinations thereof. In certain embodiments, the methods of this disclosure include administering an immunotherapy, as disclosed herein, followed by, preceded by or in combination with one or more further immunomodulatory agents. An immunomodulatory agent includes, in some examples, any compound, molecule or substance capable of suppressing antiviral immunity associated with a tumor or cancer. Non-limiting examples of the further immunomodulatory agents include anti-CD33 antibody or variable region thereof, an anti-CD11b antibody or variable region thereof, a COX2 inhibitor, e.g., celecoxib, cytokines, such as IL-12, GM-CSF, IL-2, IFN3 and 1FNy, and chemokines, such as MIP-1, MCP-1 and IL-8.

In certain examples, where the further therapy is radiation exemplary doses are 5,000 Rads (50 Gy) to 100,000 Rads (1000 Gy), or 50,000 Rads (500 Gy), or other appropriate doses within the recited ranges. Alternatively, the radiation dose are about 30 to 60 Gy, about 40 to about 50 Gy, about 40 to 48 Gy, or about 44 Gy, or other appropriate doses within the recited ranges, with the dose determined, example, by means of a dosimetry study as described above. “Gy” as used herein can refer to a unit for a specific absorbed dose of radiation equal to 100 Rads. Gy is the abbreviation for “Gray.”

In certain examples, where the further therapy is chemotherapy, exemplary chemotherapeutic agents include without limitation alkylating agents (e.g., nitrogen mustard derivatives, ethylenimines, alkylsulfonates, hydrazines and triazines, nitrosureas, and metal salts), plant alkaloids (e.g., vinca alkaloids, taxanes, podophyllotoxins, and camptothecan analogs), antitumor antibiotics (e.g., anthracyclines, chromomycins, and the like), antimetabolites (e.g., folic acid antagonists, pyrimidine antagonists, purine antagonists, and adenosine deaminase inhibitors), topoisomerase I inhibitors, topoisomerase II inhibitors, and miscellaneous antineoplastics (e.g., ribonucleotide reductase inhibitors, adrenocortical steroid inhibitors, enzymes, antimicrotubule agents, and retinoids). Exemplary chemotherapeutic agents can include, without limitation, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), Ibrutinib, idelalisib, and brentuximab vedotin.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary anthracyclines can include, without limitation, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, but are not limited to, vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors can, but are not limited to, bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoac etamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

“In combination with,” as used herein, means that the immunotherapy and the further therapy are administered to a subject as part of a treatment regimen or plan. In certain embodiments, being used in combination does not require that the immunotherapy and the further therapy are physically combined prior to administration or that they be administered over the same time frame. For example, and not by way of limitation, the immunotherapy and the one or more agents are administered concurrently to the subject being treated, or are administered at the same time or sequentially in any order or at different points in time.

The further therapy is administered, in various embodiments, in a liquid dosage form, a solid dosage form, a suppository, an inhalable dosage form, an intranasal dosage form, in a liposomal formulation, a dosage form comprising nanoparticles, a dosage form comprising microparticles, a polymeric dosage form, or any combinations thereof. In certain embodiments, the further therapy is administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the further therapy is, in certain instances, once daily, twice daily, once every week, once every three weeks, once every four weeks (or once a month), once every 8 weeks (or once every 2 months), once every 12 weeks (or once every 3 months), or once every 24 weeks (once every 6 months).

### Cancer Targets

In an embodiment of this disclosure, a method of treatment for a hyperproliferative disease, such as a cancer or a tumor, by administering an immunotherapy to a cancer patient only if said patient does not comprise a loss-of-function TP53 mutation, is contemplated. Cancers that can be treated include, but are not limited to, medulloblastoma, melanoma, hepatocellular carcinoma, breast cancer, lung cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, and sarcoma. Cancer cells that can be treated by the methods of this disclosure include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydi g cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### EXAMPLES

The examples below further illustrate the described embodiments without limiting the scope of the disclosure.

### EXAMPLE 1

### TP53 Regulates Class 1 MHC Molecules in Medulloblastoma Tumor Cells

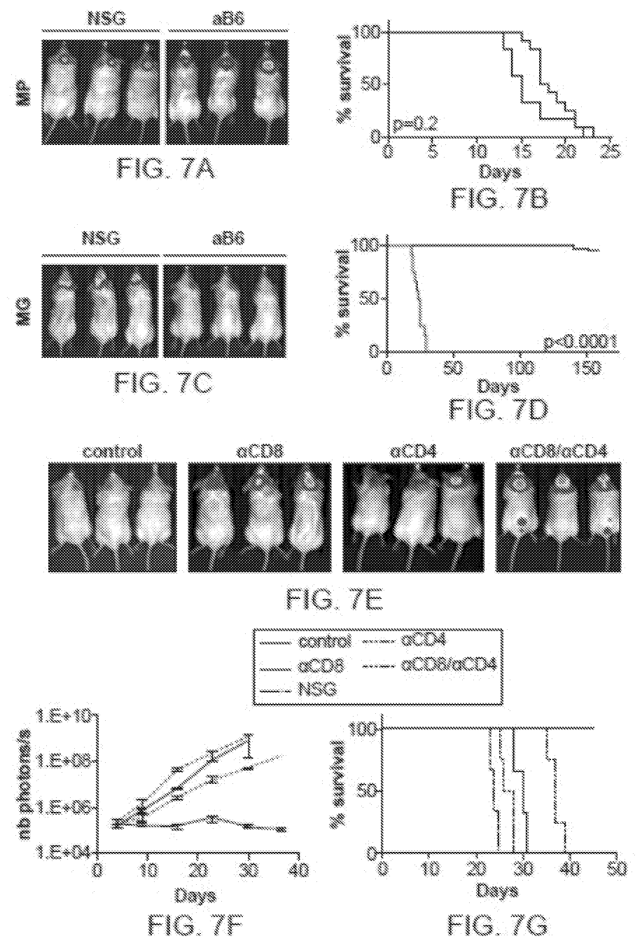

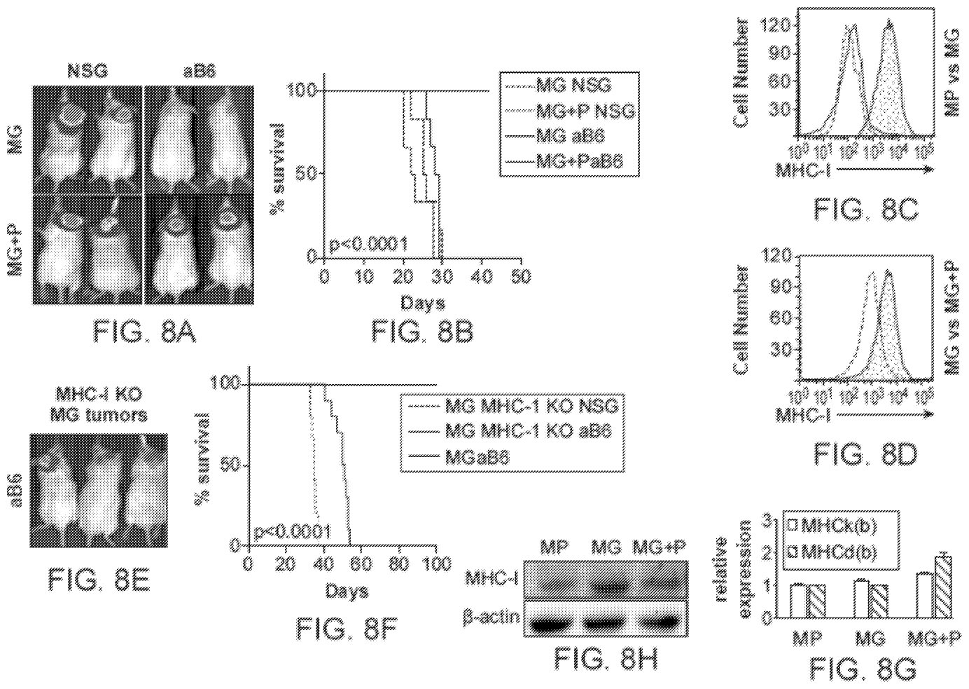

Genetically engineered mouse models of MYC-driven medulloblastoma, also known as Group 3 MB, were created by infecting cerebellar stem cells with viruses encoding either (a) Myc and a dominant negative form of TP53 (MP) or (b) Myc and the transcriptional repressor Gfi1 (MG), and transplanting suitable quantities of the infected stem cells into the cerebellum of immunodeficient (NOD-SCID-IL2Rgamma knockout; hereafter referred to as NSG) mice. Within 6-10 weeks, 100% of the mice developed aggressive tumors that resembled human Group 3 MB at a histological and molecular level. Immunocompetent (albino C57BL/6; hereafter referred to as aB6) mice were also transplanted with the stem cells, as specified above, with the MP or the MG cells. As shown in FIG. 2, it was observed that the MP tumors grew in either NSG or aB6 mice (FIG. 2A). While the MG tumors grew in NSG mice (FIG. 2B), only 4.4% of the aB6 mice transplanted with MG tumor cells went onto develop tumors, albeit with much longer latency than those in NSG mice (FIGS. 7C-D).

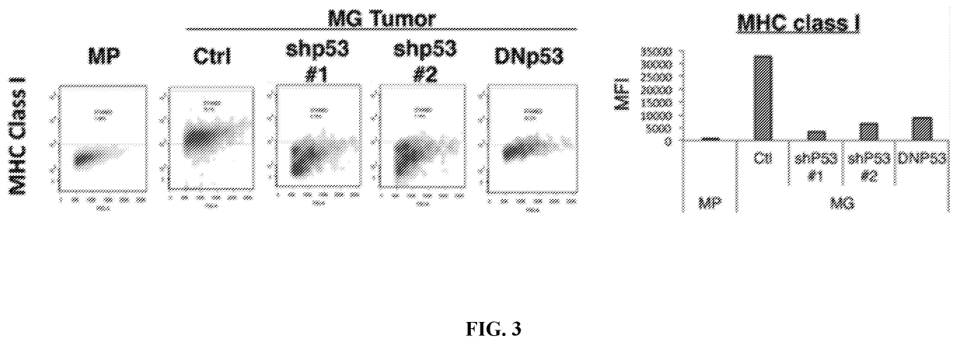

The MP and MG types of the medulloblastoma tumor cells were analyzed to assay the expression of MHC-I on their surfaces. As shown in FIG. 3, the MP tumor cells, which were derived from cells infected with viruses encoding Myc and a dominant negative form of TP53 (also referred to herein as DNp53), expressed lower levels of MHC-I on their surface compared to MG tumor cells. MHC-I levels were drastically reduced when TP53 was inhibited in the MG cells with shRNA or by expressing DNp53, also shown in FIG. 3, and tumor cells were no longer rejected in aB6 mice and tumors were able to form. The above finding suggested that TP53 regulates expression of MHC-I molecules in tumor cells, as shown in FIG. 1.

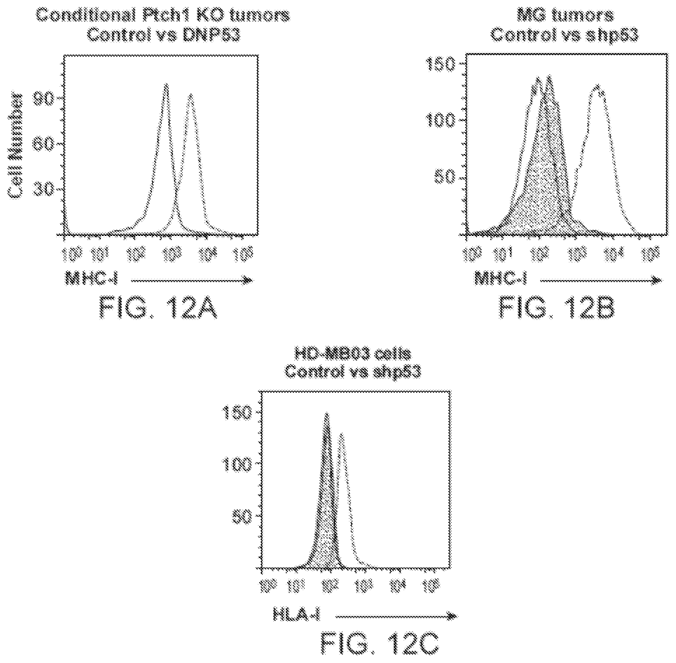

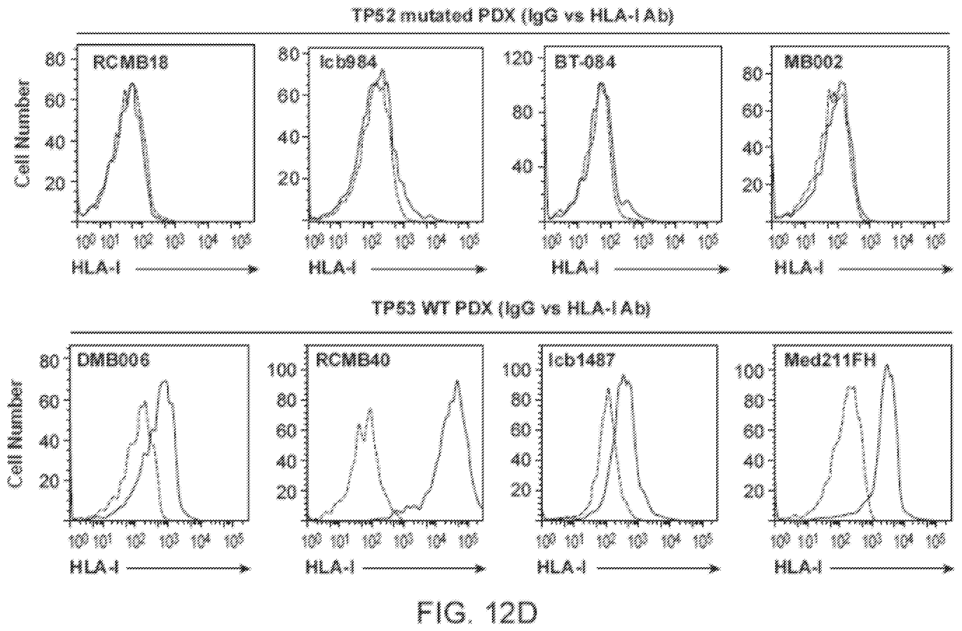

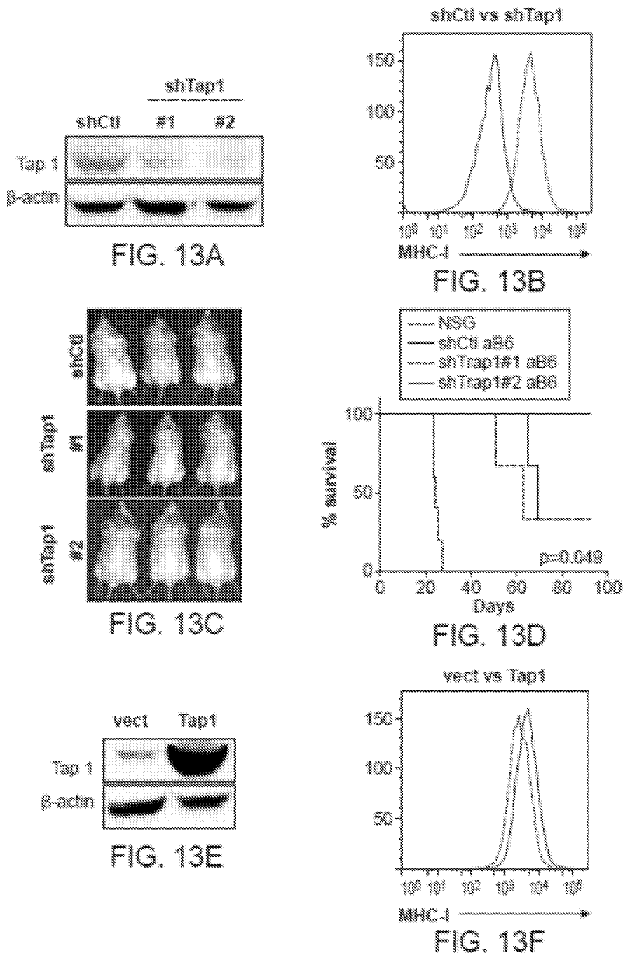

Moreover, tumor cells from conditional Ptch 1 knockout (Math1-CreER^{T2}; Ptch1^{flxo/flox}) mice, a model for SHH-driven medulloblastoma, were implanted into NSG mice. When the animal showed signs of medulloblastoma, the tumors were harvested and re-suspended in media. Tumors showed downregulation of MHC-I following overexpression of DNp53 (FIG. 12A). Similarly, decreased expression of MHC-I was also seen following the shRNA-mediated knockdown of TP53 in MG tumor cells and TP53 in the human Group 3 MB cell line HD-MB03 (FIG. 12C). Finally, medulloblastoma patient-derived xenografts (PDXs) with TP53 mutations showed significantly less MHC-I (HLA-I) than PDXs with wild type TP53 (FIG. 12D). The above finding suggested that TP53 regulated expression of MHC-I molecules in tumor cells is necessary for expression of cell surface MHC-I.

### EXAMPLE 2

### Loss of MHC-I is Sufficient to Allow Tumors to Escape Immune Attack

This study was directed at determining if the lack of MHC-I is sufficient to render MG tumors capable of growing in immunocompetent mice. MG tumors were generated from mice lacking MHC-I (MHC-I knockout) and transplanted into NSG and aB6 mice. The mice were analyzed using bioluminescence imaging. If the growth of tumors increases, this would suggest that the lack of MHC-I expression may render MG tumors capable of growing in immunocompetent mice.

The MHC-I knockout MG tumor cells were able to grow in aB6 mice (FIGS. 8E-F). The loss of MHC-I in MG cells was sufficient to allow tumors to escape immune attack. The above findings suggested that MP tumors ability to grow in immunocompetent mice may be due to the reduced expression of MHC-I molecules.

### EXAMPLE 3

### TP53 Mutation Regulates MHC-I Levels in Pancreatic Cancer

This study was directed at determining whether TP53 mutation is correlated to expression levels of MHC-I. Tumor tissue was dissociated into a cell suspension and tumor cells and blood cells from the same patient were stained with fluorescently labeled antibodies specific for MHC-I (e.g., clone W6/32 from BD Biosciences). Tumor cells and blood cells were analyzed by flow cytometry to determine levels of MHC-I. If tumor cells have significantly less MHC-I on their surface, this would suggest that the tumor has an increased likelihood of being resistant to immunotherapy.

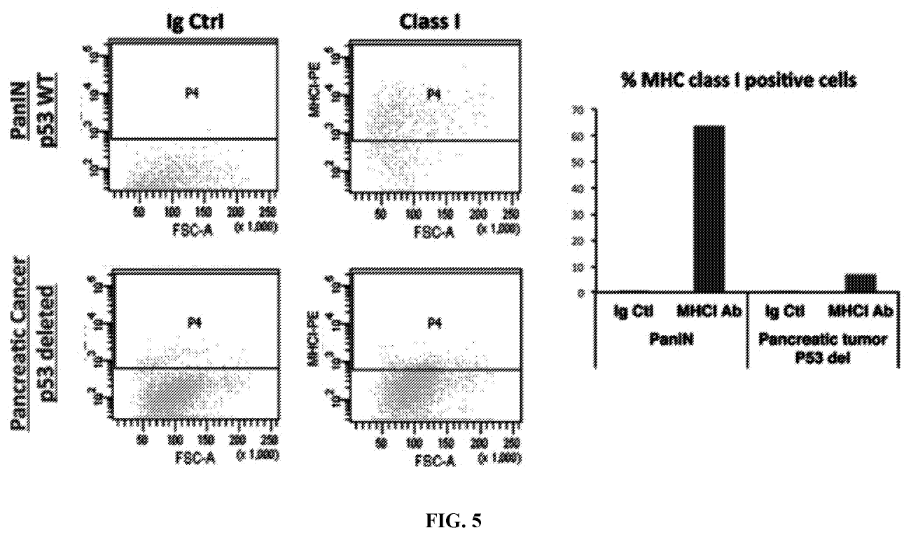

Pancreatic cells were isolated from transgenic mice overexpressing activated Kras alone (Ptfla-Cre; Kras^{LSL-G12D/+}) or activated Kras in conjunction with loss of TP53 (Ptfla-Cre; Kras^{LSL-G12D/+}; TP53^{f/f}). Cells were stained with fluorescent antibodies specific for MHC-I and analyzed by flow cytometry. As shown in FIG. 5, cells carrying mutations in TP53 (the mouse homolog of TP53) had significantly reduced levels of MHC-I compared to cells that contained wild-type TP53. The results suggested that tumors harboring TP53 mutations had diminished MHC-I on their surfaces and a reduced likelihood of responding to immunotherapy.

### EXAMPLE 4

### T Cells Inhibit Medulloblastoma Tumor Growth

This study was directed at determining whether the failure of MG tumors to grow in immunocompetent mice is mediated by the immune system. Genetically engineered mouse models of Group 3 MB were created by infecting cerebellar stem cells with viruses encoding Myc and Gfi1 (to generate MG tumors) and transplanting suitable quantities of the infected stem cells into the cerebellum of aB6 mice. The hosts were injected with antibodies to deplete CD4+ (helper) or CD8+ (cytotoxic) T cells. The growth of MG medulloblastoma tumor cells was analyzed using bioluminescence imaging. If the growth of tumors significantly increases, this would suggest that MG tumors are unable to grow in immunocompetent mice due to rejection by T cells.

Depletion of T cells resulted in increased tumor growth in aB6 mice, and the depletion of both T cell types resulted in slightly faster growth rates than the depletion of either cell type alone (FIGS. 7E-G). The above findings suggested that the failure of MG tumor growth in immunocompetent mice is due to rejection by T cells.

### EXAMPLE 5

### Expression of DNp53 Renders Tumor Cells Resistant to Rejection

This study was directed to determining whether the type of tumor, namely MP and MG, can alter T cell activation. MP tumors were transduced with Gfi1 (MP+G), and MG tumors were transduced with DNp53 (MG+P). The modified tumor cells were transplanted into NSG and aB6 mice. The mice were analyzed using bioluminescence imaging. If the transduced tumors showed increased growth, this would suggest that there was a decreased tendency of T cell activation.

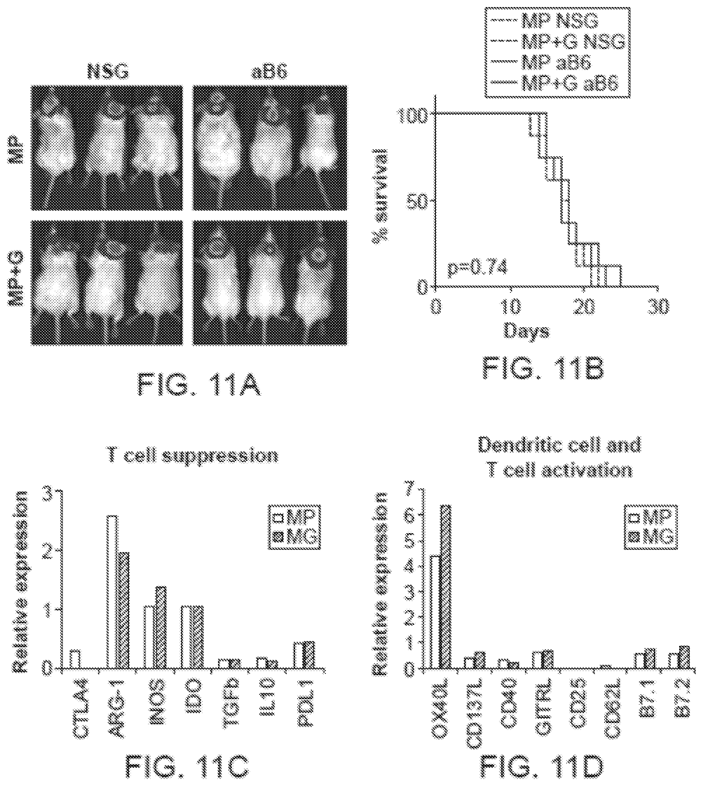

As shown in FIGS. 11A-B, the expression of Gfi1 had no effect on the growth of MP tumors. In MG tumor cells, the overexpression of DNp53 had a dramatic effect on the MG tumor, resulting in tumor growth in immunocompetent mice (FIGS. 8A-B). The above findings suggested that DNp53 renders tumor cells resistant to the rejection by T cells.

To further determine the impact of DNp53 resistance, the MP and MG tumors were analyzed for the expression of molecules known to regulate immune responses. RNA was prepared from the transplanted cells and subjected to quantitative RT-PCR using primers specific for each molecule. If the expression of these molecules varied, this would suggest that such molecules impact the resistant of the tumor cell to rejection.

The results displayed no differences in the expression of molecules that have been reported to regulate T cell responses, including cytotoxic T-lymphocyte associated protein 4 (CTLA-4), Arginase 1 (ARG-1), inducible nitric oxide synthase (iNOS), indoleamine 2,3-dioxygenase (IDO), transforming growth factor beta (TGFβ), interleukin-10 (IL-10), or programmed cell death ligand 1 (PD-L1) (FIG. 11C). Molecules that regulate activation of T cells and dendritic cells, including OX40 ligand (OX40L), CD137 ligand (CD137L), CD40, Glucocorticoid-Induced TNF-Related Ligand (GITRL), CD25, CD62 ligand (CD62L), B Lymphocyte activation antigen B7-1 (CD80) and B lymphocyte activation antigen B7-2 (CD86) were also not differentially expressed (FIG. 11D). The above findings suggested that the expression of molecules known to regulate immune responses does not impact resistance of the tumor cell to rejection.

### EXAMPLE 6

### TP53 Regulates the Cell Surface Localization of MHC-I

This study was directed to determining the impact of Tap1 and Erap1 cells in the expression of MHC-I. To analyze mRNA levels, tumor cells were subjected to quantitative RT-PCR. To analyze protein levels, western blotting was implemented.

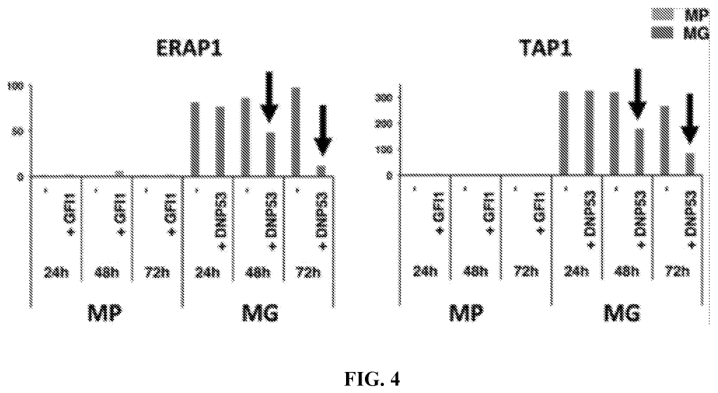

It was observed that TP53 regulated TAP1 and ERAP1 molecules in both MP and MG type medulloblastoma tumor cells as well as in the human MB cell line HDMB03. FIG. 4 shows that loss of TP53 inhibits RNA expression of TAP1 and ERAP1. While MP tumors have markedly decreased cell surface MHC-I, the levels of MHC-I mRNA and total cellular MHC-I protein were not different from MG tumors (FIGS. 8G-H). These finding suggested that TP53 is not regulating the expression of MHC-I, but rather, its cell surface localization.

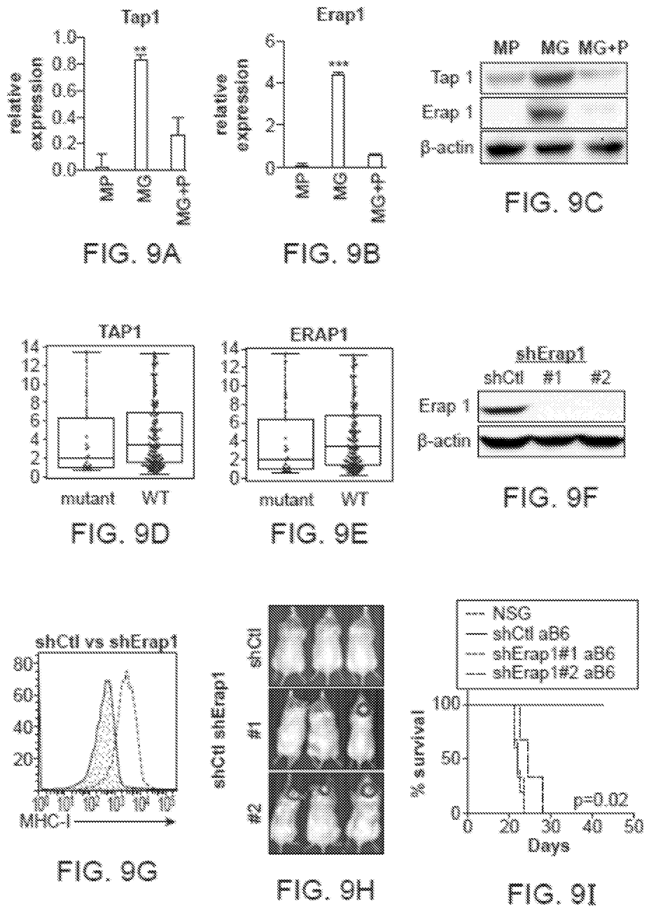

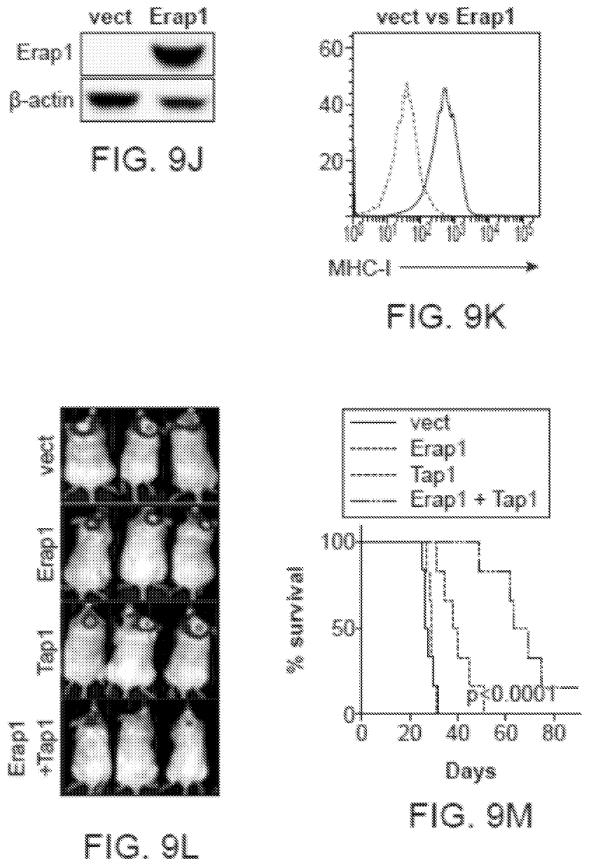

Moreover, the surface localization of MHC-I requires at least two proteins, Tap1 and Erap 1. Both proteins are reported targets of p53. To analyze Tap1 and Erap1 expression, protein and mRNA levels were measured. As shown in FIGS. 9A-C, MP tumors express significantly less Tap1 and Erap1 than MG tumors at both the protein and mRNA levels. In addition, transduction of MG tumors with DNp53 resulted in a significant downregulation of Tap1 and Erap1. Similarly, analysis of TAP1 and ERAP1 expression in human Group 3 MB cell line HD-MB03 samples revealed that tumors with TP53 mutations have lower levels of these genes than tumors with wild type 3 (FIGS. 9D-E). These results suggested that the lack of MHC-I cell surface localization in MP tumors may be due to TP53 regulation of Tap1 and Erap1 in both murine and human medulloblastoma.

### EXAMPLE 7

### Erap1 and Tap1 Contributes to the Resistance of p53-Mutant Tumors to Immune Rejection

This study was directed to determining if the resistance to T cell attack was caused by the downregulation of Tap1 and Erap1. MG tumor cells were transduced with control shRNA (shCtl) or shRNAs targeting Erap1 (shErap1#1, shErap1#2) and transplanted into NSG or aB6 mice. The same process was followed with shCtl or shRNAs targeting Tap1 (shTap1#1, shErap1#2). Knockdown efficiency was determined by western blotting. MHC-I expression was determined by fluorescence activated cell counting (FACS) in control cells and knockdown cells. Bioluminescence imaging measured the different in survival rates. If downregulation of these genes results in effects similar to the loss of TP53, this would suggest that the loss of Tap1 and Erap1 causes the resistance of MP tumor cells to a T cell attack.

As shown in FIGS. 4 and 9A-C, shRNA-mediated knockdown of Erap1 in MG tumor cells caused a significant decrease in MHC-I expression and resulted in the growth of MP tumor cells. The knockdown of Tap1 also deceased MHC-I expression and allowed some tumor growth, albeit with markedly prolonged latency compared to NSG mice.

In addition, the results of overexpression of Erap1 and Tap1 were analyzed. MP tumor cells were transduced with empty vectors or vectors encoding Erap1, Tap 1, or both and transplanted into NSG or aB6 mice. Efficiency of overexpression was determined by western blotting, and the MHC-I expression was analyzed by FACS. Bioluminescence imaging measured the different in survival rates in vivo. The overexpression of Erap1 caused a marked upregulation of MHC-I in MP tumors (FIGS. 9J-K), as well as slowed the growth of MP tumors in vivo (FIGS. 9L-M). The overexpression of Tap1 had less of an effect on MHC-I expression and tumor growth. The overexpression of both Erap1 and Tap1 caused a significant delay in the growth of MP tumors in vivo (FIGS. 9L-M). These results suggested that both Erap1 and Tap1 contribute to the resistance of p53-mutant tumors to immune rejection.

### EXAMPLE 8

### TP53 Mutation Regulates ERAP1 Levels in Breast Cancer, Colon Cancer, and Acute Myeloid Leukemia (AML)

This study was directed to assessing the levels of ERAP1 in several tumor samples. If the tumors have significantly lower levels of ERAP1, this would suggest that the tumors would have diminished MHC-I on their surfaces, and have an increased likelihood of being resistant to immunotherapy.

RNA was prepared from a patient's tumor and blood, and both samples were subjected to quantitative RT-PCR using primers specific for ERAP1. Levels of ERAP1 mRNA (as assessed by microarray gene expression analysis from TCGA datasets) were plotted using the cBio web portal (accessible online at http://www.cbioportal.org). Tumors were assigned to the TP53-altered group if they had non-synonymous missense hotspot or truncating (frameshift/ nonsense) mutations. P-values were generated by cBio web portal.

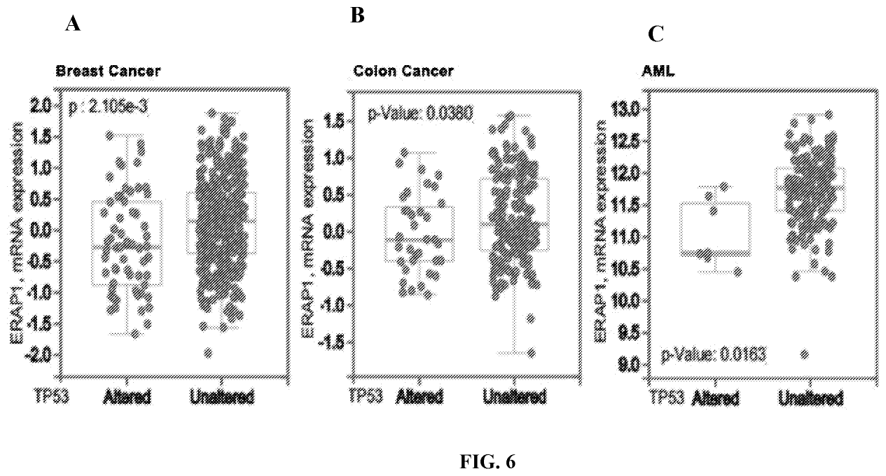

As shown in FIG. 6, TP53-mutant tumors were found to have lower levels of ERAP1 than TP53-wild type tumors in human breast cancer (A), colon cancer (B) and acute myeloid leukemia (C). The results suggested that tumors harboring TP53 mutations had diminished levels of ERAP1, lower expression of MHC-I on their surfaces and therefore a reduced likelihood of responding to immunotherapy.

### EXAMPLE 9

### Restoring the Expression of MHC-I Could Increase the Sensitivity of Cells to Immunotherapy

This study was directed to determining if increasing the expression of MHC-I in cells that lack cell surface localization of MHC-I could increase sensitivity to immunotherapy. The effects of interferon-gamma (IFNγ), tumor necrosis factor alpha (TNF-α), and lymphotoxin beta receptor (LTβ receptor agonist) on MHC-I expression were tested in the tumor models. All cytokines used in vitro were resuspended in DMSO. Cells were treated at 50 ng/ml TNFα, 20 ng/ml of IFNγ, or 1.6 μg/m1 of LTβ receptor agonist. If the expression of MHC-I was restored, this would suggest that the cellular sensitivity to immunotherapy can be restored.

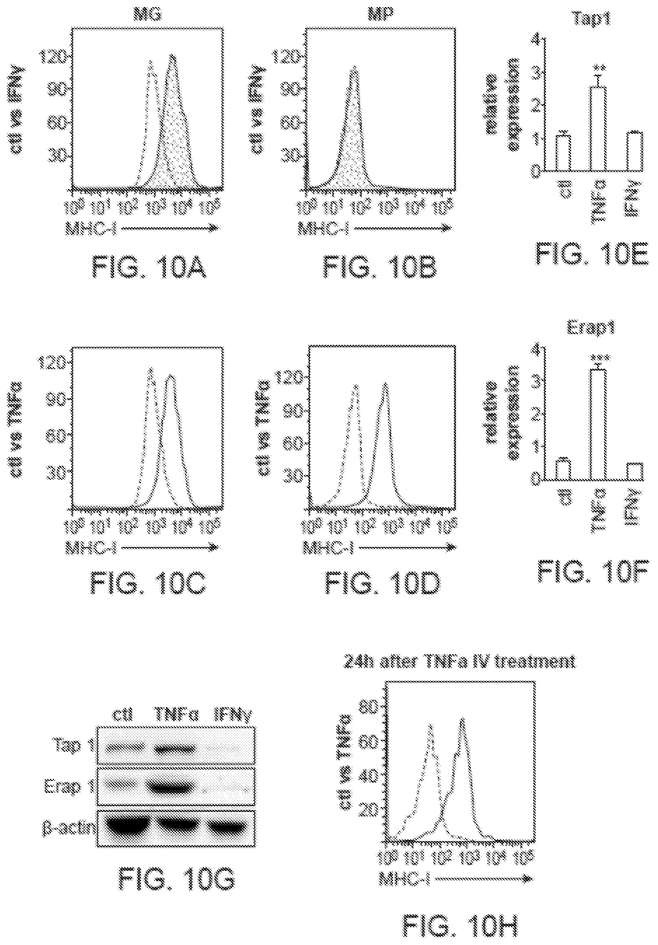

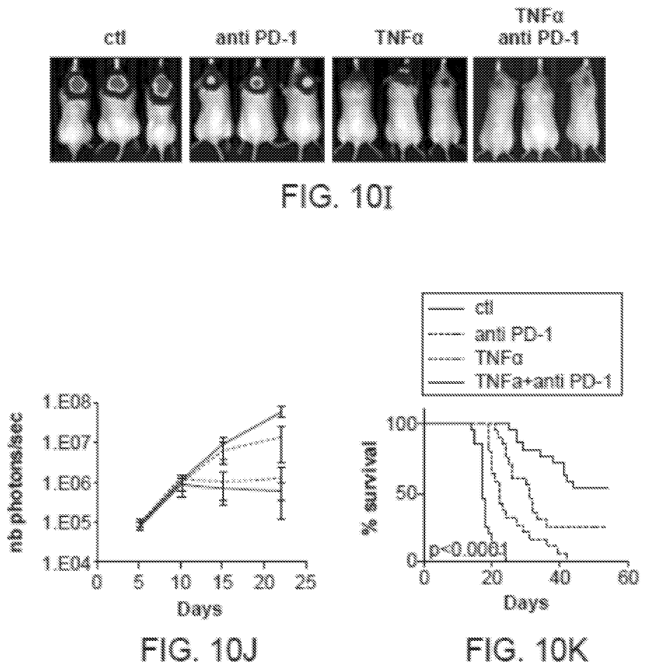

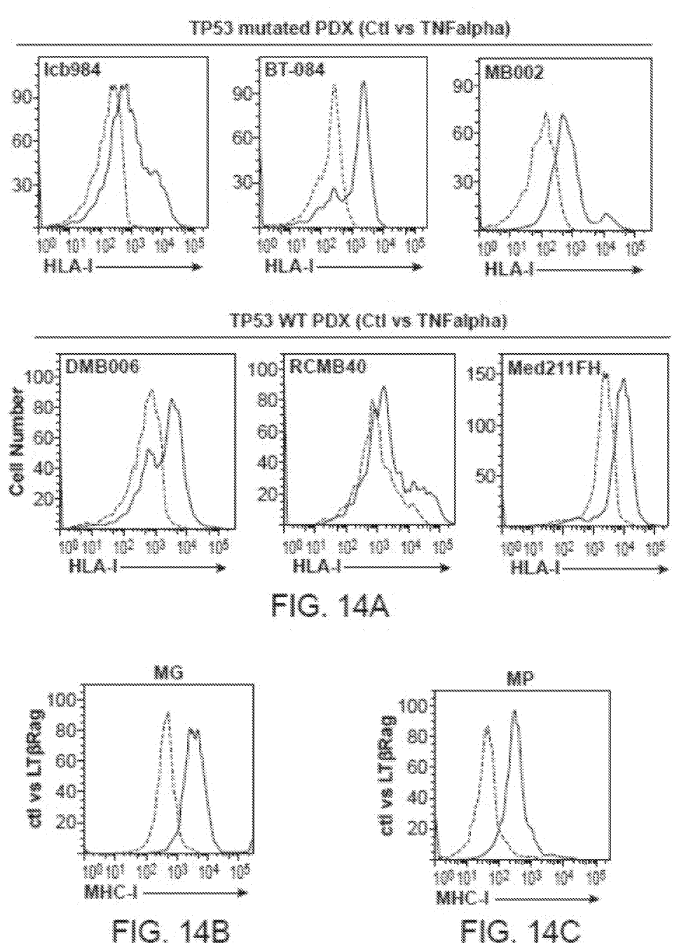

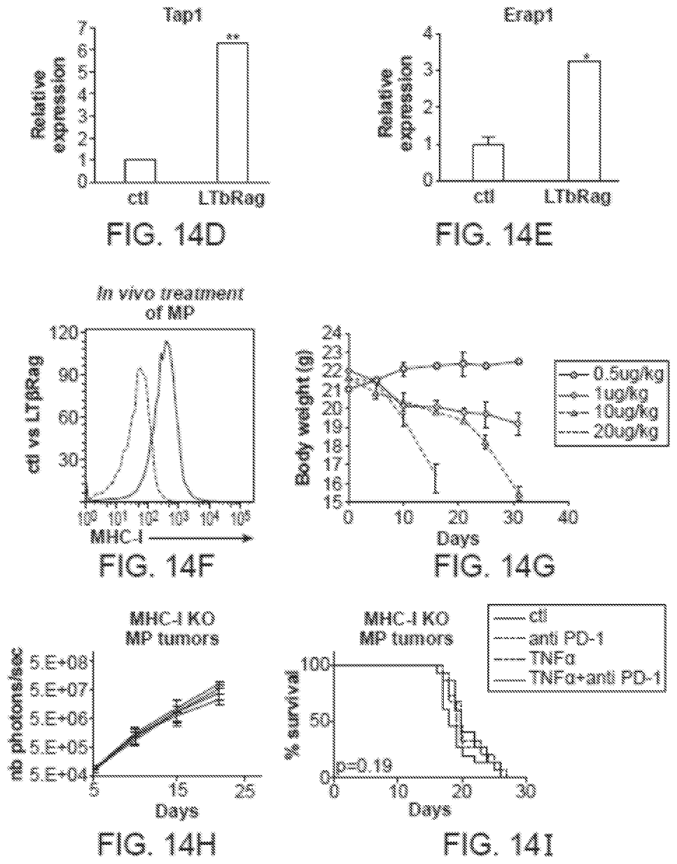

The effects of IFNγ, which has been reported to increase MHC-I expression in a variety of cell types, was tested to determine the effects on MHC-I expression. Although IFNγ caused a significant increase in MHC-I expression in MG tumors, which already express MHC-I (FIG. 10A), it had no effect on MHC-I expression in MP tumors, which lack MHC-I (FIG. 10B). In contrast, TNF-α, which has been reported to enhance MHC-I expression in some cell types, caused a marked increase in MHC-I expression in both MG and MP tumors (FIGS. 10C-D). TNF-α also induced expression of MHC-I in human MB PDXs derived from multiple subgroups (FIG. 14A). Moreover, MHC-I expression was also induced by LTβ receptor agonist, another member of the TNF receptor subfamily (FIGS. 14B-C). TNF-α and LTβ receptor agonist, but not IFNγ, induced expression of Erap1 and Tap1 in MP tumor cells (FIGS. 10E-G, 14D-E). These studies suggest that TNF-α can bypass the effects of loss of p53 by restoring Erap1 and Tap1 expression and permitting surface MHC-I expression in p53 mutant tumor cells.

As shown in in FIG. 10H, intracranial tumor-bearing mice showed a marked upregulation of MHC-I expression 24 hours after treatment with 0.5 μg/kg of TNFα, a dose at which no toxicity is seen, even after daily dosing for several weeks (FIG. 14G). In vivo administration of LTβ receptor agonist also resulted in increased expression of MHCOI in MP tumor cells (FIG. 14F). These studies suggested that low doses of TNF-α or LTβ receptor agonist can be administered safely, can accumulate in brain tumor tissue, and can increase expression of MHC-I in tumor cells

### EXAMPLE 10

### Low Doses of TNF-α can Increase Cell Sensitivity to Immunotherapy

This study was directed to determining if TNF-α could restore sensitivity to T cell-based immunotherapy in p53-mutant medulloblastoma cells. To determine whether doses of TNF-α can be used to sensitize tumor cells to T cell killing, MP tumors were transplanted into aB6 mice and treated with vehicle, with the immune checkpoint inhibitor anti-PD-1, with low-dose TNFα, or with the combination of anti-PD-1 and TNFα. The dosage selected for testing was far below the doses known to cause toxicity (1000 μg/kg or higher). If the tumor cells respond to immunotherapy, this would suggest that TNFαcan increase the expression of MHC-I in tumor cells.

As shown in FIGS. 101-K, mice treated with vehicle have a median survival time of 17 days. Anti-PD-1 alone has little effect on tumor growth or survival (median survival 22 days). TNF-α slows tumor growth and prolongs survival (median survival 31 days), but the combination of anti-PD-1 + TNF-α markedly inhibits tumor growth, leading to a 3.1-fold increase in median survival (median survival 52 days), and to long-term cures in mice (45%). Importantly, these effects are dependent on expression of MHC-I, since no survival benefit is conferred by anti-PD-1 or TNF-α in tumors generated from MHC-I knockout neural stem cells (FIGS. 14H-I). The dose of TNF-α used in mice (0.5 μg/m²) is equivalent to 1.5 μg/m² in humans, which is 130-250-fold lower than the maximum tolerated dose established in Phase I studies of TNF-α (200-400 μg/m²). These results suggest that low doses of TNF-α can be used to increase MHC-I expression and sensitize tumor cells when given alongside immune checkpoint inhibitors.

1.-92. (canceled)

93. A method of treating a patient having a cancer, comprising administering to the patient a low-dose of TNF-a or an LTβ receptor agonist, and an immunotherapy, wherein the low dose of TNF-a comprises a dose that is about 100 fold to about 300 fold lower than a maximum tolerated dose of TNF-a in human.

94. The method of claim 93, wherein the immunotherapy comprises administering to the patient one or more of: an immune checkpoint regulator, an adoptive T-cell therapy, a dendritic cell vaccination, or any combinations thereof.

95. The method of claim 94, wherein the immunotherapy comprises administering to the patient the immune checkpoint regulator, wherein the immune checkpoint regulator comprises an immune checkpoint inhibitor, and wherein the immune checkpoint inhibitor is an antagonist of PD-1, PD-L1, CTLA-4, A2AR, B7-H3, B7-H4, BTLA, IDO, KIR, LAG3, TIM-3, VISTA, CD160, TIGIT or PSGL-1; or an antagonist antibody that binds to PD-1, PD-L1, CTLA-4, A2AR, B7-H3, B7-H4, BTLA, IDO, KIR, LAG3, TIM-3, VISTA, CD160, TIGIT or PSGL-1.

96. The method of claim 94, wherein the immunotherapy comprises administering to the patient the immune checkpoint regulator, wherein the immune checkpoint regulator comprises an immune checkpoint activator, and wherein the immune checkpoint activator is an agonist of costimulation by CD27, CD40, OX40, GITR, CD137, CD28, or ICOS; or an agonist antibody that binds to CD27, CD40, OX40, GITR, CD137, CD28, or ICOS.

97. The method of claim 93, wherein the low dose of TNF-a comprises a dose from 0.6 μg/m² to 40 μg/m².

98. The method of claim 93, wherein the patient has previously been identified as having a reduced likelihood of response to the immunotherapy by a method comprising the steps of:
(i) obtaining a biological sample from said patient and detecting whether the biological sample comprises a loss-of-function TP53 mutation; and
(ii) identifying said patient as having a reduced likelihood of response to the immunotherapy if the biological sample comprises the loss-of-function TP53 mutation.

99. The method of claim 93, wherein the patient has previously been identified as having a reduced likelihood of response to the immunotherapy by a method comprising the steps of:
(i) obtaining a tumor sample from said patient and assaying levels of ERAP1 and TAP1 in said tumor sample; and
(ii) identifying said patient as having a reduced likelihood of response to the immunotherapy if the levels of ERAP1 or TAP1, or both, are lower in the tumor sample than in a reference non-tumor biological sample.

100. The method of claim 99, further comprising assaying a level of MHC-I in the tumor sample and identifying said patient as having a reduced likelihood of response to the immunotherapy if the level of MHC-I is lower in the tumor sample than in the reference non-tumor biological sample.

101. The method of claim 93, wherein the patient has previously been identified as having a reduced likelihood of response to the immunotherapy by a method comprising the steps of:
(i) obtaining a tumor sample from said patient and assaying a level of MHC-I in said tumor sample; and
(ii) identifying said patient as having a reduced likelihood of response to the immunotherapy if the MHC-I level is lower in the tumor sample than in a reference non-tumor biological sample.

102. The method of claim 101, further comprising assaying levels of ERAP1 and TAP1 in the tumor sample and identifying said patient as having a reduced likelihood of response to the immunotherapy if the levels of ERAP1 and TAP1, or both are lower in the tumor sample than in the reference non-tumor biological sample.

103. The method of claim 93, wherein the patient has previously been identified as having a reduced likelihood of response to the immunotherapy by a method comprising the steps of:
obtaining a tumor sample from said patient and performing the following steps:
a) detecting whether the tumor sample comprises a loss-of-function TP53 mutation, and
b) assaying a level of at least one of MHC-I, ERAP1, and TAP1 in said tumor sample; and
(ii) identifying said patient as having a reduced likelihood of response to the immunotherapy if the tumor sample comprises a loss-of-function TP53 mutation or if the level of at least one of MI-IC class 1, ERAP1, and TAP1 in the tumor sample is lower than that in a reference non-tumor biological sample.

104. The method of claim 103, comprising detecting whether the tumor sample comprises the loss-of-function TP53 mutation prior to assaying the level of at least one of MHC-I, ERAP1, and TAP1 in the tumor sample.

105. The method of claim 93, wherein the immunotherapy is administered in combination with a further therapy, wherein the further therapy comprises at least one of: a radiation therapy, a surgery, one or more hormonal agents, or combinations thereof.

106. A method of identifying a cancer patient as having an increased or reduced likelihood of response to an immunotherapy, said method comprising the steps of:
(i) obtaining a tumor sample from said patient and performing the follo g steps:
a) detectin tether the tumor sample comprises a loss-of-function TP53 mutation, and
b) assaying a level of at least one of MHC-I, ERAP1, and TAP1 in said tumor sample; and
(ii) identifying said patient as having an increased likelihood of response to the immunotherapy if the tumor sample does not comprise the loss-of-function TP53 mutation or if the level of at least one of MEW class 1, ERAPI, and TAN in the tumor sample is comparable to that in a reference non-tumor biological sample and identifying said patient has having a reduced likelihood of response to the immunotherapy if the tumor sample comprises a loss-of-function TP53 mutation or if the level of at least one of MHC class 1, ERAP1, and TAP1 in the tumor sample is lower than that in a reference non -tumor biological sample.

107. The method of claim 106, further comprising at least one of (iii) administering the immunotherapy to the patient identified as having the increased likelihood of response in step (ii); or (iv) administering a therapy comprising TNF-α to the patient identified as having the reduced likelihood of response in step (ii)

108. The method of claim 106, wherein the reference non-tumor biological sample is isolated from the same patient.

109. The method of claim 106, herein the cancer comprises a solid tumor, lymphoma, or leukemia.

110. A method for treating a patient having a cancer comprising: (a) selecting for an immunotherapy a patient having a cancer wherein the patient does not comprise a loss-of-function TP53 mutation, and (b) administering to that patient the immunotherapy.

111. The method of claim 109, wherein the immunotherapy comprises administration of one or more of: an immune checkpoint regulator, an adoptive T-cell therapy, a dendritic cell vaccination, or any combinations thereof, and wherein the immune checkpoint regulator comprises an immune checkpoint inhibitor or an immune checkpoint activator.

112. The method of claim 111, wherein the immune checkpoint inhibitor is an antagonist of PD-1, PD-L1, CTLA-4, A2AR, B7-H3, B7-H4, BTLA, IDO, KIR, LAG3, TIM-3, VISTA, CD160, TIGIT or PSGL-1; or an antagonist antibody that binds to PD-1, PD-L1, CTLA-4, A2AR, B7-H3, B7-H4, BTLA, IDO, KIR, LAG3, TIM-3, VISTA, CD160, TIGIT or PSGL-1, and wherein the immune checkpoint activator is an agonist of costimulation by CD27, CD40, OX40, GITR, CD137, CD28, or ICOS; or an agonist antibody that binds to CD27, CD40, OX40, GITR, CD137, CD28, or ICOS.
